(19) United States
(12) Patent Application Publication
(10) Pub. No.: US 2023/0400471 A1
(43) Pub. Date: Dec. 14, 2023

(54) SYSTEMS, COMPOSITIONS AND METHODS OF DETERMINING VIABILITY OF EMBRYOS USING THE SAME

(71) Applicant: BioIncept, LLC, Cherry Hill, NJ (US)

(72) Inventors: Eytan R. Barnea, New York, NY (US); Nicholas Dainiak, Bridgeport, CT (US); Timothy J. Barder, Glen Ellyn, IL (US)

(21) Appl. No.: 18/032,128

(22) PCT Filed: Oct. 14, 2021
(86) PCT No.: PCT/US21/55071

§ 371 Date: Apr. 14, 2023

Related U.S. Application Data
(60) Provisional application No. 63/092,483, filed on Oct. 15, 2020.

(51) Int. Cl. G01N 33/68; C07K 16/18

(52) U.S. Cl. CPC G01N 33/689; C07K 16/18; C07K 2317/34; C07K 2317/565; G01N 2800/367

(57) ABSTRACT

The present disclosure relates generally to compositions and methods of detecting viable embryos for implantation based upon detection and/or quantification of levels or concentrations of the preimplantation factor (PIF) in an embryo culture medium sample.

### CROSS-REFERENCE TO RELATED APPLICATIONS

The present application claims the priority benefit of U.S. Provisional Patent App. Ser. No. 63/092,483, filed Oct. 15, 2020, entitled, “SYSTEMS, COMPOSITIONS AND METHODS OF DETERMINING VIABILITY OF EMBRYOS USING THE SAME”, which is hereby incorporated herein by reference in its entirety.

### TECHNOLOGY FIELD

The present disclosure relates generally to compositions and methods of detecting viable embryos for implantation based upon detection and/or quantification of levels or concentrations of the preimplantation factor (PIF) in an embryo culture medium sample.

### BACKGROUND

Preimplantation factor (PIF), a short peptide of 15 amino acids, present in maternal circulation and is expressed by embryos and placental tissue. PIF was first identified by Barnea and colleagues (Roussev et al., 1996). In the placenta, PIF is expressed in the trophoblastic layer in the first and second trimester while minimally being expressed at term as documented by staining using a specific anti-PIF-antibody (Barnea et al., 2014; Ramu et al., 2013). Synthetic PIF analog (sPIF) replicates native PIF action, modulates peripheral immune cells to achieve tolerance without immune suppression, and has been demonstrated to be effective in autoimmunity models outside pregnancy (Stamatkin et al., 2011). PIF displays essential multi-targeted effects; regulating immunity, promoting embryo-decidual adhesion, and regulating adaptive apoptotic processes in cultured human decidual cells. In addition, PIF promotes trophoblast invasion reflecting an autocrine supporting effect on conceptus development (Barnea, 2004; Mueller et al., 2014).

Earlier studies by Roussev et al., 1996 and other independent investigators (Rosario et al., 2005) confirmed that the presence of PIF in embryo culture media reflects the viability of the embryo. In addition, PIF is also detected in the sera of pregnant women and thus can serve as a biomarker of viable pregnancy. The presence of PIF and its correlation to embryo viability, and successful pregnancy has been well documented. Several test formats including a bio-assay, colorimetric ELISA and chemiluminescent ELISA were used to detect the presence of PIF in embryo culture media and sera (Coulam et al., 1995; Roussev et al., 1995; Statmatkin et al., 2011; Ramu et al., 2013; Rosario et al., 2005).

The bioassay based on autorosette formation between lymphocytes and platelets when treated with pregnant sera diagnoses pregnancy as early as day 2 of mouse pregnancy and on day 2 post-embryo transfer of human in vitro fertilization (IVF) cycles (Roussev et al., 1995; Roussev et al., 1996; Roussev et al., 1996).

The bioassay, however, is based on autorosette formation is cumbersome and requires expert technical personnel.

### SUMMARY OF EMBODIMENTS

Prior attempts in developing diagnostic assays for detecting the preimplantation factor (PIF) in spent embryo culture media have led to little success. This is primarily due to the small amount of PIF, if present, in the spent embryo culture media and the technological challenge of detecting the presence of PIF from low-volume, highly complicated medium, such as embryonic cell culture medium. Thus, sensitivity of the assay is a key. Moreover, prior experiences indicated that not all antibodies to PIF, monoclonal or polyclonal, worked well in detecting PIF in a standard ELISA or microarray format. In the present disclosure, a rabbit monoclonal antibody, specifically the CDR portions thereof, was found to have a high affinity to PIF, which makes it possible to develop an immunoassay with sufficient sensitivity, reliability and reproducibility.

The disclosure relates to a sensitive immunoassay that can be performed with minimal expertise and requires only small quantity of sample for detecting PIF from spent embryo culture media to select viable embryos for transplantation, especially for use with in-vitro fertilization (IVF) clinics and reproductive endocrinologists offices. The disclosure also relates to a monoclonal antibody, or an antigen-binding fragment thereof, that binds to the preimplantation factor (PIF). In some embodiments, the monoclonal antibody of the disclosure comprises a variable region comprising: (a) a complementarity-determining region (CDR) comprising at least about 70% sequence identity to the amino acid sequence of SEQ ID NO: 3, SEQ ID NO: 4 or SEQ ID NO: 5; or (b) a CDR comprising at least about 70% sequence identity to the amino acid sequence of SEQ ID NO: 8, SEQ ID NO: 9 or SEQ ID NO: 10. In some embodiments, the monoclonal antibody of the disclosure comprises a variable region comprising: (a) a complementarity-determining region (CDR) comprising at least about 70% sequence identity to the amino acid sequence of SEQ ID NO: 3, SEQ ID NO: 4 or SEQ ID NO: 5; and (b) a CDR comprising at least about 70% sequence identity to the amino acid sequence of SEQ ID NO: 8, SEQ ID NO: 9 or SEQ ID NO: 10. In some embodiments, the monoclonal antibody of the disclosure comprises a heavy chain variable region comprising at least one complementarity-determining region (CDR) comprising at least about 70% sequence identity to the amino acid sequence of SEQ ID NO: 3, SEQ ID NO: 4 or SEQ ID NO: 5. In some embodiments, the monoclonal antibody of the disclosure comprises a heavy chain variable region comprising at least one complementarity-determining region (CDR) comprising the amino acid sequence of SEQ ID NO: 3, SEQ ID NO: 4 or SEQ ID NO: 5. In some embodiments, the monoclonal antibody of the disclosure further comprises a light chain variable region comprising at least one CDR comprising at least about 70% sequence identity to the amino acid sequence of SEQ ID NO: 8, SEQ ID NO: 9 or SEQ ID NO: 10. In some embodiments, the monoclonal antibody of the disclosure further comprises a light chain variable region comprising at least one CDR comprising the amino acid sequence of SEQ ID NO: 8, SEQ ID NO: 9 or SEQ ID NO: 10.

In some embodiments, the monoclonal antibody of the disclosure comprises (1) a heavy chain variable region comprising a first CDR comprising the amino acid sequence of SEQ ID NO: 3 and a second CDR comprising the amino acid sequence of SEQ ID NO: 4; (2) a heavy chain variable region comprising a first CDR comprising the amino acid sequence of SEQ ID NO: 3 and a second CDR comprising the amino acid sequence of SEQ ID NO: 5; or (3) a heavy chain variable region comprising a first CDR comprising the amino acid sequence of SEQ ID NO: 4 and a second CDR comprising the amino acid sequence of SEQ ID NO: 5. In some embodiments, the monoclonal antibody of the disclosure comprises (1) a light chain variable region comprising a first CDR comprising the amino acid sequence of SEQ ID NO: 8 and a second CDR comprising the amino acid sequence of SEQ ID NO: 9; (2) a light chain variable region comprising a first CDR comprising the amino acid sequence of SEQ ID NO: 8 and a second CDR comprising the amino acid sequence of SEQ ID NO: 10; or (3) a light chain variable region comprising a first CDR comprising the amino acid sequence of SEQ ID NO: 9 and a second CDR comprising the amino acid sequence of SEQ ID NO: 10.

In some embodiments, the monoclonal antibody of the disclosure comprises a heavy chain variable region comprising a first CDR comprising the amino acid sequence of SEQ ID NO: 3, a second CDR comprising the amino acid sequence of SEQ ID NO: 4, and a third CDR comprising the amino acid sequence of SEQ ID NO: 5. In some embodiments, the monoclonal antibody of the disclosure comprises a light chain variable region comprising a first CDR comprising the amino acid sequence of SEQ ID NO: 8, a second CDR comprising the amino acid sequence of SEQ ID NO: 9, and a third CDR comprising the amino acid sequence of SEQ ID NO: 10.

In some embodiments, the monoclonal antibody of the disclosure comprises a heavy chain variable region comprising at least about 70% sequence identity to SEQ ID NO: 2. In some embodiments, the monoclonal antibody of the disclosure comprises a light chain variable region comprising at least about 70% sequence identity to SEQ ID NO: 7. In some embodiments, the monoclonal antibody of the disclosure comprises a heavy chain variable region comprising at least about 90% sequence identity to SEQ ID NO: 2, and a light chain variable region comprising at least about 90% sequence identity to SEQ ID NO: 7. In some embodiments, the monoclonal antibody of the disclosure comprises a heavy chain variable region comprising SEQ ID NO: 2, and a light chain variable region comprising SEQ ID NO: 7. In some embodiments, the monoclonal antibody of the disclosure further comprises a heavy chain constant region. In some embodiments, the monoclonal antibody of the disclosure further comprises a light chain constant region.

In some embodiments, the monoclonal antibody of the disclosure is a rabbit monoclonal antibody. In some embodiments, the monoclonal antibody of the disclosure is capable of binding to a preimplantation factor (PIF), or an immunogenic fragment or epitope thereof. In some embodiments, the monoclonal antibody of the disclosure is specific to the PIF comprising the amino acid sequence of SEQ ID NO: 11. In some embodiments, the monoclonal antibody of the disclosure binds the last 4, 5 or 6 carboxy terminal amino acid residues of SEQ ID NO: 11.

The disclosure further relates to a system comprising: a) any of the disclosed monoclonal antibodies; b) a solid support onto which the one or plurality of monoclonal antibodies are immobilized; and c) at least one substrate. In some embodiments, the system of the disclosure further comprises a fluorimeter or spectrophotometer. In some embodiments, the system of the disclosure further comprises a method of usin the system by performing surface plasmon resonance (SPR) spectroscopy. In some embodiments, the disclosure relates to a method of using the system by performing X-ray crystallography. In some embodiments, the disclosure relates to a method of using the system of the disclosure by performing nuclear magnetic resonance (NMR) spectroscopy. In some embodiments, the disclosure relates to a method of using the system to perform electron microscopy (EM).

In some embodiments, the at least one substrate comprised in the disclosed system is capable of binding to the one or plurality of monoclonal antibodies. In some embodiments, the at least one substrate is fluorogenic, fluorescent, or chemiluminescent fluorescently labeled. In some embodiments, the at least one substrate is a preimplantation factor (PIF), or an immunogenic fragment or epitope thereof. In some embodiments, the PIF comprises at least about 70% sequence identity to the amino acid sequence of SEQ ID NO: 11. In some embodiments, the PIF comprises the amino acid sequence of SEQ ID NO: 11.

In some embodiments, the one or plurality of monoclonal antibodies comprised in any of the disclosed systems are rabbit monoclonal antibodies. In some embodiments, the disclosed system further comprises one or a plurality of mammalian anti-rabbit antibodies. In some embodiments, the one or plurality of monoclonal antibodies are immobilized onto the solid support via the one or plurality of mammalian anti-rabbit antibodies. In some embodiments, the one or plurality of mammalian anti-rabbit antibodies are goat anti-rabbit antibodies.

In some embodiments, the one or more solid supports comprised in any of the disclosed systems are chosen from slides, chips, beads, stripes, plates or membranes.

In some embodiments, the system of the disclosure has a sensitivity of detecting PIF at a concentration from about 0.5 pg/mL to about 500 pg/mL. In some embodiments, the system of the disclosure has a sensitivity of detecting PIF at a concentration from about 1 pg/mL to about 100 pg/mL in a sample. In some embodiments, the system has a sensitivity of detecting PIF at a concentration from about 5 pg/mL to about 50 pg/mL in a sample. In some embodiments, the system has a sensitivity of detecting PIF at a concentration from about 10 pg/mL to about 30 pg/mL in a sample.

Also provided herein are methods of using any of the disclosed monoclonal antibodies or any of the disclosed systems to detect the presence, absence, and/or biologically significant quantity of PIF peptides in a sample. In some embodiments, the disclosure relates to a method of detecting embryo fitness for implantation, the method comprising detecting presence, absence, and/or biologically significant quantity of a PIF peptide, or an immunogenic fragment or epitope thereof, in a sample using any of the disclosed monoclonal antibodies or any of the disclosed systems, wherein the sample is obtained from an embryo culture medium used for culturing one or a plurality of embryos at about Day 3 to about Day 7 after culturing the one or plurality of embryos is fertilized. In some embodiments, the presence of the PIF detected in the sample is indicative of embryo fitness for implantation. In other embodiments, the disclosure relates to a method for detecting a PIF in an embryonic culture medium, the method comprising exposing the embryonic culture medium to any of the disclosed monoclonal antibodies or any of the disclosed systems, wherein the embryo culture medium has been exposed to one or a plurality of embryos from about three to about seven days in culture. In some embodiments, the presence of the PIF in the embryo culture medium is indicative of the presence of one or a plurality of viable embryos in the embryo culture medium. In other embodiments, the disclosure relates to a method of in vitro fertilization of a subject, the method comprising: a) detecting presence, absence, and/or biologically significant quantity of a PIF peptide, or an immunogenic fragment or epitope thereof, in a sample by exposing the sample to any of the disclosed monoclonal antibodies or any of the disclosed systems, wherein the sample is obtained from an embryo culture medium used for culturing one or a plurality of embryos from about 3 to about 7 days, and b) transplanting at least one embryo into the subject, wherein the at least one embryo is obtained from a PIF-positive sample.

In some embodiments, the sample used in any of the disclosed methods is obtained from an embryo culture medium used for culturing one or a plurality of embryos that are from about 3 to about 5 days after fertilization and/or days in culture. In some embodiments, the sample used in any of the disclosed methods is obtained from an embryo culture medium used for culturing one or a plurality of embryos that are about 3 days old. In some embodiments, the disclosed method further comprises exposing the sample to at least one substrate capable of binding to the monoclonal antibody. In some embodiments, the at least one substrate is fluorogenic, fluorescent, or chemiluminescent fluorescently labeled. In some embodiments, the at least one substrate is a preimplantation factor (PIF), or an immunogenic fragment or epitope thereof. In some embodiments, the PIF comprises at least about 70% sequence identity to the amino acid sequence of SEQ ID NO: 11. In some embodiments, the PIF comprises the amino acid sequence of SEQ ID NO: 11. In some embodiments, the monoclonal antibody used in any of the disclosed methods is a rabbit monoclonal antibody.

In some embodiments, the disclosed method further comprises normalizing the quantity of the PIF detected in the sample with a measurement taken from a control sample. In some embodiments, the disclosed method has a sensitivity of detecting PIF at a concentration from about 0.5 pg/mL to about 500 pg/mL in the sample. In some embodiments, the disclosed method has a sensitivity of detecting PIF at a concentration from about 1 pg/mL to about 100 pg/mL in the sample. In some embodiments, the disclosed method has a sensitivity of detecting PIF at a concentration from about 5 pg/mL to about 50 pg/mL in the sample. In some embodiments, the disclosed method has a sensitivity of detecting PIF at a concentration from about 10 pg/mL to about 30 pg/mL in the sample.

In some embodiments, the disclosed method has a sensitivity of detecting PIF at a concentration of at least about 0.5 pg/mL in the sample. In some embodiments, the disclosed method has a sensitivity of detecting PIF at a concentration of at least about 1 pg/mL in the sample. In some embodiments, the disclosed method has a sensitivity of detecting PIF at a concentration of at least about 5 pg/mL in the sample. In some embodiments, the disclosed method has a sensitivity of detecting PIF at a concentration of at least about 10 pg/mL in the sample. In some embodiments, the disclosed method has a sensitivity of detecting PIF at a concentration of at least about 15 pg/mL in the sample. In some embodiments, the disclosed method has a sensitivity of detecting PIF at a concentration of at least about 20 pg/mL in the sample. In some embodiments, the disclosed method has a sensitivity of detecting PIF at a concentration of at least about 25 pg/mL in the sample. In some embodiments, the disclosed method has a sensitivity of detecting PIF at a concentration of at least about 50 pg/mL in the sample. In some embodiments, the disclosed method has a sensitivity of detecting PIF at a concentration of at least about 75 pg/mL in the sample. In some embodiments, the disclosed method has a sensitivity of detecting PIF at a concentration of at least about 100 pg/mL in the sample. In some embodiments, the disclosed method has a sensitivity of detecting PIF at a concentration of at least about 125 pg/mL in the sample. In some embodiments, the disclosed method has a sensitivity of detecting PIF at a concentration of at least about 150 pg/mL in the sample. In some embodiments, the disclosed method has a sensitivity of detecting PIF at a concentration of at least about 200 pg/mL in the sample. In some embodiments, the disclosed method has a sensitivity of detecting PIF at a concentration of at least about 250 pg/mL in the sample. In some embodiments, the disclosed method has a sensitivity of detecting PIF at a concentration of at least about 300 pg/mL in the sample. In some embodiments, the disclosed method has a sensitivity of detecting PIF at a concentration of at least about 350 pg/mL in the sample. In some embodiments, the disclosed method has a sensitivity of detecting PIF at a concentration of at least about 400 pg/mL in the sample. In some embodiments, the disclosed method has a sensitivity of detecting PIF at a concentration of at least about 450 pg/mL in the sample. In some embodiments, the disclosed method has a sensitivity of detecting PIF at a concentration of at least about 500 pg/mL in the sample.

In some embodiments, the disclosed method has a sensitivity to detect at least about 20 ng of PIF in the sample. In some embodiments, the disclosed method has a sensitivity to detect at least about 15 ng of PIF in the sample. In some embodiments, the disclosed method has a sensitivity to detect at least about 10 ng of PIF in the sample. In some embodiments, the disclosed method has a sensitivity to detect at least about 5 ng of PIF in the sample. In some embodiments, the disclosed method has a sensitivity to detect at least about 1 ng of PIF in the sample. In some embodiments, the disclosed method has a sensitivity to detect at least about 500 pg of PIF in the sample. In some embodiments, the disclosed method has a sensitivity to detect at least about 250 pg of PIF in the sample. In some embodiments, the disclosed method has a sensitivity to detect at least about 100 pg of PIF in the sample. In some embodiments, the disclosed method has a sensitivity to detect at least about 50 pg of PIF in the sample. In some embodiments, the disclosed method has a sensitivity to detect at least about 25 pg of PIF in the sample. In some embodiments, the disclosed method has a sensitivity to detect at least about 10 pg of PIF in the sample. In some embodiments, the disclosed method has a sensitivity to detect at least about 1 pg of PIF in the sample. In some embodiments, the disclosed method has a sensitivity to detect at least about 0.5 pg of PIF in the sample. In some embodiments, the disclosed method has a sensitivity to detect at least about 0.1 pg of PIF in the sample. In some embodiments, the disclosed method has a sensitivity to detect at least about 0.05 pg of PIF in the sample. In some embodiments, the disclosed method has a sensitivity to detect at least about 0.01 pg of PIF in the sample.

In some embodiments, the disclosed method has a sensitivity to detect from about 0.01 pg to about 20 ng of PIF in the sample. In some embodiments, the disclosed method has a sensitivity to detect from about 0.05 pg to about 15 ng of PIF in the sample. In some embodiments, the disclosed method has a sensitivity to detect from about 0.1 pg to about 10 ng of PIF in the sample. In some embodiments, the disclosed method has a sensitivity to detect from about 0.5 pg to about 5 ng of PIF in the sample. In some embodiments, the disclosed method has a sensitivity to detect from about 1 μg to about 1 ng of PIF in the sample. In some embodiments, the disclosed method has a sensitivity to detect from about 1 μg to about 500 pg of PIF in the sample. In some embodiments, the disclosed method has a sensitivity to detect from about 0.01 pg to about 100 pg of PIF in the sample. In some embodiments, the disclosed method has a sensitivity to detect from about 1 μg to about 50 pg of PIF in the sample.

The disclosure additionally relates to a method of detecting a protein that binds to a PIF peptide, the method comprising exposing the PIF, or an immunogenic fragment or epitope thereof, with any of the disclosed monoclonal antibodies in the presence of a test protein under conditions wherein, in the absence of the test protein, a pre-determined quantity of the PIF would bind the monoclonal antibody, and determining if the quantity of the PIF bound to the monoclonal antibody is decreased in the presence of the test protein, the decrease indicating that the test protein binds to the PIF. In some embodiments, the PIF peptide used in such methods is fluorogenic, fluorescent, or chemiluminescent fluorescently labeled. In some embodiments, the PIF peptide comprises at least about 70% sequence identity to SEQ ID NO: 11. In some embodiments, the PIF peptide comprises the amino acid sequence of SEQ ID NO: 11. In some embodiments, the monoclonal antibody used in such methods is a rabbit monoclonal antibody.

The disclosure further relates to a nucleic acid molecule encoding any of the disclosed monoclonal antibodies. In some embodiments, the disclosed nucleic acid molecule comprises at least about 70% sequence identity to SEQ ID NO: 1. In some embodiments, the disclosed nucleic acid molecule comprises at least about 70% sequence identity to SEQ ID NO: 6. In some embodiments, the disclosed nucleic acid molecule comprises SEQ ID NO: 1. In some embodiments, the disclosed nucleic acid molecule comprises SEQ ID NO: 6. In some embodiments, the disclosed nucleic acid molecule comprises SEQ ID NO: 1 and SEQ ID NO: 6.

The disclosure further relates to a nucleic acid molecule encoding any of the disclosed monoclonal antibodies or a monoclonal antibody comprising one or a plurality of amino acids that comprise at least 90% sequence identity to one a plurality of disclosed CDR sequences. The disclosure also relates to a vector comprising one or plurality of nucleic acid molecules encoding any of the disclosed monoclonal antibody embodiments. In some embodiments, the disclosed vector comprises any of the above disclosed nucleic acid molecules. In some embodiments, the vector is a plasmid.

The disclosure further relates to a cell comprising a nucleic acid molecule encoding any of the disclosed monoclonal antibody embodiments. In some embodiments, the cell comprises any of the above disclosed nucleic acid molecules. In some embodiments, the cell comprises any of the above disclosed vectors. In some embodiments, the cell actively expresses the monoclonal antibody. In some embodiments, the cell is actively secreting the monoclonal antibody. In some embodiments, the cell is a hybridoma. The disclosure relates to a method of manufacturing any of the disclosed monoclonal antibody embodiment comprising synthesizing the amino acid residues in solution or solid phase. The disclosure also relates to a method of manufacturing any of the disclosed monoclonal antibody embodiment comprising synthesizing the amino acid residues in solution or solid phase.

### BRIEF DESCRIPTION OF THE DRAWINGS

FIG. 1 depicts a schematic of the surface plasmon resonance (SPR) assay described in Example 3 (FIP: scrambled PIF).

FIG. 2A-2D depict the binding specificity of the rabbit monoclonal antibody Clone #30 to PIF by SPR. FIG. 2A (SCR assay 1) and FIG. 2B (SCR assay 2) show no binding between scrambled PIF and Clone #30. FIG. 2C (PIF assay 1) and FIG. 2D (PIF assay 2) show a significant and concentration dependent binding between PIF and Clone #30.

FIG. 3 depicts a representative biochip layout for standard curve and B₀ value for duplicate analysis of 2 embryo culture media (ECM) samples as described in Example 6.

FIG. 4A and FIG. 4B depict the standard curves obtained from the Log based curve fit calculation (FIG. 4A) and 4PL based curve fit calculation (FIG. 4B) described in Example 6.

FIG. 5 depicts a bar chart showing the calculated levels of nPIF-1₁₅ in each ECM sample analyzed in Example 6 based on both Log based curve fit calculation (gray bars) and 4PL based curve fit calculation (dark gray bars). The average level of nPIF-1₁₅ in each ECM sample is shown in light gray bar.

FIG. 6 depicts a bar chart showing the calculated levels of nPIF-1₁₅ in each of the 20 3-day single embryo cultured ECM samples analyzed in the first part of Example 7 based on both Log based curve fit calculation (gray bars) and 4PL based curve fit calculation (dark gray bars). The average level of nPIF-1₁₅ in each ECM sample is shown in light gray bar.

FIG. 7 depicts a bar chart showing the calculated levels of nPIF-1₁₅ in each of the 50 3-day single embryo cultured ECM samples analyzed in the second part of Example 7 based on both Log based curve fit calculation (light gray bars) and 4PL based curve fit calculation (dark gray bars). The average level of nPIF-1₁₅ in each ECM sample is shown in gray bar (middle bar).

FIG. 8 depicts a histogram for the 50 3-day single embryo cultured ECM samples analyzed in the second part of Example 7 (5:1 diluted).

FIG. 9 depicts the standard curves obtained from the ELISA assay described in Example 8.

### DETAILED DESCRIPTION OF EMBODIMENTS

Before the present compositions and methods are described, it is to be understood that this disclosure is not limited to the particular molecules, compositions, methodologies or protocols described, as these may vary. It is also to be understood that the terminology used in the description is for the purpose of describing the particular versions or embodiments only, and is not intended to limit the scope of the present disclosure which will be limited only by the appended claims. It is understood that these embodiments are not limited to the particular methodology, protocols, cell lines, vectors, and reagents described, as these may vary. It also is to be understood that the terminology used herein is for the purpose of describing particular embodiments only, and is not intended to limit the scope of the present embodiments or claims.

Although any methods and materials similar or equivalent to those described herein can be used in the practice or testing of embodiments of the present disclosure, the preferred methods, kits, and materials are now described. All publications mentioned herein are incorporated by reference. Nothing herein is to be construed as an admission that the disclosure is not entitled to antedate such disclosure by virtue of prior disclosure.

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

The term “at least” prior to a number or series of numbers (e.g. “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

Ranges provided herein are understood to include all individual integer values and all subranges within the ranges.

The term “addressable location” as used herein means a discrete surface area or position on a solid support onto which one or a plurality of substrates, such as monoclonal antibody disclosed herein, are immobilized such that exposure of the one or plurality of substrates to a sample for a sufficient time period results in contact between the substrate (e.g., antibody) and the target to which the substrate are to detect (e.g., peptide or protein). In some embodiments, the disclosure relates to an array comprising one or a plurality of addressable locations of the array with a volume capacity of from about 1 μL to about 10 μL. In some embodiments, the disclosure relates to an array comprising one or a plurality of addressable locations of the array with a volume capacity of about 10 μL. In some embodiments, the disclosure relates to an array comprising one or a plurality of addressable locations of the array with a volume capacity of about 20 μL. In some embodiments, the disclosure relates to an array comprising one or a plurality of addressable locations of the array with a volume capacity of about 30 μL. In some embodiments, the disclosure relates to an array comprising one or a plurality of addressable locations of the array with a volume capacity of about 40 μL. In some embodiments, the disclosure relates to an array comprising one or a plurality of addressable locations of the array with a volume capacity of about 50 μL. In some embodiments, the disclosure relates to an array comprising one or a plurality of addressable locations of the array with a volume capacity of about 60 μL. In some embodiments, the disclosure relates to an array comprising one or a plurality of addressable locations of the array with a volume capacity of about 70 μL. In some embodiments, the disclosure relates to an array comprising one or a plurality of addressable locations of the array with a volume capacity of about 80 μL. In some embodiments, the disclosure relates to an array comprising one or a plurality of addressable locations of the array with a volume capacity of about 90 μL. In some embodiments, the disclosure relates to an array comprising one or a plurality of addressable locations of the array with a volume capacity of about 100 μL. In some embodiments, the one or a plurality of addressable locations of the array have a volume capacity of no more than about 250 μL. In some embodiments, the one or a plurality of addressable locations of the array have a volume capacity of no more than about 200 μL. In some embodiments, the disclosure relates to an array comprising one or a plurality of addressable locations of the array with a volume capacity of no more than about 150 μL. In some embodiments, the one or a plurality of addressable locations of the array have a volume capacity of no more than about 100 μL. In some embodiments, the one or a plurality of addressable locations of the array have a volume capacity of no more than about 90 μL. In some embodiments, the one or a plurality of addressable locations of the array have a volume capacity of no more than about 80 μL In some embodiments, the one or a plurality of addressable locations of the array have a volume capacity of no more than about 70 μL. In some embodiments, the one or a plurality of addressable locations of the array have a volume capacity of no more than about 60 μL. In some embodiments, the one or a plurality of addressable locations of the array have a volume capacity of no more than about 50 μL. In some embodiments, the one or a plurality of addressable locations of the array have a volume capacity of no more than about 40 μL. In some embodiments, the one or a plurality of addressable locations of the array have a volume capacity of no more than about μL. In some embodiments, the one or a plurality of addressable locations of the array have a volume capacity of no more than about 20 μL. In some embodiments, the one or a plurality of addressable locations of the array have a volume capacity of no more than about 10 μL. In some embodiments, the one or a plurality of addressable locations of the array have a volume capacity from about 10 μL to about 100 μL. In some embodiments, the one or plurality of addressable locations of the array is spotted manually by a pipet or automatically by a robotic device.

The term “antibody,” as used herein, broadly refers to any immunoglobulin (Ig) molecule comprised of four polypeptide chains, two heavy (H) chains and two light (L) chains, or any functional fragment, mutant, variant, or derivative thereof, which retains the essential epitope binding features of an Ig molecule. Such mutant, variant, or derivative antibody formats are known in the art. Non-limiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

As used herein, the term “CDR” refers to the complementarity determining region within antibody variable sequences. In some embodiments, there are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia et al., J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H1}; (ii) V_{H}-C_{H2}; (iii) VH-CH3; (iv) V_{H}-C_{H1}-C_{H2}; (V) V_{H}-C_{H1}-C_{H2}-C_{H3}; (vi) V_{H}-C_{H2}-C_{H3}; (vii) V_{H}-C_{L}; V_{L}-C_{H1}; (ix) V_{L}-C_{H2}; (X) V_{L}-C_{H3}; (xi) V_{L}-C_{H1}-C_{H2}; (Xii) V_{L}-C_{H1}-C_{H2}-C_{H3}; V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least about 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As used herein, the term “fragment” is defined as a physically contiguous portion of the primary structure of a biomolecule. In the case of polypeptides, a fragment may be defined by a contiguous portion of the amino acid sequence of a protein and may be at least about 3-5 amino acids, at least about 6-10 amino acids, at least about 11-15 amino acids, at least about 16-24 amino acids, at least about 25-30 amino acids, at least about 30-45 amino acids and up to the full length of the protein minus a few amino acids. In the case of polynucleotides, a fragment is defined by a contiguous portion of the nucleic acid sequence of a polynucleotide and may be at least about 9-nucleotides, at least about 15-30 nucleotides, at least about 31-45 nucleotides, at least about 46-74 nucleotides, at least about 75-90 nucleotides, and at least about 90-130 nucleotides. In some embodiments, fragments of biomolecules are immunogenic fragments.

In some embodiments, the term “functional fragment” means any portion of a polypeptide or amino acid sequence that is of a sufficient length to retain at least partial biological function that is similar to or substantially similar to the wild-type polypeptide or amino acid sequence upon which the fragment is based. A functional fragment is a fragment of the antibodies disclosed herein and maintains at least a partial binding affinity to its target. In some embodiments, a functional fragment has a length of at least about 10, about 20, about 30, about 40, about 50, about about 70, about 80, about 90, or about 100 contiguous amino acids. In some embodiments, the functional fragment is a fragment of the antibodies disclosed herein and has a length of at least about 25, about 50, about 75, about 100, about 150, about 200, about 250, about 300, about 350, about 400, about 450, or about 500 amino acids.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

The “variable domain” (variable domain of a light chain (VL), variable domain of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a beta-sheet conformation and the CDRs may form loops connecting the beta-sheet structure. The CDRs in each chain are held in their three-dimensional structure by the framework regions and form together with the CDRs from the other chain an antigen binding site. References to “VH” refer to the variable domain of an immunoglobulin heavy chain, including that of an antibody fragment, such as Fv, scFv, dsFv or Fab. References to “VL” refer to the variable domain of an immunoglobulin light chain, including that of an Fv, scFv, dsFv or Fab.

The term “antigen binding portion” or “antigen binding fragment” of an antibody (or simply “antibody portion” or “antibody fragment”), as used herein, refers to one or more fragments of an antibody that retain the ability to specifically bind to an antigen (e.g., hCD40). It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” or “antigen binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen binding fragment” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

Full length antibodies comprise immunoglobulin constant regions of one or more immunoglobulin classes. Immunoglobulin classes include IgG, IgM, IgA, IgD, and IgE isotypes and, in the case of IgG and IgA, their subtypes. In a preferred embodiment, a full length antibody of the disclosure has a constant domain structure of an IgG type antibody.

The terms “Kabat numbering,” “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen-binding portion thereof (Kab at et al. (1971) Ann. NY Acad, Sci. 190:382-391; Kab at, E.A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 44 to 51 for CDR1, amino acid positions 69 to 75 for CDR2, and amino acid positions 112 to 120 for CDR3. For the light chain variable region, the hypervariable region ranges from amino acid positions 49 to 55 for CDR1, amino acid positions 73 to 75 for CDR2, and amino acid positions 112 to 121 for CDR3.

The term “multispecific antibody” refers to an antibody or antibody-like molecule, or fragment thereof, capable of binding two or more related or unrelated targets, or antigens. Antibody specificity refers to selective recognition of the antibody for a particular epitope, or amino acid sequence, of an antigen. Natural antibodies, for example, are monospecific. Bispecific antibodies are antibodies which have two different antigen-binding specificities. Trispecific antibodies accordingly are antibodies of the disclosure which have three different antigen-binding specificities. Tetraspecific antibodies according to the disclosure are antibodies which have four different antigen-binding specificities.

The term “immunogenic fragment” or “epitope” includes any polypeptide determinant capable of specific binding to an antibody. In certain embodiments, immunogenic fragment or epitope determinant include chemically active surface groupings of molecules such as amino acids, sugar side chains, phosphoryl, or sulfonyl, and, in certain embodiments, may have specific three dimensional structural characteristics, and or specific charge characteristics. An immunogenic fragment or epitope is a region of an antigen that is bound by an antibody.

The term “antigen” refers to a polypeptide that can stimulate the production of antibodies or a T cell response in an animal, including polypeptides that are injected or absorbed into an animal. An antigen reacts with the products of specific humoral or cellular immunity.

The term “human antibody,” as used herein, is intended to include non-naturally occurring human antibodies. The term includes antibodies that are recombinantly produced in a non-human mammal, or in cells of a non-human mammal. The term is not intended to include antibodies isolated from or generated in a human subject.

The antibodies of the disclosure can, in some embodiments, be recombinant antibodies. The term “recombinant antibody,” as used herein, is intended to include all antibodies that are prepared, expressed, created or isolated by recombinant means, such as antibodies expressed using a recombinant expression vector transfected into a host cell (described further below), antibodies isolated from a recombinant, combinatorial antibody library (described further below), antibodies isolated from an animal (e.g., a mouse) that is transgenic for immunoglobulin (e.g. human) genes (see e.g., Taylor et al. (1992) Nucl. Acids Res. 20:6287-6295) or antibodies prepared, expressed, created or isolated by any other means that involves splicing of one species of immunoglobulin gene sequences to other DNA sequences. In certain embodiments, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while related to human germline VH and VL sequences, may not naturally exist within the human antibody germline repertoire in vivo. Recombinant antibodies can be from any mammal, such as, but not limited to, human, rat, mouse, rabbit, dog, horse, pig, etc.

The antibodies of the disclosure can be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody can be substantially free of other cellular material and/or chemicals.

As used herein, the term “subject,” “individual” or “patient,” used interchangeably, means any animal, including mammals, such as mice, rats, other rodents, rabbits, dogs, cats, swine, cattle, sheep, horses, or primates, such as humans. In some embodiments, the subject is a human seeking to become pregnant. In some embodiments, the subject is a human being treated for in vitro fertilization. In some embodiments, the subject is a human undergoing treatment for infertility.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

As used herein, the term “mammal” means any animal in the class Mammalia such as rodent (i.e., mouse, rat, or guinea pig), monkey, cat, dog, cow, horse, pig, or human. In some embodiments, the mammal is a human. In some embodiments, the mammal refers to any non-human mammal. The present disclosure relates to any of the methods or compositions of matter wherein the sample is taken from a mammal or non-human mammal. The present disclosure relates to any of the methods or compositions of matter wherein the sample is embryonic culture medium exposed to embryonic cells taken from a human or non-human primate.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

As used herein, “cell culture” means growth, maintenance, transfection, or propagation of cells, tissues, or their products. As used herein, “culture medium” refers to any solution capable of sustaining the growth of the targeted cells either in vitro or in vivo, or any solution with which targeted cells or exogenous nucleic acids are mixed before being applied to cells in vitro or to a patient in vivo. In some embodiments, culture medium means solution capable of sustaining the growth of the targeted cells either in vitro. In some embodiments, the “cell culture” is an “embryo culture” for growth, maintenance, or propagation of embryos in vitro for transplantation. As used herein, “spent culture medium” refers to a culture medium that has been used for growth, maintenance, or propagation of targeted cells or embryos. In some embodiments, spent culture medium is a spent embryo culture medium that has been used for growth, maintenance, or propagation of one or a plurality of embryos.

As used herein, the term “sample” refers generally to a limited quantity of something which is intended to be similar to and represent a larger amount of that thing. In some embodiments, a sample of the present disclosure is an embryo culture medium. In some embodiments, the sample is a spent embryo culture medium taken from a single embryo culture or a culture of a plurality of embryos. In some embodiments, a sample to be tested is compared to a “control sample” that is known to contain or not to contain PIF. As used herein, “control sample” or “reference sample” refer to samples with a known presence, absence, or pre-determined quantity of substance being measured, that is used for comparison against an experimental or testing sample.

As used herein, the terms “biologically significant” refers to an amount or concentration of PIF in a sample whose quantity of binding that is detected and is statistically significant as compared to a control when the amount or concentration is normalized against the quantity of detected binding in the control. In some embodiments, the terms is used to describe the amount of PIF that is present in a sample at a level sufficient to indicate the presence of at least one viable embryo in the embryo culture media from which the sample is obtained. In some embodiments, the biologically significant amount of PIF is the amount sufficient to characterize an embryo culture media from which the sample is obtained as comprising at least one embryo that is fit for implantation. In some embodiments, the biologically significant amount of PIF is the amount sufficient to characterize an embryo culture media from which the sample is obtained as comprising at least one viable embryo. In some embodiments, the terms is used to describe the amount of PIF that is present in a sample at a level sufficient to indicate with a degree of certainty from about 50% to about 75% that the at least one viable embryo exposed to the embryo culture media from which the sample is obtained is suitable for implantation.

As used herein, the phrase “in need thereof” means that the animal or mammal has been identified or suspected as having a need for the particular method or treatment. In some embodiments, the identification can be by any means of diagnosis or observation. In any of the methods and treatments described herein, the animal or mammal can be in need thereof. In some embodiments, the subject in need thereof is a human seeking to become pregnant. In some embodiments, the subject in need thereof is a human being treated for in vitro fertilization. In some embodiments, the subject in need thereof is a human undergoing treatment for infertility.

As used herein, the term “substrate” refers to any molecule that may bind or associate, directly or indirectly, covalently or non-covalently, to any of the target peptides and whose association or binding is detectable using the methods disclosed herein. In some embodiments, the substrate of the disclosure is capable of binding to the one or plurality of the monoclonal antibodies disclosed herein. In some embodiments, the substrate of the disclosure is fluorogenic, fluorescent, or chemiluminescent fluorescently labeled. In some embodiments, the substrate of the disclosure is a preimplantation factor (PIF), or an immunogenic fragment or epitope thereof. In some embodiments, the substrate of the disclosure is a preimplantation factor (PIF), or an immunogenic fragment or epitope thereof comprising at least about 70%, at least about 75%, at least about 80%, at least about 85%, at least about 86%, at least about 90%, at least about 93%, at least about 95%, at least about 96%, at least about 97%, at least about 98%, or at least about 99% sequence identity to the amino acid sequence of SEQ ID NO: 11. In some embodiments, the substrate of the disclosure is a preimplantation factor (PIF), or an immunogenic fragment or epitope thereof comprising the amino acid sequence of SEQ ID NO: 11.

As used herein, the terms “fluorogenic” and “fluorescent” refer to any molecule (dye, quantum dot, peptide, or fluorescent marker) that emits a known and/or detectable wavelength of light upon exposure to a known wavelength of light. In some embodiments, the substrates of the disclosure are covalently or non-covalently attached to a fluorogenic molecule. In some embodiments, the attachment of the fluorogenic molecule to the substrate of the disclosure creates a chimeric molecule capable of a fluorescent emission or emissions upon exposure to the known wavelength of light, such that the substrate of the disclosure associated with the monoclonal antibody of the disclosure is quantifiable in the presence of a fluorimeter. In some embodiments, the fluorogenic molecule is a coumarin-based dye or rhodamine-based dye with fluorescent emission spectra measureable or quantifiable in the presence of or exposure to a predetermined wavelength of light. In some embodiments, the fluorogenic molecule comprises rhodamine. In some embodiments, the fluorogenic molecule comprises rhodamine-100. Coumarin-based fluorogenic molecules are known in the art, for example in U.S. Pat. Nos. 7,625,758 and 7,863,048, which are herein incorporated by reference in their entireties. In some embodiments, the fluorogenic molecules are a component to, covalently bound to, non-covalently bound to, intercalated with any of the substrates disclosed herein. In some embodiments, the fluorogenic molecules are chosen from 7-amino-4-carbamoylmethylcoumarin (ACC) or 7-amino-4-methylcoumarin (AMC). In some embodiments, the fluorogenic molecule is a fluorescein molecule. In some embodiments, the fluorogenic molecule is capable of emitting a resonance wave detectable and/or quantifiable by a fluorimeter. “Fluorescence microscopy,” which uses the fluorescence to generate an image, may be used to detect the presence, absence, or quantity of a fluorescent molecule. In some embodiments, fluorescence microscopy comprises measuring fluorescence resonance energy transfer (FRET) within a FRET-based assay.

A “chemiluminescent fluorescently labeled” substrate refers to any molecule (dye, peptide, or chemiluminescent marker) that emits a known and/or detectable wavelength of light as the result of a chemical reaction. Chemiluminescence differs from fluorescence or phosphorescence in that the electronic excited state is the product of a chemical reaction rather than of the absorption of a photon. Non-limiting examples of chemiluminescent probes are luciferin and aequorin molecules. In some embodiments, a chemiluminescent molecule is covalently or non-covalently attached to the substrate of the disclosure, such that the excited electronic state can be quantified to determine the presence, absence and/or quantity of the substrate in a test.

“Limit of detection,” as that term is used herein, includes the lowest concentration at which one can identify a sample as containing a molecule of interest, e.g., the first non-zero value. It can be defined by the variability of zeros and the slope of the standard curve. For example, the limit of detection of an assay may be determined by running a standard curve, determining the standard curve zero value, and adding 2 standard deviations to that value. A concentration of the molecule of interest that produces a signal equal to this value is the “lower limit of detection” concentration.

A “score” is a numerical value that may be assigned or generated after normalization of the value based upon the presence, absence, or quantity of PIF peptides in the sample. In some embodiments, the score is normalized in respect to a control raw data value.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may he performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about less than about 0.01, and less than about 0.001.

As used herein, “specific to,” “specific for” or “specifically binds to” means that the binding affinity of a substrate to a specified target molecule, such as the binding affinity of a PIF peptide to an anti-PIF antibody, is statistically higher than the binding affinity of the same substrate to a generally comparable, but non-target molecule. The substrate's K_{D} to each molecule can be compared to assess the binding specificity of the substrate to a particular target molecule. In some embodiments, the term “specific to,” “specific for” or “specifically binds to” refers to the ability of an antibody to bind to PIF with greater affinity than it binds to an unrelated control protein. In some embodiments, the antibodies of the disclosure bind to PIF with an affinity that is at least about 50, about 100, about 250, about 500, about 1000, or about 10,000 times greater than the affinity for a control protein. In some embodiments, the antibodies of the disclosure have a binding affinity K_{D} for PIF of less than or equal to about 1×10⁻⁷ M, less than or equal to about 1×10⁻⁸ M, less than or equal to about 1×10⁻⁹ M, less than or equal to about 1×10⁻¹⁰ M, less than or equal to about 1×10⁻¹¹ M, or less than or equal to about 1×10⁻¹² M.

As used herein, the term “variant” or “variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule or amino acid sequence of the disclosure will have at least about 70%, 75%, 80%, 85%, 87%, 90%, 91%, 92%, 93%, 94%, 95%, 96%, 97%, 98%, 99% or more sequence identity to that particular polynucleotide as determined by sequence alignment programs and parameters as described elsewhere herein.

Variants of a particular nucleic acid molecule of the disclosure can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 87%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 87%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as about 1-15 amino acid residues, as few as about 1-10, such as about 6-10, as few as about 20, about 15, about 10, about 9, about 8, about 7, about 6, about 5, as few as about 4, about 3, about 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), comprising additional, exogenous DNA, RNA or hybrid DNA or RNA molecules that can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide. The disclosure relates to any one or plurality of vectors that comprise nucleic acid sequences encoding any one or plurality of the amino acid sequences disclosed herein.

Anti-PIF Antibodies

The present disclosure relates to anti-PIF monoclonal antibodies that bind to PIF peptides, or immunogenic fragments or epitopes thereof. The term “monoclonal antibody” or “mAb” refers to a homogeneous antibody population capable of binding to the same antigen(s), for example, to the same epitope within the antigen. This term is not intended to be limited with regard to the source of the antibody or the manner in which it is made.

A non-limiting example of the monoclonal antibodies of the disclosure may comprise a heavy chain variable region (HCVR) having the following sequences, or functional fragments or variants thereof.

Heavy chain variable region (HCVR) nucleic acid sequence:
(SEQ ID NO: 1)
atggagactgggctgcgctggcttctcctggtcgctgtgctcaaaggtgtccagtgtcggtcgctggaggagtccgg
gggtcgcctggtcacgcctgggacacccctgacactcacctgcacagtctctggaatcgacctcagcacctattcaat
gagttgggtccgccaggctccaggggagggcctggaatggatcgccaccattaatactggtggtagcgcatactac
gcgaactgggcgaaaggccgattcaccatctccaaaacctcgaccacggtgactctgaaaatgaccagtctgacaa
ccgaggacacggccacctatttctgtgccagaacgattagtgctgataatttgtggggccacggcaccctggtcacc
gtctcctcagggcaacctaaggctccatcagtcttcccactggccccctgctgcggggacacacccagctccacggt
caccctgggttgtcttgtgaagggatacctcccggaacccgtgaccgtgacctggaactccggcaccctgaccaatg
gagtgcggaccttcccgagcgtcaggcagtcctccgggttgtacagcttgtctagcgtggtgtccgtgacgtcgtcaa
gccagcctgtgacttgcaatgtggcccatccggccaccaacaccaaggtcgacaagaccgtggcgccttccacctg
ttccaagcccacttgcccgccgcctgagctcctgggaggaccgtccgtgttcatcttccctccaaaacccaaggatac
cctgatgattagccgcactcccgaagtcacttgcgtggtcgtggacgtgtcgcag
HCVR amino acid sequence:
(SEQ ID NO: 2)
METGLRWLLLVAVLKGVQCRSLEESGGRLVTPGTPLTLTCTVSGIDLSTY
SMSWVRQAPGEGLEWIATINTGGSAYYANWAKGRFTISKTSTTVTLKMT
SLTTEDTATYFCARTISADNLWGHGTLVTVSSGQPKAPSVFPLAPCCGDT
PSSTVTLGCLVKGYLPEPVTVTWNSGTLTNGVRTFPSVRQSSGLYSLSSV
VSVTSSSQPVTCNVAHPATNTKVDKTVAPSTCSKPTCPPPELLGGPSVFIFP
PKPKDTLMISRTPEVTCVVVDVSQ

Based on the IMGT® (the international ImMunoGeneTics information System®) software (http://www.imgt.org) prediction, this HCVR has the following complementarity-determining regions (CDRs). In some embodiments, the monoclonal antibody orantibody fragment thereof comprises 1, 2, 3, 4, 5, 6 or more CDR sequences chosen from: SEQ ID NO:3, SEQ ID NO:4, SEQ ID NO:5, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, and SEQ ID NO:32.
HCVR CDR1: GIDLSTYS (SEQ ID NO: 3)
HCVR CDR2: INTGGSA (SEQ ID NO: 4)
HCVR CDR3: ARTISADNL (SEQ ID NO: 5)

This HCVR may alternatively comprise the following CDRs:

| | |
|---|---|
| Sequence | SEQ ID NO: |

HCVR CDR1

| | |
|---|---|
| SGIDLSTYSN | 12 |
| VSGIDLST | 13 |
| IDLSTYSM | 14 |
| STYSMSW | 15 |
| VSGIDLSTYSMS | 16 |
| IDLSTYSMS | 17 |
| GIDLSTYSMS | 18 |

HCVR CDR2

| | |
|---|---|
| TINTGGSAYY | 19 |
| NTGGSAYYA | 20 |
| TGGSAYY | 21 |
| IATINTGGSAYYA | 22 |
| NTGGSA | 23 |
| TINTGGSAYYA | 24 |
| INTGGSAYY | 25 |

HCVR CDR3

| | |
|---|---|
| CARTISADNLW | 26 |
| ATYFCARTIS | 27 |
| ADNLWGHGT | 28 |
| RTISADNL | 29 |
| TISADNLWGHG | 30 |
| ARTISADN | 31 |
| ARTISADNLWGHG | 32 |

Another non-limiting example of the monoclonal antibodies of the disclosure may comprise a light chain variable region (LCVR) having the following sequences, or functional fragments or variants thereof.

Light chain variable region (LCVR) nucleic acid sequence:
(SEQ ID NO: 6)
atggacacgagggcccccactcagctgctggggctcctgctgctctggctcccaggtgccacatttgcccaagtgct
gacccagactccatcctccgtgtctgcagctgtgggaagcacagtcaccatcagttgccagtccagtcagagtgtttat
agtgactacttatcttggtatcagcagaaaccaggacagcctcccaagcgcctgatatatgctgcatttactctggcatc
tggtgtcccatcgcggttcaaaggcagtggatctgagaaacagttcactctcaccatcagcgacctggagtgtgacg
atgctgccacttactactgtgcagccatttatagtaatgatatttgggctttcggcggagggaccgaggtggtcgtcaaa
ggtgatccagttgcacctactgtcctcatcttcccaccatcagcggaccaagtggccactggcaccgtgaccattgtct
gcgtggctaacaagtacttcccggacgtgaccgtgacctgggaagtcgacggaaccactcagaccactggtatcga
gaacagcaagacgccccagaactccgccgattgtacttataacctgtcctccacactgaccctcacctcgacccagta
caattcccacaaggagtacacttgcaaagtcacccagggaaccacttcagtggtgcagagcttcaaccggggggatt
gctga
LCVR amino acid sequence:
(SEQ ID NO: 7)
MDTRAPTQLLGLLLLWLPGATFAQVLTQTPSSVSAAVGSTVTISCQSSQS
VYSDYLSWYQQKPGQPPKRLIYAAFTLASGVPSRFKGSGSEKQFTLTISDL
ECDDAATYYCAAIYSNDIWAFGGGTEVVVKGDPVAPTVLIFPPSADQVAT
GTVTIVCVANKYFPDVTVTWEVDGTTQTTGIENSKTPQNSADCTYNLSST
LTLTSTQYNSHKEYTCKVTQGTTSVVQSFNRGDC

Based on the IMGT® prediction, this LCVR has the following CDRs.

LCVR CDR1:
(SEQ ID NO: 8)
QSVYSDY
LCVR CDR2:
(SEQ ID NO: 9)
AAF
LCVR CDR3:
(SEQ ID NO: 10)
AAIYSNDIWA

This LCVR may alternatively comprise the following CDRs:

| | |
|---|---|
| Sequence | SEQ ID NO: |

LCVR CDR1

| | |
|---|---|
| SSQSVYSDY | 33 |
| SQSVYSDYLSW | 34 |
| QSVYSDYLS | 35 |
| SVYSDYLS | 36 |
| VYSDYLSWY | 37 |
| YSDYLSWYQ | 38 |
| SDYLSWYQQ | 39 |

LCVR CDR2

| | |
|---|---|
| LIYAAFTLA | 40 |
| KRLIYAA | 41 |
| IYAAFTLASG | 42 |
| AFTLAASG | 43 |
| LIYAAFTL | 44 |
| YAAFTLASG | 45 |
| AAFTLASGV | 46 |
| YAAFT | 47 |
| IYAAFTL | 48 |

LCVR CDR3

| | |
|---|---|
| AATYYCAAIYSNDIWA | 49 |
| ATYYCAAIYSNDIWAF | 50 |
| TYYCAAIYSNDIWAFG | 51 |
| YYCAAIYSNDIWAFGG | 52 |
| YCAAIYSNDIWAFGGG | 53 |
| CAAIYSNDIWAFGGGT | 54 |
| AAIYSNDIWAFGGGTE | 55 |

In some embodiments, the monoclonal antibody of the disclosure comprises a HCVR comprising at least one CDR comprising at least about 70%, 75%, 80%, 85%, 90%, 95%, 96%, 97%, 98%, 99% or 100% sequence identity to the amino acid sequence of SEQ ID NO: 3, SEQ ID NO: 4, SEQ ID NO: 5, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: SEQ ID NO: 31 or SEQ ID NO: 32. In some embodiments, the monoclonal antibody comprises a HCVR comprising at least one CDR comprising at least about 70%, 75%, 80%, 85%, 90%, 95%, 96%, 97%, 98%, 99% or 100% sequence identity to the amino acid sequence of SEQ ID NO: 3, SEQ ID NO: 12, SEQ ID NO: 13, SEQ ID NO: 14, SEQ ID NO: 15, SEQ ID NO: 16, SEQ ID NO: 17 or SEQ ID NO: 18. In some embodiments, the monoclonal antibody comprises a HCVR comprising at least one CDR comprising at least about 70%, 75%, 80%, 85%, 90%, 95%, 96%, 97%, 98%, 99% or 100% sequence identity to the amino acid sequence of SEQ ID NO: 3. In some embodiments, the monoclonal antibody comprises a HCVR comprising at least one CDR comprising at least about 70%, 75%, 80%, 85%, 90%, 95%, 96%, 97%, 98%, 99% or 100% sequence identity to the amino acid sequence of SEQ ID NO: 4, SEQ ID NO: 19, SEQ ID NO: 20, SEQ ID NO: 21, SEQ ID NO: 22, SEQ ID NO: 23, SEQ ID NO: 24 or SEQ ID NO: 25. In some embodiments, the monoclonal antibody comprises a HCVR comprising at least one CDR comprising at least about 70%, 75%, 80%, 85%, 90%, 95%, 96%, 97%, 98%, 99% or 100% sequence identity to the amino acid sequence of SEQ ID NO: 4. In some embodiments, the monoclonal antibody comprises a HCVR comprising at least one CDR comprising at least about 70%, 75%, 80%, 85%, 90%, 95%, 96%, 97%, 98%, 99% or 100% sequence identity to the amino acid sequence of SEQ ID NO: 5, SEQ ID NO: 26, SEQ ID NO: 27, SEQ ID NO: 28, SEQ ID NO: 29, SEQ ID NO: 30, SEQ ID NO: SEQ ID NO: 31 or SEQ ID NO: 32. In some embodiments, the monoclonal antibody comprises a HCVR comprising at least one CDR comprising at least about 70%, 75%, 80%, 85%, 90%, 95%, 96%, 97%, 98%, 99% or 100% sequence identity to the amino acid sequence of SEQ ID NO: 5. In some embodiments, the monoclonal antibody comprises a HCVR comprising a first CDR comprising SEQ ID NO: 3 or a variant thereof and a second CDR comprising SEQ ID NO: 4 or a variant thereof. In some embodiments, the monoclonal antibody comprises a HCVR comprising a first CDR comprising SEQ ID NO: 3 or a variant thereof and a second CDR comprising SEQ ID NO: 5 or a variant thereof. In some embodiments, the monoclonal antibody comprises a HCVR comprising a first CDR comprising SEQ ID NO: 4 or a variant thereof and a second CDR comprising SEQ ID NO: 5 or a variant thereof. In some embodiments, the monoclonal antibody comprises a HCVR comprising a first CDR comprising SEQ ID NO: 3 or a variant thereof, a second CDR comprising SEQ ID NO: 4 or a variant thereof, and a third CDR comprising the amino acid sequence of SEQ ID NO: 5 or a variant thereof. As used herein, SEQ ID NO: 12, SEQ ID NO: 13, SEQ ID NO: 14, SEQ ID NO: 15, SEQ ID NO: 16, SEQ ID NO: 17 and SEQ ID NO: 18 are considered to be variants of SEQ ID NO: 3; SEQ ID NO: 19, SEQ ID NO: 20, SEQ ID NO: 21, SEQ ID NO: 22, SEQ ID NO: 23, SEQ ID NO: 24 and SEQ ID NO: 25 are considered to be variants of SEQ ID NO: 4; and SEQ ID NO: 26, SEQ ID NO: 27, SEQ ID NO: 28, SEQ ID NO: 29, SEQ ID NO: 30, SEQ ID NO: SEQ ID NO: 31 and SEQ ID NO: 32 are considered to be variants of SEQ ID NO: 5. In some embodiments, the monoclonal antibody comprises a HCVR comprising at least about 70%, 75%, 80%, 85%, 90%, 95%, 96%, 97%, 98% or 99% sequence identity to SEQ ID NO: 2. In some embodiments, the monoclonal antibody comprises a HCVR comprising SEQ ID NO: 2.

In some embodiments, the monoclonal antibody of the disclosure comprises a LCVR comprising at least one CDR comprising at least about 70%, 75%, 80%, 85%, 90%, 95%, 96%, 97%, 98%, 99% or 100% sequence identity to the amino acid sequence of SEQ ID NO: 8, SEQ ID NO: 9, SEQ ID NO: 10, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54 or SEQ ID NO: 55. In some embodiments, the monoclonal antibody comprises a LCVR comprising a CDR comprising at least about 70%, 75%, 80%, 85%, 90%, 95%, 96%, 97%, 98%, 99% or 100% sequence identity to SEQ ID NO: 8, SEQ ID NO: 33, SEQ ID NO: 34, SEQ ID NO: 35, SEQ ID NO: 36, SEQ ID NO: 37, SEQ ID NO: 38 or SEQ ID NO: 39. In some embodiments, the monoclonal antibody comprises a LCVR comprising a CDR comprising at least about 70%, 75%, 80%, 85%, 90%, 95%, 96%, 97%, 98%, 99% or 100% sequence identity to SEQ ID NO: 8. In some embodiments, the monoclonal antibody comprises a LCVR comprising a CDR comprising at least about 70%, 75%, 80%, 85%, 90%, 95%, 96%, 97%, 98%, 99% or 100% sequence identity to SEQ ID NO: 9, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 or SEQ ID NO: 48. In some embodiments, the monoclonal antibody comprises a LCVR comprising a CDR comprising at least about 70%, 75%, 80%, 85%, 90%, 95%, 96%, 97%, 98%, 99% or 100% sequence identity to SEQ ID NO: 9. In some embodiments, the monoclonal antibody comprises a LCVR comprising a CDR comprising at least about 70%, 75%, 80%, 85%, 90%, 95%, 96%, 97%, 98%, 99% or 100% sequence identity to SEQ ID NO: 10, SEQ ID NO: 49, SEQ ID NO: 50, SEQ ID NO: 51, SEQ ID NO: 52, SEQ ID NO: 53, SEQ ID NO: 54 or SEQ ID NO: 55. In some embodiments, the monoclonal antibody comprises a LCVR comprising a CDR comprising at least about 70%, 75%, 80%, 85%, 90%, 95%, 96%, 97%, 98%, 99% or 100% sequence identity to SEQ ID NO: 10. In some embodiments, the monoclonal antibody comprises a LCVR comprising a first CDR comprising SEQ ID NO: 8 or a variant thereof and a second CDR comprising SEQ ID NO: 9 or a variant thereof. In some embodiments, the monoclonal antibody comprises a LCVR comprising a first CDR comprising SEQ ID NO: 8 or a variant thereof and a second CDR comprising SEQ ID NO: 10 or a variant thereof. In some embodiments, the monoclonal antibody comprises a LCVR comprising a first CDR comprising SEQ ID NO: 9 or a variant thereof and a second CDR comprising SEQ ID NO: 10 or a variant thereof. In some embodiments, the monoclonal antibody comprises a LCVR comprising a first CDR comprising SEQ ID NO: 8 or a variant thereof, a second CDR comprising SEQ ID NO: 9 or a variant thereof, and a third CDR comprising SEQ ID NO: 10 or a variant thereof. As used herein, SEQ ID NO: 33, SEQ ID NO: 34, SEQ ID NO: 35, SEQ ID NO: 36, SEQ ID NO: 37, SEQ ID NO: 38 and SEQ ID NO: 39 are considered to be variants of SEQ ID NO: 8; SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 and SEQ ID NO: 48 are considered to be variants of SEQ ID NO: 9; and SEQ ID NO: 49, SEQ ID NO: 50, SEQ ID NO: 51, SEQ ID NO: 52, SEQ ID NO: 53, SEQ ID NO: 54 and SEQ ID NO: 55 are considered to be variants of SEQ ID NO: 10. In some embodiments, the monoclonal antibody comprises a LCVR comprising at least about 70%, 75%, 80%, 85%, 90%, 95%, 96%, 97%, 98% or 99% sequence identity to SEQ ID NO: 7. In some embodiments, the monoclonal antibody comprises a LCVR comprising SEQ ID NO: 7.

In some embodiments, the monoclonal antibody of the disclosure comprises a HCVR comprising at least one CDR chosen from SEQ ID NO: 3, SEQ ID NO: 4 or SEQ ID NO: 5, or a variant thereof, and a LCVR comprising at least one CDR chosen from SEQ ID NO: 8, SEQ ID NO: 9 or SEQ ID NO: 10, or a variant thereof. In some embodiments, the monoclonal antibody of the disclosure comprises a HCVR comprising at least two CDR chosen from SEQ ID NO: 3, SEQ ID NO: 4 or SEQ ID NO: 5, or a variant thereof, and a LCVR comprising at least two CDR chosen from SEQ ID NO: 8, SEQ ID NO: 9 or SEQ ID NO: 10, or a variant thereof. In some embodiments, the monoclonal antibody of the disclosure comprises a HCVR comprising SEQ ID NO: 3, SEQ ID NO: 4 and SEQ ID NO: 5, or their respective variant thereof, and a LCVR comprising SEQ ID NO: 8, SEQ ID NO: 9 and SEQ ID NO: 10, or their respective variant thereof. In some embodiments, the monoclonal antibody of the disclosure comprises a HCVR comprising at least about 70%, 75%, 80%, 85%, 90%, 95%, 96%, 97%, 98% or 99% sequence identity to SEQ ID NO: 2 and a LCVR comprising at least about 70%, 75%, 80%, 85%, 90%, 95%, 96%, 97%, 98% or 99% sequence identity to SEQ ID NO: 7. In some embodiments, the monoclonal antibody of the disclosure comprises a HCVR comprising SEQ ID NO: 2 and a LCVR comprising SEQ ID NO: 7.

An antigen-binding fragment of the monoclonal antibody of the disclosure may incorporate any of the above CDRs as part of a larger polypeptide chain, may covalently link any of the above CDRs to another polypeptide chain, or may incorporate any of the above CDRs noncovalently. The CDRs permit the antigen-binding fragment to specifically bind to a particular antigen of interest, such as PIF peptide in the instant case.

In some embodiments, the monoclonal antibody, or the antigen-binding fragment thereof, of the disclosure may further comprise a heavy chain constant region. In some embodiments, the monoclonal antibody, or the antigen-binding fragment thereof, may further comprise a light chain constant region. In some embodiments, the monoclonal antibody, or the antigen-binding fragment thereof, may further comprise a heavy chain constant region and a light chain constant region.

The monoclonal antibody of the present disclosure may be of an IgG class. The monoclonal antibody of the present disclosure may further be an IgG1 isotype.

In some embodiments, the monoclonal antibody, or the antigen-binding fragment thereof, of the present disclosure binds to a preimplantation factor (PIF), or an immunogenic fragment or epitope thereof. The full-length PIF is a 15-amino acid peptide having the sequence of MVRIKPGSANKPSDD (SEQ ID NO: 11). An immunogenic fragment or epitope of the PIF peptide can consist of a single contiguous sequence of 3 or more (e.g., 3, 4, 5, 6, 7, 8, 9, 10, 11, 12, 13 or 14) amino acids of the full-length PIF, such as 14-mer, 13-mer, 12-mer, 11-mer, 10-mer, 9-mer, 8-mer, 7-mer or even less than 7 amino acid residues. Alternatively, the immunogenic fragment or epitope can consist of a plurality of non-contiguous amino acids (or amino acid sequences) of PIF. In some embodiments, the monoclonal antibody, or the antigen-binding fragment thereof, of the present disclosure is specific to a full-length PIF. “Specific to a full length PIF,” as used herein, means that the monoclonal antibody, or the antigen-binding fragment thereof, binds specifically to the full length of PIF, but not derivatives of PIF in different length. In some embodiments therefore, the monoclonal antibody, or the antigen-binding fragment thereof, of the present disclosure is specific to the PIF comprising the amino acid sequence of SEQ ID NO: 11.

In some embodiments, the monoclonal antibody, or the antigen-binding fragment thereof, of the disclosure binds to PIF, or an immunogenic fragment or epitope thereof, with greater affinity than it binds to an unrelated control protein. In some embodiments, the monoclonal antibody, or the antigen-binding fragment thereof, of the disclosure binds to PIF, or an immunogenic fragment or epitope thereof, with an affinity that is at least about 50, about 100, about 250, about 500, about 1000, or about 10,000 times greater than the affinity for a control protein. In some embodiments, the monoclonal antibody, or the antigen-binding fragment thereof, of the disclosure have a binding affinity for PIF, or an immunogenic fragment or epitope thereof, of less than or equal to about 1×10⁻⁷ M, less than or equal to about 1×10⁻⁸ M, less than or equal to about 1×10⁻⁹ M, less than or equal to about 1×10⁻¹⁰ M, less than or equal to about 1×10⁻¹¹ M, or less than or equal to about 1×10⁻¹² M.

Affinity may be determined by an affinity ELISA assay. In some embodiments, affinity may be determined by a BIAcore assay. In other embodiments, affinity may be determined by a kinetic method. In some embodiments, affinity may be determined by an equilibrium/solution method. All these methods are well known and routinely performed by the skilled in the art.

In some embodiments, the monoclonal antibody, or the antigen-binding fragment thereof, of the present disclosure is a rabbit monoclonal antibody.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype (Lantto et al., 2002, Methods Mol. Biol. 178:303-16).

The monoclonal antibody, or the antigen-binding fragment thereof, of the disclosure can comprise one or more amino acid substitutions, insertions and/or deletions in the framework and/or CDR regions of the heavy and light chain variable domains. Such mutations can be readily ascertained by comparing the amino acid sequences disclosed herein to sequences available from, for example, public antibody sequence databases. Once obtained, antibodies and antigen-binding fragments that contain one or more mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The disclosure also includes monoclonal antibodies that are capable of binding to a PIF comprising variants of the HCVR, LCVR, and/or CDR amino acid sequences disclosed herein having one or more conservative substitutions. For example, the present disclosure includes anti-PIF antibodies having HCVR, LCVR, and/or CDR amino acid sequences with, e.g., about 10 or fewer, about 8 or fewer, about 6 or fewer, about 4 or fewer, etc. conservative amino acid substitutions relative to the HCVR, LCVR, and/or CDR amino acid sequences disclosed herein.

The disclosure provides monoclonal antibodies structurally characterized by the amino acid sequences of their variable domain regions. However, the amino acid sequences can undergo some changes while retaining their high degree of binding to their specific targets. More specifically, many amino acids in the variable domain region can be changed with conservative substitutions and it is predictable that the binding characteristics of the resulting antibody will not differ from the binding characteristics of the wild type antibody sequence. There are many amino acids in an antibody variable domain that do not directly interact with the antigen or impact antigen binding and are not critical for determining antibody structure. For example, a predicted nonessential amino acid residue in any of the disclosed antibodies is preferably replaced with another amino acid residue from the same class. Methods of identifying amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997), all of which are incorporated by reference in their entireties herein). Near et al. (Mol. Immunol. 30:369-377, 1993) explains how to impact or not impact binding through site-directed mutagenesis. Near et al. only mutated residues that they thought had a high probability of changing antigen binding. Most had a modest or negative effect on binding affinity (Near et al. Table 3) and binding to different forms of digoxin (Near et al. Table 2).

A conservative modification or functional equivalent of a peptide, polypeptide, or protein disclosed in this disclosure (e.g., the hinge region or a heavy chain having the hinge region) refers to a polypeptide derivative of the peptide, polypeptide, or protein, e.g., a protein having one or more point mutations, insertions, deletions, truncations, a fusion protein, or a combination thereof. It retains substantially the activity of the parent peptide, polypeptide, or protein (such as those disclosed in this disclosure). In general, a conservative modification or functional equivalent is at least about 60% (e.g., any number between 60% and 100%, inclusive, e.g., 60%, 70%, 75%, 80%, 85%, 90%, 95%, 96%, 97%, 98%, and 99%) identical to a parent sequence (e.g., any one of the sequences disclosed herein).

In some embodiments, the substitutions made within a heavy or light chain that is at least about 95% identical (or at least about 96% identical, or at least about 97% identical, or at least about 98% identical, or at least about 99% identical) to a parent sequence, such as any one of the sequences disclosed herein, are conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Exemplary conservative substitutions are set out in Tables A, B and C below.

TABLE A
Conservative Substitutions I

| Side Chain Characteristics | Amino Acid |
|---|---|
| Aliphatic | |
| Non-polar | G A P I L V F |
| Polar - uncharged | C S T M N Q |
| Polar - charged | D E K R |
| Aromatic | H F W Y |
| Other | N Q D E |

TABLE B
Conservative Substitutions II

| Side Chain Characteristic | Amino Acid |
|---|---|
| Non-polar (hydrophobic) | |
| Aliphatic: | A L I V P |
| Aromatic: | F W Y |
| Sulfur-containing: | M |
| Borderline: | G Y |
| Uncharged-polar | |
| Hydroxyl: | S T Y |
| Amides: | N Q |
| Sulfhydryl: | C |
| Borderline: | G Y |
| Positively Charged (Basic): | K R H |
| Negatively Charged (Acidic): | D E |

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4: 11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm, which has been incorporated into the GAP program in the GCG software package (available at gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present disclosure can further be used as a “query sequence” to perform a search against public databases to, for example, identify related sequences. Such searches can be performed using the)(BLAST program (version 2.0) of Altschul, et al. (1990) J. Mol. Biol. 215:403-10. BLAST protein searches can be performed with the)(BLAST program, score=50, wordlength=3 to obtain amino acid sequences homologous to the molecules of the disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g.,)(BLAST and NBLAST) can be used (see ncbi.nlm.nih.gov).

Other modifications of the antibody are contemplated herein. For example, the antibody can be linked to one of a variety of nonproteinaceous polymers, for example, polyethylene glycol, polypropylene glycol, polyoxyalkylenes, or copolymers of polyethylene glycol and polypropylene glycol. The antibody also can be entrapped in microcapsules prepared, for example, by coacervation techniques or by interfacial polymerization (for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules), or in macroemulsions. Such techniques are disclosed in, for example, Remington's Pharmaceutical Sciences, 16th edition, Oslo, A., Ed., (1980).

Variant antibodies and salts thereof also are included within the scope of the disclosure. Variants of the sequences recited in the application also are included within the scope of the disclosure. Further variants of the antibody sequences having improved affinity can be obtained using methods known in the art and are included within the scope of the disclosure. For example, amino acid substitutions can be used to obtain antibodies with further improved affinity. Alternatively, codon optimization of the nucleotide sequence can be used to improve the efficiency of translation in expression systems for the production of the antibody. Variants may include non-natural amino acids up to a certain percentage. In some embodiments, the antibody comprises a variant amino acid sequence comprising about 1, 2, 3, 4, 5, 6, 7, 8, 9, 10, 11, 12, 13, 14, 15, 16, 17, 18, 19, 20 or more percent of non-natural amino acids.

In some embodiments, the monoclonal antibodies that are capable of binding to a PIF can be fully human (non-naturally occurring) antibodies. Methods for generating monoclonal antibodies, including fully human monoclonal antibodies are known in the art. Any such known methods can be used in the context of the present disclosure to make human antibodies that specifically bind to human PIF.

Methods for generating antibodies are known in the art and/or described in Harlow and Lane (editors) Antibodies: A Laboratory Manual, Cold Spring Harbor Laboratory, (1988). Generally, in such methods, PIF or a region thereof or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies.

For the production of monoclonal antibodies, any one of a number of known techniques may be used, such as, for example, the procedure exemplified in U.S. Pat. No. 4,196,265 or Harlow and Lane (1988), supra. For example, a suitable animal is immunized with an immunogen under conditions sufficient to stimulate antibody producing cells. Rodents such as rabbits, mice and rats are exemplary animals. Mice genetically-engineered to express human antibodies but do not express murine antibodies, for example, can also be used to generate an antibody of the present disclosure (e.g., as described in WO 2002/066630, the content of which is incorporated by reference herein).

Following immunization, somatic cells with the potential for producing antibodies, specifically B lymphocytes (B cells), are selected for use in the monoclonal antibody generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen. Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemstry and/or immunoassay (e.g. radioimmunoassay, enzyme immunoassay, cytotoxicity assay, plaque assay, dot immunoassay, and the like). Alternatively, ABL-MYC technology (NeoClone, Madison Wis. 53713, USA) is used to produce cell lines secreting monoclonal antibodies (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793.

The monoclonal antibody of the disclosure may also be a synthetic antibody. For example, the antibody is a chimeric antibody, a humanized antibody, a human antibody, synhumanized antibody, primatized antibody or a de-immunized antibody.

Accordingly, the disclosure also relates to a nucleic acid molecule encoding any of the disclosed monoclonal antibodies. In some embodiments, such nucleic acid molecules encode polypeptides comprising at least one of SEQ ID NO: 3, SEQ ID NO: 4, SEQ ID NO: 5, SEQ ID NO: 8, SEQ ID NO: 9 and SEQ ID NO: 10, or their respective variant comprising at least about 70%, 75%, 80%, 85%, 90%, 95%, 96%, 97%, 98% or 99% sequence identity thereto. In some embodiments, the nucleic acid molecule of the disclosure encodes a polypeptide comprising at least two of SEQ ID NO: 3, SEQ ID NO: 4, SEQ ID NO: 5, SEQ ID NO: 8, SEQ ID NO: 9 and SEQ ID NO: 10, or their respective variant comprising at least about 70%, 75%, 80%, 85%, 90%, 95%, 96%, 97%, 98% or 99% sequence identity thereto. In some embodiments, the nucleic acid molecule encodes a polypeptide comprising at least three of SEQ ID NO: 3, SEQ ID NO: 4, SEQ ID NO: 5, SEQ ID NO: 8, SEQ ID NO: 9 and SEQ ID NO: 10, or their respective variant comprising at least about 70%, 75%, 80%, 85%, 90%, 95%, 96%, 97%, 98% or 99% sequence identity thereto. In some embodiments, the nucleic acid molecule encodes a polypeptide comprising at least four of SEQ ID NO: 3, SEQ ID NO: 4, SEQ ID NO: 5, SEQ ID NO: 8, SEQ ID NO: 9 and SEQ ID NO: 10, or their respective variant comprising at least about 70%, 75%, 80%, 85%, 90%, 95%, 96%, 97%, 98% or 99% sequence identity thereto. In some embodiments, the nucleic acid molecule encodes a polypeptide comprising at least five of SEQ ID NO: 3, SEQ ID NO: 4, SEQ ID NO: 5, SEQ ID NO: 8, SEQ ID NO: 9 and SEQ ID NO: 10, or their respective variant comprising at least about 70%, 75%, 80%, 85%, 90%, 95%, 96%, 97%, 98% or 99% sequence identity thereto. In some embodiments, the nucleic acid molecule encodes a polypeptide comprising SEQ ID NO: 3, SEQ ID NO: 4, SEQ ID NO: 5, SEQ ID NO: 8, SEQ ID NO: 9 and SEQ ID NO: 10, or their respective variant comprising at least about 70%, 75%, 80%, 85%, 90%, 95%, 96%, 97%, 98% or 99% sequence identity thereto.

In some embodiments, the nucleic acid molecule of the disclosure encodes a polypeptide comprising at least about 70%, 75%, 80%, 85%, 90%, 95%, 96%, 97%, 98% or 99% sequence identity to SEQ ID NO: 2. In some embodiments, the nucleic acid molecule encodes a polypeptide comprising SEQ ID NO: 2. In some embodiments, the nucleic acid molecule of the disclosure encodes a polypeptide comprising at least about 70%, 75%, 80%, 85%, 90%, 95%, 96%, 97%, 98% or 99% sequence identity to SEQ ID NO: 7. In some embodiments, the nucleic acid molecule encodes a polypeptide comprising SEQ ID NO: 7. In some embodiments, the nucleic acid molecule encodes a polypeptide comprising at least about 70%, 75%, 80%, 85%, 90%, 95%, 96%, 97%, 98% or 99% sequence identity to SEQ ID NO: 2 and SEQ ID NO: 7. In some embodiments, the nucleic acid molecule encodes a polypeptide comprising SEQ ID NO: 2 and SEQ ID NO: 7.

In some embodiments, the nucleic acid molecule of the disclosure comprises at least about 70%, 75%, 80%, 85%, 90%, 95%, 96%, 97%, 98% or 99% sequence identity to SEQ ID NO: 1. In some embodiments, the nucleic acid molecule comprises SEQ ID NO: 1. In some embodiments, the nucleic acid molecule comprises at least about 70%, 75%, 80%, 85%, 90%, 95%, 96%, 97%, 98% or 99% sequence identity to SEQ ID NO: 6. In some embodiments, the nucleic acid molecule comprises SEQ ID NO: 6. In some embodiments, the nucleic acid molecule comprises at least about 70%, 75%, 80%, 85%, 90%, 95%, 96%, 97%, 98% or 99% sequence identity to SEQ ID NO: 1 and SEQ ID NO: 6. In some embodiments, the nucleic acid molecule comprises SEQ ID NO: 1 and SEQ ID NO: 6.

Recombinant Vectors and Host Cells

The disclosure further provides vectors, such as expression vectors, comprising any of the disclosed nucleic acid molecules. A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector, wherein additional DNA segments can be ligated into the viral genome. In some embodiments therefore, the vector of the disclosure is a plasmid. In other embodiments, the vector of the disclosure is a viral vector.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, e.g., expression vectors, are capable of directing the expression of genes to which they are operably linked. In general, expression vectors of utility in recombinant DNA techniques are often in the form of plasmids (vectors). However, the disclosure is intended to include such other forms of expression vectors, such as viral vectors (e.g., replication defective retroviruses, adenoviruses and adeno-associated viruses), which serve equivalent functions.

The recombinant expression vectors of the disclosure comprise a nucleic acid in a form suitable for expression of any of the disclosed nucleic acids in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). In addition, the regulatory sequence is optimized based on the host cell characteristics, i.e., transcription factors.

The term “regulatory sequence” is intended to include promoters, enhancers and other expression control elements (e.g., polyadenylation signals). Such regulatory sequences are described in, for example, Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the disclosure can be introduced into host cells to thereby produce antibodies encoded by nucleic acids as described herein.

In some embodiments, the disclosure further provides methods for the generation and use of host cell expression systems for the production of antibodies or antibody fragments or fusion proteins which include antibody fragments that are encoded by any of the nucleic acids disclosed herein. Any types of non-human cells, such as mammalian cells, plant cells, insect cells, bacterial cells or yeast are encompassed in the present disclosure. In some embodiments, mammalian cells are used as host cell systems transfected with recombinant plasmid DNA or cosmid DNA expression vectors containing any of the disclosed nucleic acids. In some embodiments, CHO cells, BEM cells, NS0 cells, or SP2/0 cells, or alternatively, hybridoma cells are used as host cell systems. For the production of antibodies in large amounts, hybridoma, a type of hybrid cell, can be used. Thus, in some embodiments, the non-human cell of the disclosure is a hybridoma. In some embodiments, other eukaryotic host cell systems may be contemplated, including but not limited to, yeast cells transformed with recombinant yeast expression vectors containing any of the disclosed nucleic acids; insect cell systems infected with recombinant virus expression vectors (e.g., baculovirus) containing any of the disclosed nucleic acids; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing any of the disclosed nucleic acids; animal cell systems infected with recombinant virus expression vectors (e.g., adenovirus, vaccinia virus) containing any of the disclosed nucleic acids or including cell lines engineered to contain multiple copies of any of the disclosed nucleic acids either stably amplified (CHO/dhfr) or unstably amplified in double-minute chromosomes (e.g., murine cell lines). Methods of using such non-human cells for protein expression are known in the art and routinely used by one skilled person. In some embodiments, the non-human cells of the disclosure are actively secreting the produced antibody and display the antibody on the surface of the cells.

The expression of antibody in the disclosed host cell expression systems may be stable or transient. Stable expression typically achieves more reproducible results and is more amenable to large scale production. Rather than using expression vectors which contain viral origins of replication, host cells can be transformed with the respective coding nucleic acids controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows selection of cells which have stably integrated the plasmid into their chromosomes and grow to form foci which in turn can be cloned and expanded into cell lines. In some embodiments, the expression of antibody in the disclosed host cell expression systems is stable expression. In other embodiments, the expression of antibody in the disclosed host cell expression systems is transient expression.

Systems

Also provided in the disclosure are systems comprising any of the disclosed monoclonal antibodies. In some embodiments, the system of the disclosure comprises: a) one or a plurality of any of the disclosed monoclonal antibodies; b) a solid support onto which the one or plurality of monoclonal antibodies are immobilized; and c) at least one substrate. The system of the disclosure can be used to detect and/or determine the presence, absence, and/or biologically significant quantity of a PIF in a sample. In some embodiments, the system of the disclosure can be used to perform any of the methods described herein. In some embodiments, the system can be used to detect embryo fitness for implantation. In some embodiments, the system can be used to detect a PIF in an embryonic culture medium. In some embodiments, the system can be used to detect protein that binds to a PIF. In some embodiments, the system can be used to determine suitability of one or more embryos for in vitro fertilization.

In some embodiments, the system of the disclosure is a fluorescence-based system to detect and/or determine the presence, absence, and/or biologically significant quantity of a PIF in a sample. In some embodiments therefore, the disclosed system further comprises a fluorimeter or spectrophotometer. In some embodiments, the at least one substrate comprised in the system of the disclosure are fluorogenic, fluorescent, or chemiluminescent fluorescently labeled. One skilled in the art will recognize that many strategies can be used for labeling target molecules to enable their detection or discrimination in a mixture of particles. The labels may be attached by any known means, including methods that utilize non-specific or specific interactions of label and target. Labels may provide a detectable signal or affect the mobility of the particle in an electric field.

In some embodiments, the substrate of the disclosure is labeled by attaching the substrate to a fluorescent moiety. In some embodiments, the fluorescent moiety used to label the substrate of the disclosure is a fluorescent dye molecule. In some embodiments, the fluorescent dye molecule is or comprises one or more fluorescent dyes, including but not limited to fluorescein, rhodamine, Alexa Fluors, DyLight fluors, ATTO Dyes, or any analogs or derivatives thereof. In some embodiments, the fluorescent dye molecules include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red). In some embodiments, the fluorescent dye molecules include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bi s(phenylethynyl)naphthacene; 9, 10-Bi s(phenyl ethynyl)anthrac ene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl)anthracene; 2-Chloro-9,10-bis(phenylethynyl)anthracene; 2-Chloro-9, 10-diphenylanthracene; Coumarin; Cyanine dyes (e.g., Cyanine such as Cy3 and Cy5, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Optical brightener, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris(bathophenanthroline disulfonate)ruthenium(II), Texas Red, TSQ, Umbelliferone, or Yellow fluorescent protein. In some embodiments, the fluorescent dye molecules include but are not limited to Alexa Fluor family of fluorescent dyes (Molecular Probes, Oregon). Alexa Fluor dyes are widely used as cell and tissue labels in fluorescence microscopy and cell biology. The excitation and emission spectra of the Alexa Fluor series cover the visible spectrum and extend into the infrared. The individual members of the family are numbered according roughly to their excitation maxima (in nm). Certain Alexa Fluor dyes are synthesized through sulfonation of coumarin, rhodamine, xanthene (such as fluorescein), and cyanine dyes. Sulfonation makes Alexa Fluor dyes negatively charged and hydrophilic. Alexa Fluor dyes are more stable, brighter, and less pH-sensitive than common dyes (e.g. fluorescein, rhodamine) of comparable excitation and emission, and to some extent the newer cyanine series. In some embodiments, the fluorescent dye molecules include but are not limited AlexaFluor 350, AlexaFluor 405, AlexaFluor 430, AlexaFluor 488, AlexaFluor 500, AlexaFluor 514, AlexaFluor 532, AlexaFluor 546, AlexaFluor 555, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750, or AlexaFluor 790. n some embodiments, the dye molecules are AlexaFluor647 dye molecules. In some embodiments, the fluorescent dye molecules include but are not limited to the DyLight Fluor family of fluorescent dyes (Dyomics and Thermo Fisher Scientific). In some embodiments, the fluorescent dye molecules include but are not limited DyLight 350, DyLight 405, DyLight 488, DyLight 550, DyLight 594, DyLight 633, DyLight 650, DyLight 680, DyLight 755, or DyLight-800.

In some embodiments, the fluorescent moiety used to label the substrate of the disclosure comprises a first type and a second type of fluorescent dye molecules. In some embodiments, the first type and second type of fluorescent dye molecules have different emission spectra. In some embodiments, the ratio of the number of first type to second type of fluorescent dye molecule is about 4:1, 3:1, 2:1, 1:1, 1:2, 1:3 or 1:4.

In some embodiments, the substrate of the disclosure is capable of binding to the one or plurality of monoclonal antibodies comprised in the system of the disclosure. In some embodiments, the substrate is a preimplantation factor (PIF), or an immunogenic fragment or epitope thereof. In some embodiments, the substrate comprises at least about 70%, 75%, 80%, 85%, 90%, 95%, 96%, 97%, 98% or 99% sequence identity to SEQ ID NO: 11, or a functional fragment thereof. In some embodiments, the substrate comprises the amino acid sequence of SEQ ID NO: 11. In some embodiments, the probe is a PIF peptide, a functional fragment thereof, or an immunogenic fragment or epitope thereof, that is fluorescently labeled with one or a plurality of the aforementioned fluorescent dye molecules. In some embodiments, the fluorescent dye molecule is covalently or non-covalently bound to the PIF peptide at the N-terminus. In some embodiments, the fluorescent dye molecule is covalently or non-covalently bound to the PIF peptide at the C-terminus.

In some embodiments, the monoclonal antibody comprised in the system of the disclosure is immobilized, adsorbed, bound, or otherwise associated to a solid support or a bead, such as a magnetic bead. Solid supports can be tissue culture plates, microarray plates, plastic or polystyrene multiwall plates, or other plastic elements with one or a plurality of reaction vessels (chambers or compartments). PIF peptides contained in a sample can be exposed to one or a plurality of monoclonal antibodies within one or a plurality of reaction vessels on the solid support in the presence of a defined amount of labeled substrate for a time period sufficient to cause the binding between PIF peptides and antibodies and/or labeled substrate and antibodies. In some embodiments, binding products between labeled substrate and antibodies can be detected in solution or within the reaction vessel after exposure of the reaction vessel to one or a plurality of chemical stimuli for a chemiluminescent probe or visible or non-visible light that is capable of activated the electronic state of a fluorescent probe. As the disclosed systems detect the presence, absence, and/or biologically significant quantity of PIF peptides in a sample primarily based on a competitive assay theory, the more PIF peptides in a sample will theoretically cause less binding between labeled substrate and antibodies, and thus less fluorescent signal being detected. In some embodiments, the monoclonal antibodies disclosed herein can be immobilized or bound onto the surface of a solid support where one or more of FRET analysis, Raman spectroscopy, mass spectroscopy, fluorescent microscopy or absorbance of light may be performed. In some embodiments, the monoclonal antibodies disclosed herein can be immobilized or bound onto the surface of a solid support where one or more of SPR spectroscopy, NMR spectroscopy, X-ray crystallography and electron microscopy (EM) may be performed.

Any type of solid support typically used by one of ordinary skill in the art may be used. In some embodiments, the solid support is a chip. In some embodiments, the solid support is a slide. In some embodiments, the solid support is a microarray chip or slide. In some embodiments, the solid support is a bead, such as a magnetic bead. In some embodiments, the solid support is a stripe. In some embodiments, the solid support is a membrane. In some embodiments, the solid support is a petri dish or polysterene plate. In some embodiments, the solid support is a multiwell plate, including but not limited to, 12-well, 24-well, 36-well, 48-well, 96-well, 192-well, and 384-well plate. Suitable methods for immobilizing a subject antibody onto a solid support are well known and include, but are not limited to ionic, hydrophobic, covalent interactions and the like.

In some embodiments, the monoclonal antibody comprised in the system of the disclosure is immobilized onto the solid support via another antibody that recognizes the monoclonal antibody of the disclosure as an antigen. In some embodiments, the monoclonal antibody of the disclosure is a rabbit monoclonal antibody and it is immobilized onto the solid support via one or a plurality of mammalian rabbit anti-human antibodies. In some embodiments, one or plurality of mammalian-rabbit antibodies are goat anti-rabbit antibodies. In some embodiments therefore, the monoclonal antibody comprised in the system of the disclosure is a rabbit monoclonal antibody. In some embodiments, the system of the disclosure further comprises one or a plurality of mammalian rabbit, anti-human PIF antibodies. In some embodiments, the system further comprises one or a plurality of goat anti-human PIF antibodies.

Any sample which contains or suspected of containing PIF peptides can be used in the disclosed systems. When used in conjunction with any of the methods disclosed herein, the sample can be obtained from a spent embryo culture medium used for culturing one or a plurality of embryos. In some embodiments, the sample embryo culture medium used for culturing one or a plurality of embryos at about Day 3 after culturing the one or plurality of embryos. In some embodiments, the sample is embryo culture medium used for culturing one or a plurality of embryos at about Day 4 after culturing the one or plurality of embryos. In some embodiments, the sample is embryo culture medium used for culturing one or a plurality of embryos at about Day 5 after culturing the one or plurality of embryos. In some embodiments, the sample an embryo culture medium used for culturing one or a plurality of embryos at about Day 6 after culturing the one or plurality of embryos. In some embodiments, the sample is an embryo culture medium used for culturing one or a plurality of embryos at about Day 7 after culturing the one or plurality of embryos. In some embodiments, the sample is an embryo culture medium used for culturing one or a plurality of embryos at about Day 8 after culturing the one or plurality of embryos. In some embodiments, the sample is embryo culture medium used for culturing one or a plurality of embryos at about Day 9 after culturing the one or plurality of embryos. In some embodiments, the sample is embryo culture medium used for culturing one or a plurality of embryos at about Day after culturing the one or plurality of embryos.

In some embodiments, the sample is embryo culture medium from medium exposed to one or a plurality of embryos at about Day 3 to about Day 10 after culturing the one or plurality of embryos. In some embodiments, the sample is sample is embryo culture medium from medium exposed to one or a plurality of embryos at about Day 3 to about Day 7 after culturing the one or plurality of embryos. In some embodiments, the sample sample is embryo culture medium from medium exposed to one or a plurality of embryos at about Day 3 to about Day 6 after culturing the one or plurality of embryos. In some embodiments, the sample is sample is embryo culture medium from medium exposed to one or a plurality of embryos at about Day 3 to about Day 5 after culturing the one or plurality of embryos. In some embodiments, the sample sample is embryo culture medium from medium exposed to one or a plurality of embryos at about Day 3 to about Day 6 after culturing the one or plurality of embryos. In some embodiments, the sample is embryo culture medium from medium exposed to one or a plurality of embryos at about Day 4 to about Day 6 after culturing the one or plurality of embryos.

In some embodiments, the embryo culture medium was exposed to a single embryo. In some embodiments, the embryo culture medium was exposed to about 2 human embryos. In some embodiments, the embryo culture medium was exposed to about 3 or fewer human embryos. In some embodiments, the embryo culture medium was exposed to about 4 or fewer human embryos. In some embodiments, the embryo culture medium was exposed to about 5 or fewer human embryos. In some embodiments, the embryo culture medium was exposed to about 5 or more human embryos.

Any culture medium suitable for culturing embryos may be used. For example, Global® medium by LifeGlobal (www.lifeglobal.com; containing physiological salts, energy substrates, pH buffer (bicarbonate or HEPES), essential amino acids, non-essential amino acids, glutamine dipeptide, EDTA, phenol red and gentamicin), GM501 medium by Gynemed (www.gynemed.de; containing NaCl, KCl, KH₂PO₄, MgSO₄·7H₂O, NaHCO₃, CaCl₂.2H₂O, D(+)-glucose anhydrous, Na-Lactate (50% solution), Na-Pyruvate, EDTA, Na-alanyl-glutamine, water, non-essential and essential amino acids) and SSM™ medium by IrvineScientific (www.irvinesci.com) are commercially available embryo culture media for one media system and can be used to culture embryos from about Day 0 to about Day 5/6. For sequential media system, Cleavage K-SICM (Day 1 to Day 3) and Blastocyst K-SIBM (Day 3 to Day 5/6) by Cook Medical (www.cookmedical.com), Quinns Advantage®Cleavage (Day 1 to Day 3) and Quinns Advantage®Blastocyst (Day 3 to Day 5/6) by CooperSurgical (www.coopersurgical.com), FERTICULT™IVF Medium (Day 1 to Day 2) and FERTICULT™ G3 Medium (Day 3 to Day 4) by FertiPro (www.fertipro.com), IVC-TWO™ (Day 0 to Day 3) and IVC-THREE™ (Day 3 to Day 5) by InVitroCare (www.invitrocare.com), ECM® (Day 0 to Day 3) and MultiBlast® (Day 3 to Day 5) by Irvine Scientific (www.irvinesci.com), EmbryoAssist™ (Day 0 to Day 3) and BlastAssist™ (Day 3 to Day 5) by Origio (www.origio.com), ISM1 (Day 0 to Day 3) and ISM2 (Day 3 to Day 5) by Origio, G-1™PLUS (Day 1 to Day 3) and G-2™PLUS (Day 3 to Day 5) by Vitrolife (www.vitrolife.com), and IVF™ (Day 0 to Day 3) and CCM™ (Day 3 to Day 5) by Vitrolife are commercially available embryo culture media and can be used. The key components of both modern media systems (one media system and sequential media system) are shown in Table 1 (adapted from Gruber et al., J. Turk. Ger. Gynecol. Assoc., 2011, 12(2): 110-117).

TABLE 1
Key components of modern media for embryo culture.

| | One media system | Sequential media system | |
|---|---|---|---|
| Components | Gynemed GM501 ® | G-1 ™PLUS | G-2 ™PLUS |
| Salts | Sodium chloride | Sodium chloride | Sodium chloride |
| | Potassium chloride | Potassium chloride | Potassium chloride |
| | Calcium chloride | Calcium chloride | Calcium chloride |
| | Monopotassium | Sodium citrate | Sodium citrate |
| | phosphate | | |
| | Magnesium sulphate | Magnesium sulphate | Magnesium sulphate |
| | | Sodium dihydrogen | Sodium dihydrogen |
| | | phosphate | phosphate |
| Buffer | Sodium bicarbonat | Sodium bicarbonat | Sodium bicarbonat |
| Energy Substrates | Glucose | Glucose | Glucose |
| | Sodium lactate | Sodium lactate | Sodium lactate |
| | Sodium pyruvate | Sodium pyruvate | Sodium pyruvate |
| Non-Essential AA's | NEAA's 8 | NEAA's 9 | NEAA's |
| Glutamine Dipeptide | Alanyl-Glutamine | | |
| Essential AA's | EAA's 2 | EAA's 11 | EAA's |
| Chelator | EDTA | EDTA | none |
| Macromolecules | none | Hyaluronan, HSA | Hyaluronan, HSA |
| Fatty acid | none | Lipoic acid | none |
| Vitamins | none | None | 4 Vitamins |
| Indicator | Phenol Red optional | None | none |
| Antibiotic | Gentamicin | Gentamicin | Gentamicin |
| Water | Yes | Yes | Yes |

The volume of the sample used in the system of the disclosure may vary. In some embodiments, the volume of the sample is from about 1 μL to about 40 μL. In some embodiments, the volume of the sample is from about 10 μL to about 40 μL. In some embodiments, the volume of the sample is from about 15 μL to about 40 μL. In some embodiments, the volume of the sample is from about 20 μL to about 40 μL. In some embodiments, the volume of the sample is from about 5 μL to about 35 μL. In some embodiments, the volume of the sample is from about μL to about 30 μL. In some embodiments, the volume of the sample is from about 5 μL to about μL. In some embodiments, the volume of the sample is from about 5 μL to about 20 μL. In some embodiments, the volume of the sample is from about 5 μL to about 15 μL. In some embodiments, the volume of the sample is from about 5 μL to about 10 μL. In some embodiments, the volume of the sample is about 40 μL. In some embodiments, the volume of the sample is about μL. In some embodiments, the volume of the sample is about 30 μL. In some embodiments, the volume of the sample is about 25 μL. In some embodiments, the volume of the sample is about 20 μL. In some embodiments, the volume of the sample is about 15 μL. In some embodiments, the volume of the sample is about 10 μL. In some embodiments, the volume of the sample is about 9 μL. In some embodiments, the volume of the sample is about 8 μL. In some embodiments, the volume of the sample is about 7 μL. In some embodiments, the volume of the sample is about 6 μL. In some embodiments, the volume of the sample is about 5 μL. In some embodiments, the volume of the sample is about 4 μL. In some embodiments, the volume of the sample is about 3 μL. In some embodiments, the volume of the sample is about 2 μL. In some embodiments, the volume of the sample is about 1 μL.

The system of disclosure has superior sensitivity and is capable of detecting PIF that is present in a sample in trace amount. In some embodiments, the system has a sensitivity of detecting PIF at a concentration from about 0.5 pg/mL to about 20 ng/mL in a sample. In some embodiments, the system has a sensitivity of detecting PIF at a concentration from about 0.5 pg to about 500 ng of PIF in the sample. In some embodiments, the system has a sensitivity of detecting PIF at a concentration from about 0.5 pg to about 10 ng of PIF in the sample. In some embodiments, the system has a sensitivity of detecting PIF at a concentration from about 0.5 pg to about 5 ng of PIF in the sample. In some embodiments, the system has a sensitivity of detecting PIF at a concentration from about 0.5 pg/mL to about 1 ng/mL in a sample. In some embodiments, the system has a sensitivity of detecting PIF at a concentration from about 1 pg/mL to about 500 pg/mL. In some embodiments, the system has a sensitivity of detecting PIF at a concentration from about 1 pg/mL to about 400 pg/mL. In some embodiments, the system has a sensitivity of detecting PIF at a concentration from about 1 pg/mL to about 300 pg/mL. In some embodiments, the system has a sensitivity of detecting PIF at a concentration from about 1 pg/mL to about 200 pg/mL. In some embodiments, the system has a sensitivity of detecting PIF at a concentration from about 1 pg/mL to about 300 pg/mL. In some embodiments, the system has a sensitivity of detecting PIF at a concentration from about 1 pg/mL to about 100 pg/mL. In some embodiments, the system has a sensitivity of detecting PIF at a concentration from about 5 pg/mL to about 50 pg/mL. In some embodiments, the system has a sensitivity of detecting PIF at a concentration from about 1 pg/mL to about 1000 pg/mL. In some embodiments, the system has a sensitivity of detecting PIF at a concentration from about 20 pg/mL to about 30 pg/mL. In some embodiments, the system has a sensitivity of detecting PIF at a lower end of detection limit of concentration of about 1 pg/mL, or about 2, 3, 4, 5, 6, 7, 8, 9, or 10 pg/mL.

In some embodiments, the sensitivity of the system of the disclosure is about any of the sensitivities disclosed in the Examples or Figures section of the disclosure. In some embodiments, the sensitivity is equal to detection of PIF peptide at a level of about 0.125 μM within the sample. In some embodiments, the sensitivity is equal to detection of PIF peptide at a level of about 0.120 μM within the sample. In some embodiments, the sensitivity is equal to detection of PIF peptide at a level of about 0.110 μM within the sample. In some embodiments, the sensitivity is equal to detection of PIF peptide at a level of about 0.140 μM within the sample. In some embodiments, the sensitivity is equal to detection of PIF peptide at a level of about 0.130 μM within the sample. In some embodiments, the sensitivity is equal to detection of PIF peptide at a level of about 0.150 μM within the sample. In some embodiments, the sensitivity is equal to detection of PIF peptide at a level of about 0.225 μM within the sample. In some embodiments, the sensitivity is equal to detection of PIF peptide at a level of about 0.175 μM within the sample. In some embodiments, the sensitivity is equal to detection of PIF peptide at a level of about 0.125 μM within the sample. In some embodiments, the sensitivity is equal to detection of PIF peptide at a level of about 0.200 μM within the sample. In some embodiments, the sensitivity is equal to detection of PIF peptide at a level of about 0.090, 0.091, 0.92, 0.093, 0.094, 0.095 μM within the sample. In some embodiments, the sensitivity is equal to detection of PIF peptide at a level of from about 10, 11, 12, 13, 14, or 15 nM to about 100 nM within the sample.

Calculation of the amount or concentration of PIF can be perform by use of any of a variety of methods known in the art. For example, standards and controls with known concentrations of PIF can be prepared and used in conjunction with a sample to be tested.

The disclosure further relates to an array comprising any of the disclosed monoclonal antibodies immobilized onto a solid support at a first addressable location. In some embodiments, the array of the disclosure further comprises a substrate for detecting a pregnancy-related hormone or protein immobilized onto the solid support at a second addressable location. In some embodiments, the array of the disclosure further comprises substrates for detecting pregnancy-related hormones or proteins, each substrate being immobilized onto the solid support at a different addressable location. Several hormones are known to be related to pregnancy, such as human chorionic gonadotropin hormone (hCG), human placental lactogen (hPL, also known as human chorionic somatomammotropin (CSH1)), estrogen, and progesterone. HLA-G histocompatibility antigen, class I, G, also known as human leukocyte antigen G or HLA-G, is also known to be associated with successful pregnancy. In some embodiments, the array of the disclosure comprises any of the disclosed monoclonal antibodies immobilized onto a solid support at a first addressable location and a substrate for detecting hCG immobilized onto the solid support at a second addressable location. In some embodiments, the array of the disclosure comprises any of the disclosed monoclonal antibodies immobilized onto a solid support at a first addressable location and a substrate for detecting hPL immobilized onto the solid support at a second addressable location. In some embodiments, the array of the disclosure comprises any of the disclosed monoclonal antibodies immobilized onto a solid support at a first addressable location and a substrate for detecting estrogen immobilized onto the solid support at a second addressable location. In some embodiments, the array of the disclosure comprises any of the disclosed monoclonal antibodies immobilized onto a solid support at a first addressable location and a substrate for detecting progesterone immobilized onto the solid support at a second addressable location. In some embodiments, the array of the disclosure comprises any of the disclosed monoclonal antibodies immobilized onto a solid support at a first addressable location and a substrate for detecting HLA-G immobilized onto the solid support at a second addressable location. In some embodiments, the array of the disclosure comprises any of the disclosed monoclonal antibodies immobilized onto a solid support at a first addressable location, a substrate for detecting hCG immobilized onto the solid support at a second addressable location, and a substrate for detecting hPL immobilized onto the solid support at a third addressable location. In some embodiments, the array of the disclosure comprises any of the disclosed monoclonal antibodies immobilized onto a solid support at a first addressable location, a substrate for detecting hCG immobilized onto the solid support at a second addressable location, and a substrate for detecting estrogen immobilized onto the solid support at a third addressable location. In some embodiments, the array of the disclosure comprises any of the disclosed monoclonal antibodies immobilized onto a solid support at a first addressable location, a substrate for detecting hCG immobilized onto the solid support at a second addressable location, and a substrate for detecting progesterone immobilized onto the solid support at a third addressable location. In some embodiments, the array of the disclosure comprises any of the disclosed monoclonal antibodies immobilized onto a solid support at a first addressable location, a substrate for detecting hCG immobilized onto the solid support at a second addressable location, and a substrate for detecting HLA-G immobilized onto the solid support at a third addressable location. In some embodiments, the array of the disclosure comprises any of the disclosed monoclonal antibodies immobilized onto a solid support at a first addressable location, a substrate for detecting hPL immobilized onto the solid support at a second addressable location, and a substrate for detecting estrogen immobilized onto the solid support at a third addressable location. In some embodiments, the array of the disclosure comprises any of the disclosed monoclonal antibodies immobilized onto a solid support at a first addressable location, a substrate for detecting hPL immobilized onto the solid support at a second addressable location, and a substrate for detecting progesterone immobilized onto the solid support at a third addressable location. In some embodiments, the array of the disclosure comprises any of the disclosed monoclonal antibodies immobilized onto a solid support at a first addressable location, a substrate for detecting hPL immobilized onto the solid support at a second addressable location, and a substrate for detecting HLA-G immobilized onto the solid support at a third addressable location. In some embodiments, the array of the disclosure comprises any of the disclosed monoclonal antibodies immobilized onto a solid support at a first addressable location, a substrate for detecting estrogen immobilized onto the solid support at a second addressable location, and a substrate for detecting progesterone immobilized onto the solid support at a third addressable location. In some embodiments, the array of the disclosure comprises any of the disclosed monoclonal antibodies immobilized onto a solid support at a first addressable location, a substrate for detecting estrogen immobilized onto the solid support at a second addressable location, and a substrate for detecting HLA-G immobilized onto the solid support at a third addressable location. In some embodiments, the array of the disclosure comprises any of the disclosed monoclonal antibodies immobilized onto a solid support at a first addressable location, a substrate for detecting progesterone immobilized onto the solid support at a second addressable location, and a substrate for detecting HLA-G immobilized onto the solid support at a third addressable location. In some embodiments, the array of the disclosure comprises any of the disclosed monoclonal antibodies immobilized onto a solid support at a first addressable location, a substrate for detecting hCG immobilized onto the solid support at a second addressable location, a substrate for detecting hPL immobilized onto the solid support at a third addressable location, and a substrate for detecting estrogen immobilized onto the solid support at a fourth addressable location. In some embodiments, the array of the disclosure comprises any of the disclosed monoclonal antibodies immobilized onto a solid support at a first addressable location, a substrate for detecting hCG immobilized onto the solid support at a second addressable location, a substrate for detecting hPL immobilized onto the solid support at a third addressable location, and a substrate for detecting progesterone immobilized onto the solid support at a fourth addressable location. In some embodiments, the array of the disclosure comprises any of the disclosed monoclonal antibodies immobilized onto a solid support at a first addressable location, a substrate for detecting hCG immobilized onto the solid support at a second addressable location, a substrate for detecting hPL immobilized onto the solid support at a third addressable location, and a substrate for detecting HLA-G immobilized onto the solid support at a fourth addressable location. In some embodiments, the array of the disclosure comprises any of the disclosed monoclonal antibodies immobilized onto a solid support at a first addressable location, a substrate for detecting hCG immobilized onto the solid support at a second addressable location, a substrate for detecting estrogen immobilized onto the solid support at a third addressable location, and a substrate for detecting progesterone immobilized onto the solid support at a fourth addressable location. In some embodiments, the array of the disclosure comprises any of the disclosed monoclonal antibodies immobilized onto a solid support at a first addressable location, a substrate for detecting hCG immobilized onto the solid support at a second addressable location, a substrate for detecting estrogen immobilized onto the solid support at a third addressable location, and a substrate for detecting HLA-G immobilized onto the solid support at a fourth addressable location. In some embodiments, the array of the disclosure comprises any of the disclosed monoclonal antibodies immobilized onto a solid support at a first addressable location, a substrate for detecting hCG immobilized onto the solid support at a second addressable location, a substrate for detecting progesterone immobilized onto the solid support at a third addressable location, and a substrate for detecting HLA-G immobilized onto the solid support at a fourth addressable location. In some embodiments, the array of the disclosure comprises any of the disclosed monoclonal antibodies immobilized onto a solid support at a first addressable location, a substrate for detecting hPL immobilized onto the solid support at a second addressable location, a substrate for detecting estrogen immobilized onto the solid support at a third addressable location, and a substrate for detecting progesterone immobilized onto the solid support at a fourth addressable location. In some embodiments, the array of the disclosure comprises any of the disclosed monoclonal antibodies immobilized onto a solid support at a first addressable location, a substrate for detecting hPL immobilized onto the solid support at a second addressable location, a substrate for detecting estrogen immobilized onto the solid support at a third addressable location, and a substrate for detecting HLA-G immobilized onto the solid support at a fourth addressable location. In some embodiments, the array of the disclosure comprises any of the disclosed monoclonal antibodies immobilized onto a solid support at a first addressable location, a substrate for detecting hPL immobilized onto the solid support at a second addressable location, a substrate for detecting progesterone immobilized onto the solid support at a third addressable location, and a substrate for detecting HLA-G immobilized onto the solid support at a fourth addressable location. In some embodiments, the array of the disclosure comprises any of the disclosed monoclonal antibodies immobilized onto a solid support at a first addressable location, a substrate for detecting estrogen immobilized onto the solid support at a second addressable location, a substrate for detecting progesterone immobilized onto the solid support at a third addressable location, and a substrate for detecting HLA-G immobilized onto the solid support at a fourth addressable location.

In some embodiments, the array of the disclosure comprises any of the disclosed monoclonal antibodies immobilized onto a solid support at a first addressable location, a substrate for detecting hCG immobilized onto the solid support at a second addressable location, a substrate for detecting hPL immobilized onto the solid support at a third addressable location, a substrate for detecting estrogen immobilized onto the solid support at a fourth addressable location, and a substrate for detecting progesterone immobilized onto the solid support at a fifth addressable location. In some embodiments, the array of the disclosure comprises any of the disclosed monoclonal antibodies immobilized onto a solid support at a first addressable location, a substrate for detecting hCG immobilized onto the solid support at a second addressable location, a substrate for detecting hPL immobilized onto the solid support at a third addressable location, a substrate for detecting estrogen immobilized onto the solid support at a fourth addressable location, and a substrate for detecting HLA-G immobilized onto the solid support at a fifth addressable location. In some embodiments, the array of the disclosure comprises any of the disclosed monoclonal antibodies immobilized onto a solid support at a first addressable location, a substrate for detecting hCG immobilized onto the solid support at a second addressable location, a substrate for detecting hPL immobilized onto the solid support at a third addressable location, a substrate for detecting progesterone immobilized onto the solid support at a fourth addressable location, and a substrate for detecting HLA-G immobilized onto the solid support at a fifth addressable location. In some embodiments, the array of the disclosure comprises any of the disclosed monoclonal antibodies immobilized onto a solid support at a first addressable location, a substrate for detecting hCG immobilized onto the solid support at a second addressable location, a substrate for detecting estrogen immobilized onto the solid support at a third addressable location, a substrate for detecting progesterone immobilized onto the solid support at a fourth addressable location, and a substrate for detecting HLA-G immobilized onto the solid support at a fifth addressable location. In some embodiments, the array of the disclosure comprises any of the disclosed monoclonal antibodies immobilized onto a solid support at a first addressable location, a substrate for detecting hPL immobilized onto the solid support at a second addressable location, a substrate for detecting estrogen immobilized onto the solid support at a third addressable location, a substrate for detecting progesterone immobilized onto the solid support at a fourth addressable location, and a substrate for detecting HLA-G immobilized onto the solid support at a fifth addressable location. In some embodiments, the array of the disclosure comprises any of the disclosed monoclonal antibodies immobilized onto a solid support at a first addressable location, a substrate for detecting hCG immobilized onto the solid support at a second addressable location, a substrate for detecting hPL immobilized onto the solid support at a third addressable location, a substrate for detecting estrogen immobilized onto the solid support at a fourth addressable location, a substrate for detecting progesterone immobilized onto the solid support at a fifth addressable location, and a substrate for detecting HLA-G immobilized onto the solid support at a sixth addressable location.

Any substrates for detecting hCG, hPL, estrogen, progesterone and HLA-G can be used in the array of the disclosure. For example, commercially available antibodies for detecting hCG include, but not limited to, mouse monoclonal anti-hCG antibody SC1 (Invitrogen, Carlsbad, CA, Catalog #MA5-14680), mouse monoclonal anti-hCG antibody SC2 (Invitrogen, Carlsbad, CA, Catalog #MA5-14701), mouse monoclonal anti-hCG antibody 3002 (Invitrogen, Carlsbad, CA, Catalog #MA5-14699), mouse monoclonal anti-hCG antibody 3015 (Invitrogen, Carlsbad, CA, Catalog #MA5-14700), mouse monoclonal anti-hCG antibody 34P2C2 (Invitrogen, Carlsbad, CA, Catalog #MA5-14702), mouse monoclonal anti-hCG antibody P6F11G5 (Invitrogen, Carlsbad, CA, Catalog #MA5-14751), recombinant rabbit monoclonal anti-hCG antibody RM330 (Invitrogen, Carlsbad, CA, Catalog #MA5-33036), rabbit polyclonal anti-hCG beta antibody LS-B7043 (LifeSpan BioSciences, Seattle, WA, Catalog #LS-B7043-50), and sheep polyclonal anti-hCG beta antibody (Bio-Rad, Hercules CA, Catalog #2090-1536). Commercially available antibodies for detecting hPL include, but not limited to, mouse monoclonal anti-hPL antibody 658230 (Novus Biologicals, Littleton, CO, Catalog #MAB5757-SP), rabbit polyclonal anti-hPL antibody (Novus Biologicals, Littleton, CO, Catalog #35080002), rabbit polyclonal anti-hPL antibody LS-C405051 (LifeSpan BioSciences, Seattle, WA, Catalog #LS-C405051-20), mouse monoclonal anti-hPL antibody 658230 (R & D Systems, Minneapolis, MN, Catalog #MAB5757), mouse monoclonal anti-hPL (CSH1) antibody OTI1C9 (Invitrogen, Carlsbad, CA, Catalog #MA5-26275), mouse monoclonal anti-hPL (CSH1) antibody OTI4D6 (Invitrogen, Carlsbad, CA, Catalog #MA5-26276), and mouse monoclonal anti-hPL (CSH1) antibody INN-hPL-37 (Invitrogen, Carlsbad, CA, Catalog #MA1-25113). Commercially available antibodies for detecting estrogen include, but not limited to, mouse monoclonal anti-estradiol antibody AB4.2 (Invitrogen, Carlsbad, CA, Catalog #MA5-14748), mouse monoclonal anti-estradiol antibody 6E1 (Invitrogen, Carlsbad, CA, Catalog #MA1-83784), mouse monoclonal anti-estradiol antibody AB5.1 (Invitrogen, Carlsbad, CA, Catalog #MIE0108), recombinant rabbit monoclonal anti-estradiol antibody RM343 (Invitrogen, Carlsbad, CA, Catalog #MA5-27910), and polyclonal rabbit estrogen antibody LS-C128893 (LifeSpan BioSciences, Seattle, WA, Catalog #LS-C128893). Commercially available antibodies for detecting progesterone include, but not limited to, mouse monoclonal anti-progesterone antibody 1188/344 (Bio-Rad, Hercules CA, Catalog #7720-0000), mouse monoclonal anti-progesterone antibody 9B4 (Bio-Rad, Hercules CA, Catalog #7720-1704), mouse monoclonal anti-progesterone antibody BGN/6-5E-10B (Bio-Rad, Hercules CA, Catalog #7720-1430), and mouse monoclonal anti-progesterone antibody 45/23 (Invitrogen, Carlsbad, CA, Catalog #MA5-18037). Commercially available antibodies for detecting HLA-G include, but not limited to, mouse monoclonal anti-HLA-G antibody MEM-G/1 (Invitrogen, Carlsbad, CA, Catalog #MA1-19219), mouse monoclonal anti-HLA-G antibody 87G (Invitrogen, Carlsbad, CA, Catalog #MA1-10356), mouse monoclonal anti-HLA-G antibody 1E5A10 (manufactured by Proteintech and available at Invitrogen, Catalog #66447-1-IG), and mouse monoclonal anti-HLA-G antibody 1D6 (manufactured by Abnova and available at Invitrogen, Catalog #H00003135-M01).

Methods

The disclosure further relates to methods of using any of the disclosed monoclonal antibodies or any of the disclosed systems. In some embodiments, the disclosure relates to a method of detecting embryo fitness for implantation comprising detecting the presence, absence, and/or biologically significant quantity of a PIF peptide, or an immunogenic fragment or epitope thereof, in a sample using any of the disclosed monoclonal antibodies or any of the disclosed systems. In some embodiments, the presence of the PIF detected in the sample is indicative of embryo fitness for implantation. In some embodiments, the disclosure further relates to a method of detecting a PIF in an embryonic culture medium comprising detecting the presence, absence, and/or biologically significant quantity of a PIF peptide, or an immunogenic fragment or epitope thereof, in a sample using any of the disclosed monoclonal antibodies or any of the disclosed systems. In some embodiments, presence of the PIF in the sample is indicative of the presence of one or a plurality of viable embryos in the embryo culture medium from which the sample is obtained.

The sample used in any of the above disclosed methods can be obtained from a spent embryo culture medium used for culturing one or a plurality of embryos. In some embodiments, the sample is a sample from culture medium exposed to a single human embryo in culture. I some embodiments, the sample was exposed to the single embryo for more than one day. I some embodiments, the sample is an embryo culture medium from Table 1 comprising PIF or a variant thereof that is measured against a control sample of embryo culture medium from Table 1 not exposed to an embryo in culture. Human embryos begin to express PIF at the 2-cell stage. In some embodiments therefore, the sample is obtained from a single-embryo culture that is at a two-cell stage. In some embodiments, the sample is obtained from a multi-embryo culture medium.

In some embodiments, the sample is obtained from an embryo culture medium used for culturing one or a plurality of embryos at about Day 1 after culturing the one or plurality of embryos. In some embodiments, the sample is obtained from an embryo culture medium used for culturing one or a plurality of embryos at about Day 2 after culturing the one or plurality of embryos. In some embodiments, the sample is obtained from an embryo culture medium used for culturing one or a plurality of embryos at about Day 3 after culturing the one or plurality of embryos. In some embodiments, the sample is obtained from an embryo culture medium used for culturing one or a plurality of embryos at about Day 4 after culturing the one or plurality of embryos. In some embodiments, the sample is obtained from an embryo culture medium used for culturing one or a plurality of embryos at about Day 5 after culturing the one or plurality of embryos. In some embodiments, the sample is obtained from an embryo culture medium used for culturing one or a plurality of embryos at about Day 6 after culturing the one or plurality of embryos. In some embodiments, the sample is obtained from an embryo culture medium used for culturing one or a plurality of embryos at about Day 7 after culturing the one or plurality of embryos. In some embodiments, the sample is obtained from an embryo culture medium used for culturing one or a plurality of embryos at about Day 8 after culturing the one or plurality of embryos. In some embodiments, the sample is obtained from an embryo culture medium used for culturing one or a plurality of embryos at about Day 9 after culturing the one or plurality of embryos. In some embodiments, the sample is obtained from an embryo culture medium used for culturing one or a plurality of embryos at about Day 10 after culturing the one or plurality of embryos.

In some embodiments, the sample is obtained from an embryo culture medium used for culturing one or a plurality of embryos at about Day 3 to about Day 10 after culturing the one or plurality of embryos. In some embodiments, the sample is obtained from an embryo culture medium used for culturing one or a plurality of embryos at about Day 3 to about Day 7 after culturing the one or plurality of embryos. In some embodiments, the sample is obtained from an embryo culture medium used for culturing one or a plurality of embryos at about Day to about Day 6 after culturing the one or plurality of embryos. In some embodiments, the sample is obtained from an embryo culture medium used for culturing one or a plurality of embryos at about Day 3 to about Day 5 after culturing the one or plurality of embryos. In some embodiments, the sample is obtained from an embryo culture medium used for culturing one or a plurality of embryos at about Day 3 to about Day 6 after culturing the one or plurality of embryos. In some embodiments, the sample is obtained from an embryo culture medium used for culturing one or a plurality of embryos at about Day 4 to about Day 6 after culturing the one or plurality of embryos.

The volume of the sample taken from the embryo culture medium may vary. In some embodiments, the volume of the sample is from about 1 μL to about 40 μL. In some embodiments, the volume of the sample is from about 2 μL to about 35 μL. In some embodiments, the volume of the sample is from about 3 μL to about 30 μL. In some embodiments, the volume of the sample is from about 4 μL to about 25 μL. In some embodiments, the volume of the sample is from about μL to about 20 μL. In some embodiments, the volume of the sample is from about 6 μL to about μL. In some embodiments, the volume of the sample is from about 7 μL to about 10 μL. In some embodiments, the volume of the sample is about 40 μL. In some embodiments, the volume of the sample is about 35 μL. In some embodiments, the volume of the sample is about 30 μL. In some embodiments, the volume of the sample is about 25 μL. In some embodiments, the volume of the sample is about 20 μL. In some embodiments, the volume of the sample is about 15 μL. In some embodiments, the volume of the sample is about 10 μL. In some embodiments, the volume of the sample is about 9 μL. In some embodiments, the volume of the sample is about 8 μL. In some embodiments, the volume of the sample is about 7 μL. In some embodiments, the volume of the sample is about 6 μL. In some embodiments, the volume of the sample is about 5 μL. In some embodiments, the volume of the sample is about 4 μL. In some embodiments, the volume of the sample is about 3 μL. In some embodiments, the volume of the sample is about 2 μL. In some embodiments, the volume of the sample is about 1 μL.

In some embodiments, the sample used in any of the methods disclosed herein may be further diluted for assaying. In some embodiments, dilutions are made with the same culture medium that is used for culturing embryos. Examples of such culture medium include, but not limited to, G-1′ Plus and G-2′ Plus Media manufactured by Vitrolife (Sweden), EmbryoMax® KSOM Mouse Embryo Media manufactured by Sigma-Aldrich (St. Louis, MO), InVitroCare™ IVC-THREE™ Blastocyst Medium manufactured by InVitroCare Inc. (Frederick, MD), and any culture media provided elsewhere herein. In some embodiments, dilutions are made with assaying buffer that is use for preparing the standard and control. Examples of such assaying buffer include, but not limited to B-PBST (PBS+0.5% Tween 20 (PBST) supplemented with 1% BSA).

In some embodiments, the sample is used in the disclosed method at a dilution ratio of 5:1. In some embodiments, the sample is used in the disclosed method at a dilution ratio of 10:1. In some embodiments, the sample is used in the disclosed method at a dilution ratio of 20:1. In some embodiments, the sample is used in the disclosed method at a dilution ratio of 30:1. In some embodiments, the sample is used in the disclosed method at a dilution ratio of 40:1. In some embodiments, the sample is used in the disclosed method at a dilution ratio of 50:1. In some embodiments, the sample is used in the disclosed method at a dilution ratio of 60:1. In some embodiments, the sample is used in the disclosed method at a dilution ratio of 70:1. In some embodiments, the sample is used in the disclosed method at a dilution ratio of 80:1. In some embodiments, the sample is used in the disclosed method at a dilution ratio of 90:1. In some embodiments, the sample is used in the disclosed method at a dilution ratio of 100:1.

A person skilled in the art would readily recognize that any method for detecting the presence, absence and/or quantity of a protein in a sample may be used to practice any of the methods disclosed herein to determine the presence, absence and/or quantity of a PIF in a sample. For instance, the presence, absence and/or quantity of PIF in a sample may be detected by techniques that employ the principle of internal reflection of polarized light, such as surface plasmon resonance (SPR), by immunoassays, such as enzyme-linked immunosorbent assay (ELISA), by microarray, and by trap-and-elute LC-MS/MS methods. The presence, absence and/or quantity of PIF in a sample may be detected by traditional structural biology methods such as X-ray crystallography, mass spectrometry, NMR spectroscopy, and electron microscopy (EM),

In some embodiments, the presence, absence and/or quantity of PIF in a sample is detected by techniques that employ the principle of internal reflection of polarized light. Such detection methods may be based on the evanescent wave phenomenon at total internal reflection, such as surface plasmon resonance (SPR), critical angle refractometry, total internal reflection fluorescence (TIRF), total internal reflection phosphorescence, total internal reflection light scattering, and evanescent wave ellipsometry. The detection method may also be based on Brewster angle reflectometry. In some embodiments, the presence, absence and/or quantity of PIF in a sample is detected by SPR. SPR, in somewhat simplified terms, may be said to be a technique according to which changes in the refractive index of a layer close to a thin free-electron metal film are detected by way of consequential changes in the intensity of a p-polarized light beam reflected from the metal film (see for example, Raether H, Physics of Thin Films, Academic Press, N.Y., 9 (1977) 145).

In the first publication indicating the possibilities of SPR technology in biochemical analysis, Liedberg B et al. (Sensors and Actuators, 4 (1983) 299) have at first adsorbed a monolayer of IgG to a silver surface and then adsorbed to said monolayer a layer of anti-IgG, in order to then study the effect in respect of the resultant change in the resonance angle. EP 202 021 describes a biosensor employing movable optical instrumentation for determining the angle—henceforth called resonance angle—at which surface plasmon resonance occurs. Such movable optical units are not suitable for commercial-type instruments because (i) when readings of the resonance angle are to be taken this will require manual operations, and (ii) technical manufacturing tolerances in the suspension mechanism of the movable optical system are contributory to errors occurring in the measurements of the resonance angle. EP 257 955 describes another optical system which is scanned mechanically for determining the resonance angle. GB 2 197 068 describes an optical sensor employing a divergent beam of rays for irradiating the sensitized surface, this latter being a metal film with receptors or ligands which interact selectively with one or more bio-molecules. The optical system is stationary, so the above-mentioned drawbacks of movable optical systems are avoided. A source of light is employed for irradiating a sensitized surface which is subjected to the action of a sample solution while another source of light is employed for irradiating another sensitized surface which is subjected to the action of a reference solution. The light sources and sensitized surfaces are arranged in such a way that the reflected divergent beams will strike a photodetector matrix. By means of alternate activations of each one of the two light sources the resonance angle obtained from each of the two sensitized surfaces can be measured with a good degree of precision, and the difference between the two resonance angles at each of the two sensitized surfaces will be a measure of the amount of the specific biomolecule bound on the sensitive layer. The disadvantage of this apparatus resides in the use of two individual sources of light—one for the reference solution, one for the sample solution—as this will tend to make the measuring result uncertain in view of the fact that the resonance angle is highly dependent on the spectral character of the light source. Another drawback of this known optical sensor resides in the positioning thereof directly on the prism of the optical system, and in having light directed to the sensitized surface via an immersion oil that has a suitable refractive index. Such a use of light-coupling oils will involve much practical inconvenience when the sensor unit, comprising a sensitized metal layer coated on a transparent plate, is to be replaced by a new sensor unit with a sensing surface having an affinity for a different specific biomolecule. The replacement operations will inevitably give rise to oil smears, and the prism has to be cleaned before a new sensing surface can be analyzed. Manipulation of the instrument will thus be a somewhat messy business. As to the actual structure of the analytical instrument, this is not disclosed in the aforesaid GB patent specification. EP 226 470 describes an apparatus for microchemical analyses comprising two glass plates with a gel placed between them. The apparatus is one of the disposable type, to be used only once. One of the two glass plates serves as a platform on which the sample liquid is applied. Capillary force will then draw the sample liquid into the capillary cell that has been formed between the plates. A device of this type, the dimensions of which are about 3×1.5 cm, requires the use of tweezers or the like for handling. It is difficult to determine the volume of the liquid sample, and this device is therefore unsuitable for quantitative analyses.

EP 0 305 109 describes a SPR sensor system employing a focused (fan-shaped) light beam to illuminate the sensitive surface through a curved transparent block and via an index matching fluid. The beam enters the transparent block in a direction orthogonal to the tangent of the surface of the transparent block.

As was published by Kretschmann, E. (Optics Communications, 26, (1978) 41-44), the problem of slow speed of operation relative to changes in reflectance and the insufficient precision in the resonance angle determination related with SPR procedures based on moveable mechanics, is solved by the use of a fan-shaped beam (equivalent to several beams incident upon the sensor surface over a range of angles) and of collection of the reflected beams (over a range of angles) by an array of angularly spaced detectors.

Furthermore, the transparent block described in EP 0305 109 may take the form of a hemicylinder creating a wedge-shaped beam, giving a line of a small illuminated area on the sensing surface. The hemicylindrical lens has the advantage that it can be used to perform several tests simultaneously on a single sample. To this end, the sensing surface takes the form of a series of sensitive areas, each comprising a different antibody, with each separate area being monitored by its own detector in a detector array. The cylindrical focusing principle used to produce an identical angular range of light beams along a focused line for SPR of separate surface areas has been published by Benner, R. E. et al. (Optics Communications 30 (1979) 145-149) and Swalen, J D et al. (Am. J. Phys. 48 (1980) 669-672).

Further, a focusing lens in EP 0305 109 creates a substantially parallel-sided beam incident upon the detector, or a beam of at least of reduced divergence compared to the fan-shaped spread of light reflected from the sensing surface with the object to reduce stray light reflections in the detector array. The disadvantages of this apparatus resides primarily in the following. The approach to use a small illuminated area in relation to the sensitive layer for sensing, in order to reduce effects due to inevitable variations in a commercially produced metal film and coating of antibody. In fact, the surface concentration of bound sample molecules will in general also be non-uniform across the sensitive layer and strongly dependent on mass transport conditions. Thus, the small sensing area will be very sensitive to local variations of the sensing surface and its sample surface concentration resulting in a non-competitively low accuracy in the SPR response. Due to stray light arising from the coupling optics and at reflection in the sensor surface, it is possible to use the described optical system for monitoring beams from separate sensitive areas simultaneously by its own detector in an array only under the condition that the array can be conveniently placed close to the exit surface of the hemicylinder or attached to or deposited on that surface. This leads to limitations in the resolution of individual sensitive areas on the detector array, expensive optoelectronic constructions, complicated production process (detector alignment, optimization of collected angle span etc.). The use of an optical oil or grease to ensure good optical coupling between the hemisphere and the sensor substrate (glass support plate or slide).

In some embodiments, the presence, absence and/or quantity of PIF in a sample is detected by an immunoassay. The disclosure contemplates any form of immunoassay, including Western blotting, enzyme-linked immunosorbent assay (ELISA), fluorescence-linked immunosorbent assay (FLISA), competition assay, radioimmunoassay, lateral flow immunoassay, flow-through immunoassay, electrochemiluminescent assay, nephelometric-based assays, turbidometric-based assay, and fluorescence activated cell sorting (FACS)-based assays. In some embodiments, the presence, absence and/or quantity of PIF in a sample is detected by SPR immunoassay. In some embodiments, the presence, absence and/or quantity of PIF in a sample is detected by using an ELISA, such as a colorimetric ELISA or chemiluminescent ELISA. In some embodiments, the ELISA is a colorimetric ELISA. In some embodiments, the ELISA is a chemiluminescent ELISA.

ELISA is an analytical biochemistry assay that uses a solid-phase enzyme immunoassay (EIA) to detect the presence of a ligand (commonly a protein) in a liquid sample using antibodies directed against the protein to be measured. Various types of ELISA have been developed and routinely used in a wide range of applications, which can all be used to practice any of the methods disclosed herein. In some embodiments, the ELISA is a comparative ELISA in which a labelled synthetic PIF is used as a “tracer” to compete with a non-labeled PIF from the sample or standard using a constant and limiting amount of anti-PIF antibody. A decreasing amount of tracer binds to the antibody as the amount of PIF in the sample or standard is increased. Separation of the antibody-PIF complexes from free PIF is achieved by capture of the anti-PIF-tracer complex using a secondary antibody coated to the micro plate. The level of PIF in the sample is then determined using a standard curve that has been produced using known quantities of PIF or by comparison to a control sample. Examples of such assays are illustrated in, for instance, Examples 3 and 4 below.

The synthetic PIF, or tracer, may be labelled with a fluorescently or chemiluminescently detectable reporter molecule by use of any of a variety of methods. In some embodiments, the synthetic PIF is labelled fluorescently. In some embodiments, the synthetic PIF is labelled chemiluminescently. The fluorescent or chemiluminescent label can be located at the N-terminus and/or C-terminus of the synthetic PIF. Examples of such a detectable reporter molecule include, but not limiting to, an enzyme (e.g. horseradish peroxidase (HRP), alkaline phosphatase (AP), or 3-galactosidase) in the case of an ELISA or a fluorophore in the case of a FLISA. Following washing to remove any unbound protein, the detectable reporter molecule can be detected by the addition of a substrate in the case of an ELISA, such as for example, hydrogen peroxide, TMB, toluidine, or 5-bromo-4-chloro-3-indol-beta-D-galactopyranoside (x-gal). In some embodiments, the synthetic PIF is labelled with HRP at the N-terminus of the PIF to form a PIF-HRP conjugate.

In some embodiments, the disclosed method further comprises exposing the sample to one or a plurality of substrates, or tracers, capable of binding to the monoclonal antibody. In some embodiments, the substrate is fluorogenic, fluorescent, or chemiluminescent fluorescently labeled with one or a plurality of the fluorescent dye molecules disclosed elsewhere in the disclosure. In some embodiments, the substrate is a synthetic PIF peptide, or an immunogenic fragment or epitope thereof. In some embodiments, the substrate is a synthetic PIF peptide comprising at least about 70%, 75%, 80%, 85%, 90%, 95%, 96%, 97%, 98% or 99% sequence identity to SEQ ID NO: 11, or a functional fragment thereof. In some embodiments, the substrate is a PIF peptide, a functional fragment thereof, or an immunogenic fragment or epitope thereof, that is fluorescently labeled with one or a plurality of the aforementioned fluorescent dye molecules. In some embodiments, the fluorescent dye molecule is covalently or non-covalently bound to the PIF peptide at the N-terminus. In some embodiments, the fluorescent dye molecule is covalently or non-covalently bound to the PIF peptide at the C-terminus.

In some embodiments, the antibody-PIF complexes is separated from free PIF by capture of the antibody-PIF complexes using a secondary antibody against the species used for production of the monoclonal antibody. In some embodiments, the monoclonal antibody used in the methods of the disclosure is a rabbit monoclonal antibody and the secondary antibody used for capturing the antibody-PIF complexes formed in the assay is a mammalian anti-rabbit antibody. In some embodiments, the monoclonal antibody used in the methods of the disclosure is a rabbit monoclonal antibody and the secondary antibody is a goat anti-rabbit antibody. In some embodiments, the monoclonal antibody used in the methods of the disclosure is a rabbit monoclonal antibody and the secondary antibody is a sheep anti-rabbit antibody. In some embodiments, the monoclonal antibody used in the methods of the disclosure is a mouse monoclonal antibody and the secondary antibody used for capturing the antibody-PIF complexes formed in the assay is a mammalian anti-mouse antibody. In some embodiments, the monoclonal antibody used in the methods of the disclosure is a mouse monoclonal antibody and the secondary antibody is a rabbit anti-mouse antibody. In some embodiments, the monoclonal antibody used in the methods of the disclosure is a mouse monoclonal antibody and the secondary antibody is a goat anti-mouse antibody. In some embodiments, the monoclonal antibody used in the methods of the disclosure is a mouse monoclonal antibody and the secondary antibody is a sheep anti-mouse antibody. A skilled person would readily recognize that, when the animal species used for generating the monoclonal antibody of the disclosure changes, the source for the secondary antibody should change accordingly.

In some embodiments, the method of the disclosure comprises exposing a mixture of labeled- and non-labeled PIF to any of the disclosed monoclonal antibodies under conditions and for a sufficient time period to allow binding between the PIF (labeled or non-labeled) and the monoclonal antibody, and separating the non-binding PIF (labeled or non-labeled) from the PIF-antibody complexes thus formed. In some embodiments, the non-labeled PIF used in the disclosed methods is synthetic PIF with known concentration or quantity used as positive control. In some embodiments, the non-labeled PIF used in the disclosed methods is PIF contained in a spent embryo culture medium produced by one or a plurality of embryos cultured therein. Any methods known in the art for removing non-binding protein from an assay may be used. In some embodiments, a secondary antibody immobilized onto a solid support may be used to capture the PIF-antibody complexes and facilitate the separation of the PIF-antibody complexes from the non-binding PIF.

In some embodiments, the methods of the disclosure detects the amount of PIF in a sample by calculating the amount of intensity or presence of fluorescent in the PIF-antibody complexes caused by the labeled PIF. The amount of intensity or presence of fluorescent in a sample can be detected by any method known in the art. For instance, when the assay is performed on a microchip or biochip, the intensity or presence of fluorescent can be detected by a microarray scanner, such as GenePix Series Microarray Scanners (Molecular Devices), InnoScan Series Microarray Scanners (Innopsys), or SureScan Dx Series Microarray Scanners (Agilent Technologies). Laser scanners can also be used, the choice of which depends on the choice of dye used for detection.

In some embodiments, the methods of the disclosure further comprise a step of measuring the quantity of PIF in the sample by normalizing the quantity of the PIF detected in the sample with a measurement taken from a control sample. A “control sample” as used herein refers to a reference sample of known identity and known quantity. In some embodiments, the control sample is a negative control sample which does not contain PIF or fragments thereof. Negative control samples may include, but not limited to, water, assay buffer, or culture medium. In some embodiments, the negative control sample used in the methods of the disclosure is the embryo culture medium. In some embodiments, the control sample is a positive control sample which contains a known concentration or quantity of PIF. In some embodiments, more than one positive control samples, each having a different concentration or quantity of PIF, can be used, and thus, a standard curve can be generated for normalization purposes and calculating the concentration or quantity of PIF in the sample.

The disclosure relates to the detection of PIF in a sample, the method comprising: (i) obtaining a sample; and (ii) detecting whether PIF is present at biologically significant levels within the sample by contacting the sample with any of the disclosed monoclonal antibodies in the presence of a labeled PIF and detecting binding between the monoclonal antibody and the labeled PIF. In some embodiments, the biologically significant levels of PIF, or functional fragments thereof, within a sample are at or greater than about a 1 fold change in quantity as compared to the amount of PIF, or functional fragments thereof, in a control sample (for instance, a sample known to contain a predetermined amount of PIF). In some embodiments, the biologically significant levels of PIF, or functional fragments thereof, within a sample are at or greater than about a 1.1 fold change in quantity as compared to the amount of PIF, or functional fragments thereof, in a control sample (for instance, a sample known to contain a predetermined amount of PIF). In some embodiments, the biologically significant levels of PIF, or functional fragments thereof, within a sample are at or greater than about a 1.2 fold change in quantity as compared to the amount of PIF, or functional fragments thereof, in a control sample (for instance, a sample known to contain a predetermined amount of PIF). In some embodiments, the biologically significant levels of PIF, or functional fragments thereof, within a sample are at or greater than about a 1.3 fold change in quantity as compared to the amount of PIF, or functional fragments thereof, in a control sample (for instance, a sample known to contain a predetermined amount of PIF). In some embodiments, the biologically significant levels of PIF, or functional fragments thereof, within a sample are at or greater than about a 1.4 fold change in quantity as compared to the amount of PIF, or functional fragments thereof, in a control sample (for instance, a sample known to contain a predetermined amount of PIF). In some embodiments, the biologically significant levels of PIF, or functional fragments thereof, within a sample are at or greater than about a 1.5 fold change in quantity as compared to the amount of PIF, or functional fragments thereof, in a control sample (for instance, a sample known to contain a predetermined amount of PIF).

In some embodiments, any of the methods disclosed herein comprise a step of detecting the presence or biologically significant quantity of PIF in a sample with a sensitivity that is about any of the sensitivities disclosed in the Examples or Figures section of the disclosure. In some embodiments, the sensitivity of the disclosed methods is equal to detection of PIF peptide at a level of about 0.125 μM within the sample. In some embodiments, the sensitivity is equal to detection of PIF peptide at a level of about 0.120 μM within the sample. In some embodiments, the sensitivity is equal to detection of PIF peptide at a level of about 0.110 μM within the sample. In some embodiments, the sensitivity is equal to detection of PIF peptide at a level of about 0.140 μM within the sample. In some embodiments, the sensitivity is equal to detection of PIF peptide at a level of about 0.130 μM within the sample. In some embodiments, the sensitivity is equal to detection of PIF peptide at a level of about 0.150 μM within the sample. In some embodiments, the sensitivity is equal to detection of PIF peptide at a level of about 0.225 μM within the sample. In some embodiments, the sensitivity is equal to detection of PIF peptide at a level of about 0.175 μM within the sample. In some embodiments, the sensitivity is equal to detection of PIF peptide at a level of about 0.125 μM within the sample. In some embodiments, the sensitivity is equal to detection of PIF peptide at a level of about 0.200 μM within the sample. In some embodiments, the sensitivity is equal to detection of PIF peptide at a level of about 0.090, 0.091, 0.92, 0.093, 0.095 μM within the sample. In some embodiments, the sensitivity is equal to detection of PIF peptide at a level of from about 10, 11, 12, 13, 14, or 15 nM to about 100 nM within the sample.

In some embodiments, the disclosed method has a sensitivity of detecting PIF at a concentration from about 0.1 pg/mL to about 1 ng/mL in a sample. In some embodiments, the disclosed method has a sensitivity of detecting PIF at a concentration from about 1 pg/mL to about 500 pg/mL. In some embodiments, the disclosed method has a sensitivity of detecting PIF at a concentration from about 1 pg/mL to about 400 pg/mL. In some embodiments, the disclosed method has a sensitivity of detecting PIF at a concentration from about 1 pg/mL to about 300 pg/mL. In some embodiments, the disclosed method has a sensitivity of detecting PIF at a concentration from about 1 pg/mL to about 200 pg/mL. In some embodiments, the disclosed method has a sensitivity of detecting PIF at a concentration from about 1 pg/mL to about 100 pg/mL. In some embodiments, the disclosed method has a sensitivity of detecting PIF at a concentration from about 5 pg/mL to about 75 pg/mL. In some embodiments, the disclosed method has a sensitivity of detecting PIF at a concentration from about 5 pg/mL to about 50 pg/mL. In some embodiments, the disclosed method has a sensitivity of detecting PIF at a concentration from about 10 pg/mL to about 30 pg/mL. In some embodiments, the disclosed method has a sensitivity of detecting PIF at a concentration from about 20 pg/mL to about 30 pg/mL.

In some embodiments, the disclosed method has a sensitivity to detect at least about 20 ng of PIF in the sample. In some embodiments, the disclosed method has a sensitivity to detect at least about 15 ng of PIF in the sample. In some embodiments, the disclosed method has a sensitivity to detect at least about 10 ng of PIF in the sample. In some embodiments, the disclosed method has a sensitivity to detect at least about 5 ng of PIF in the sample. In some embodiments, the disclosed method has a sensitivity to detect at least about 1 ng of PIF in the sample. In some embodiments, the disclosed method has a sensitivity to detect at least about 500 pg of PIF in the sample. In some embodiments, the disclosed method has a sensitivity to detect at least about 250 pg of PIF in the sample. In some embodiments, the disclosed method has a sensitivity to detect at least about 100 pg of PIF in the sample. In some embodiments, the disclosed method has a sensitivity to detect at least about 50 pg of PIF in the sample. In some embodiments, the disclosed method has a sensitivity to detect at least about 25 pg of PIF in the sample. In some embodiments, the disclosed method has a sensitivity to detect at least about 10 pg of PIF in the sample. In some embodiments, the disclosed method has a sensitivity to detect at least about 1 pg of PIF in the sample. In some embodiments, the disclosed method has a sensitivity to detect at least about 0.5 pg of PIF in the sample. In some embodiments, the disclosed method has a sensitivity to detect at least about 0.1 pg of PIF in the sample. In some embodiments, the disclosed method has a sensitivity to detect at least about 0.05 pg of PIF in the sample. In some embodiments, the disclosed method has a sensitivity to detect at least about 0.01 pg of PIF in the sample.

In some embodiments, the disclosed method has a sensitivity to detect from about 0.01 pg to about 20 ng of PIF in the sample. In some embodiments, the disclosed method has a sensitivity to detect from about 0.05 pg to about 15 ng of PIF in the sample. In some embodiments, the disclosed method has a sensitivity to detect from about 0.1 pg to about 10 ng of PIF in the sample. In some embodiments, the disclosed method has a sensitivity to detect from about 0.5 pg to about 5 ng of PIF in the sample. In some embodiments, the disclosed method has a sensitivity to detect from about 1 μg to about 1 ng of PIF in the sample. In some embodiments, the disclosed method has a sensitivity to detect from about 0.01 pg to about 10 pg of PIF in the sample.

In some embodiments, the method may also include a step of correlating the quantity of PIF in the sample to the probability or likelihood that the culture from which the sample is obtained contains one or a plurality of viable embryos suitable for implantation by comparing to a measurement of the quantity of PIF in a control sample.

A skilled in the art would appreciate that such methods can be used for selecting a viable embryo for transplantation in an in vitro fertilization treatment of a subject. Thus, in some embodiments, the present disclosure also relates to a method of in vitro fertilization of a subject comprising: a) detecting the presence, absence, and/or biologically significant quantity of a PIF in an embryo culture medium using any of the disclosed monoclonal antibodies, or antigen-binding fragments thereof, or any of the disclosed systems; and b) transplanting at least one embryo into the subject, wherein the at least one embryo used for transplantation is obtained from a PIF-positive embryo culture. In some embodiments, only one embryo is transplanted. In some embodiments, two embryos from the same culture are transplanted. In some embodiments, two embryos from two different PIF-positive embryo cultures are transplanted. In some embodiments, more than two embryos are transplanted. In some embodiments, the subject is an animal, such as mammal or primate. In some embodiments, the subject is a human, such as a human undergoing treatment for infertility. In some embodiments, the subject is a human of age between 25 and 50 years old. In some embodiments, the subject is a human of age under 35 years old.

The disclosure further relates to a method of detecting a protein that binds to a PIF comprising: exposing the PIF, or an immunogenic fragment or epitope thereof, with any of the disclosed monoclonal antibodies in the presence of a test protein under conditions wherein, in the absence of the test protein, a pre-determined quantity of the PIF would bind the monoclonal antibody, and determining if the quantity of the PIF bound to the monoclonal antibody is decreased in the presence of the test protein, the decrease indicating that the test protein binds to the PIF. In some embodiments, the PIF, or the immunogenic fragment or epitope thereof, used in the method is fluorogenic, fluorescent, or chemiluminescent fluorescently labeled. Any of the fluorescent dye molecules disclosed elsewhere in the disclosure can be used to label the PIF, or the immunogenic fragment or epitope thereof. In some embodiments, the PIF used in the method comprises at least about 70%, 75%, 80%, 85%, 90%, 95%, 96%, 97%, 98% or 99% sequence identity to SEQ ID NO: 11. In some embodiments, the PIF used in the method comprises SEQ ID NO: 11. In some embodiments, the monoclonal antibody used in the method is a rabbit monoclonal antibody. In some embodiments, the concept of using a secondary antibody to facilitate the detection of binding disclosed elsewhere in the disclosure can be used.

Other embodiments are described in the following non-limiting Examples. Various publications, including patents, published applications, technical articles and scholarly articles are cited throughout the specification. Each of these cited publications is incorporated by reference herein in its entirety

### EXAMPLES

### Example 1: Rabbit Monoclonal Anti-PIF Antibodies

Rabbit monoclonal antibodies against human PIF peptide were generated using the Single Plasma Cell Interrogation (SPIN®) platform by ExonBio (San Diego, CA). Among numerous clones isolated, about 5 were identified as good performers. One of these good performers, clone #30, was later determined to be the best performer and sequenced. The sequences of the heavy chain and light chain of this clone are as follows.

Heavy chain (Clone #30)-nucleic acid
atggagactgggctgcgctggcttctcctggtcgctgtgctcaaaggtgtccagtgtcggtcgctggaggagtccgg
gggtcgcctggtcacgcctgggacacccctgacactcacctgcacagtctctggaatcgacctcagcacctattcaat
gagttgggtccgccaggctccaggggagggcctggaatggatcgccaccattaatactggtggtagcgcatactac
gcgaactgggcgaaaggccgattcaccatctccaaaacctcgaccacggtgactctgaaaatgaccagtctgacaa
ccgaggacacggccacctatttctgtgccagaacgattagtgctgataatttgtggggccacggcaccctggtcacc
gtctcctcagggcaacctaaggctccatcagtcttcccactggccccctgctgcggggacacacccagctccacggt
caccctgggttgtcttgtgaagggatacctcccggaacccgtgaccgtgacctggaactccggcaccctgaccaatg
gagtgcggaccttcccgagcgtcaggcagtcctccgggttgtacagcttgtctagcgtggtgtccgtgacgtcgtcaa
gccagcctgtgacttgcaatgtggcccatccggccaccaacaccaaggtcgacaagaccgtggcgccttccacctg
ttccaagcccacttgcccgccgcctgagctcctgggaggaccgtccgtgttcatcttccctccaaaacccaaggatac
cctgatgattagccgcactcccgaagtcacttgcgtggtcgtggacgtgtcgcag
Heavy chain (Clone #30)-amino acid (CDR1: bold; CDR 2: bold/italic; CDR3:
bold/italic/underlined)
METGLRWLLLVAVLKGVQCRSLEESGGRLVTPGTPLTLTCTVSGIDLSTY
SMSWVRQAPGEGLEWIAT [symbol] YYANWAKGRFTISKTSTTVTLKMT
SLTTEDTATYFC [symbol] WGHGTLVTVSSGQPKAPSVFPLAPCCGDTP
SSTVTLGCLVKGYLPEPVTVTWNSGTLTNGVRTFPSVRQSSGLYSLSSVV
SVTSSSQPVTCNVAHPATNTKVDKTVAPSTCSKPTCPPPELLGGPSVFIFPP
KPKDTLMISRTPEVTCVVVDVSQ
Light chain (Clone #30)-nucleic acid
atggacacgagggcccccactcagctgctggggctcctgctgctctggctcccaggtgccacatttgcccaagtgct
gacccagactccatcctccgtgtctgcagctgtgggaagcacagtcaccatcagttgccagtccagtcagagtgtttat
agtgactacttatcttggtatcagcagaaaccaggacagcctcccaagcgcctgatatatgctgcatttactctggcatc
tggtgtcccatcgcggttcaaaggcagtggatctgagaaacagttcactctcaccatcagcgacctggagtgtgacg
atgctgccacttactactgtgcagccatttatagtaatgatatttgggctttcggcggagggaccgaggtggtcgtcaaa
ggtgatccagttgcacctactgtcctcatcttcccaccatcagcggaccaagtggccactggcaccgtgaccattgtct
gcgtggctaacaagtacttcccggacgtgaccgtgacctgggaagtcgacggaaccactcagaccactggtatcga
gaacagcaagacgccccagaactccgccgattgtacttataacctgtcctccacactgaccctcacctcgacccagta
caattcccacaaggagtacacttgcaaagtcacccagggaaccacttcagtggtgcagagcttcaaccggggggatt
gctga
Light chain (Clone #30)-amino acid (CDR1: bold; CDR 2: bold/italic; CDR3:
bold/italic/underlined)
MDTRAPTQLLGLLLLWLPGATFAQVLTQTPSSVSAAVGSTVTISCQSSQS
VYSDYLSWYQQKPGQPPKRLIY [symbol] TLASGVPSRFKGSGSEKQFTLTISDL
ECDDAATYYC [symbol] FGGGTEVVVKGDPVAPTVLIFPPSADQVAT
GTVTIVCVANKYFPDVTVTWEVDGTTQTTGIENSKTPQNSADCTYNLSST
LTLTSTQYNSHKEYTCKVTQGTTSVVQSFNRGDC

The CDR structures of the heavy chain and light chain of Clone #30 provided above were predicted based on the alignment to the sequences of the germline clones using the IMGT® (the international ImMunoGeneTics information System®) software (http://www.imgt.org). The sequences between the CDRs are frame sequences. Thus, the above identified CDR sequences are the CDR residues and the frame residues may also associate with the immunogenic fragment or epitopes as antigen binding sites.

### Example 2: Identification of PIF Epitopes

To study PIF peptide and its derivatives of different length for binding to rabbit anti-PIF monoclonal antibody and rabbit anti-PIF polyclonal antibody, PIF peptides of various lengths as listed in Table 2 was synthesized, reconstituted in PBS and two different concentrations were tested by respective colorimetric ELISA following standard protocol. The standards were run in duplicate and the samples were run in triplicate and repeated three times.

TABLE 2
PIF peptides of various length for epitope mapping of anti-PIF antibody.

| AA | PIF-1 | N-terminal | | | | | | | PIF-1 |
|---|---|---|---|---|---|---|---|---|---|
| location | P1-15 | P5-10 | P8-13 | P10-15 | P8-15 | P1-7 | P1-13 | P1-14 | P1-15 |
| 1 | M | | | | | M | M | M | M |
| 2 | V | | | | | V | V | V | V |
| 3 | R | | | | | R | R | R | R |
| 4 | I | | | | | I | I | I | I |
| 5 | K | K | | | | K | K | K | K |
| 6 | P | P | | | | P | P | P | P |
| 7 | G | G | | | | G | G | G | G |
| 8 | S | S | S | | S | | S | S | S |
| 9 | A | A | A | | A | | A | A | A |
| 10 | N | N | N | N | N | | N | N | N |
| 11 | K | | K | K | K | | K | K | K |
| 12 | P | | P | P | P | | P | P | P |
| 13 | S | | S | S | S | | S | S | S |
| 14 | D | | | D | D | | | D | D |
| 15 | D | | | D | D | | | | D |

C-terminal

As shown in Table 3 below, the PIF colorimetric ELISA using rabbit polyclonal antibody showed 100% recovery to the full length PIF peptide (PIF-1 or P1-15), about 40% recovery to PIF peptide P1-13 and P1-14, and no peptide recovered for the rest of the shorter PIF peptides.

Similarly, the PIF colorimetric ELISA using rabbit anti-PIF monoclonal antibody showed 100% recovery to the full length PIF peptide (PIF-1 or P1-15), about 3.8% recovery to PIF peptide P1-14, and showed no reactivity to the rest of the peptides studied (Table 3).

TABLE 3
sPIF antigenic sequence determination by colorimetric
ELISA. The experiment was repeated three times
and a representative value shown here.

| | Spike | % recovery by | % Recovery by |
|---|---|---|---|
| Peptide | Concentration | Rabbit Polyclonal | Rabbit Monoclonal |
| (N-C) | (ng/ml) | Antibody | Antibody |
| P5-10 | 200 | 0 | 0 |
| | 100 | 0 | 0 |
| P8-13 | 200 | 0 | 0 |
| | 100 | 0 | 0 |
| P10-15 | 200 | 0 | 0 |
| | 100 | 0 | 0 |
| P8-15 | 200 | 0 | 0 |
| | 100 | 0 | 0 |
| P1-7 | 200 | 0 | 0 |
| | 100 | 0 | 0 |
| P1-13 | 200 | 31.1 | 0 |
| | 100 | 49.8 | 0 |
| P1-14 | 200 | 30.0 | 3.8 |
| | 100 | 44.6 | 3.8 |
| P1-15 | 1000 | 101.4 | 100.0 |
| | 500 | 115.0 | 100.0 |
| Scrambled PIF | 1000 | 0 | 0 |
| | 500 | 0 | 0 |

These results indicate that the rabbit anti-PIF monoclonal antibody is very specific to the full-length of PIF (PIF-1) and detects almost no other derivatives of PIF. By contrast, the rabbit anti-PIF polyclonal antibody binds to, in addition to the full-length PIF-1 (P1-15), P1-13 and P1-14, though at higher percentage to PIF-1 and only about 40% to P1-13 and P1-14.

### Example 3: Characterization of Binding-Specificity of Rabbit Anti-PIF Monoclonal Antibody Clone #30 to PIF

The PIF-binding specificity of the rabbit monoclonal antibody Clone #30 described in Example 1 was quantitatively characterized by surface plasmon resonance (SPR). SPR is an optical technique for detecting the interaction of two different molecules in which one is mobile and one is fixed on a thin gold film. In SPR analysis, affinity-purified ligand polypeptides are immobilized on a sensor chip inserted into the flow chamber of an instrument, such as Biacore 3000 instrument (Biacore, Uppsala, Sweden). Addition of a second polypeptide, the flow-through analyte, to the chamber, results in binding to the immobilized polypeptide ligand, producing a small change in refractive index at the gold surface, which can be quantified with precision. Binding affinities can be obtained from the ratio of rate constants, yielding a straightforward characterization of protein-protein interaction.

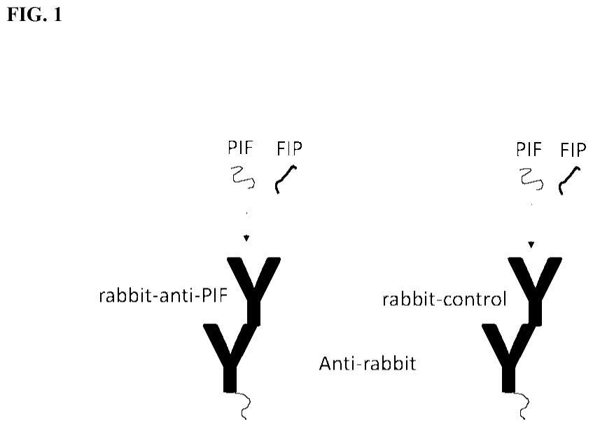

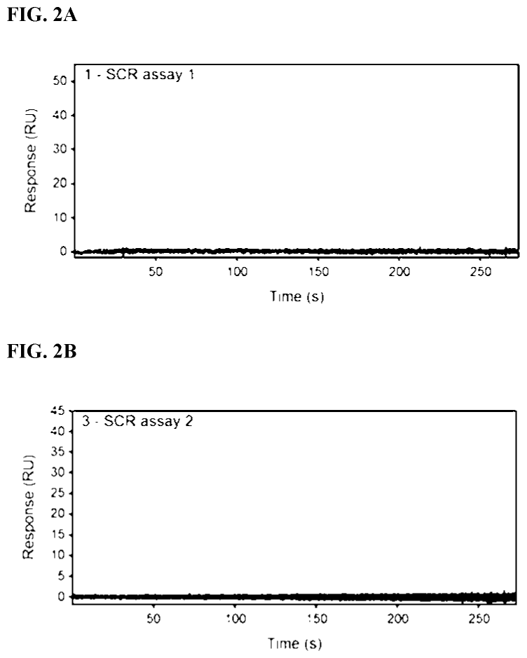

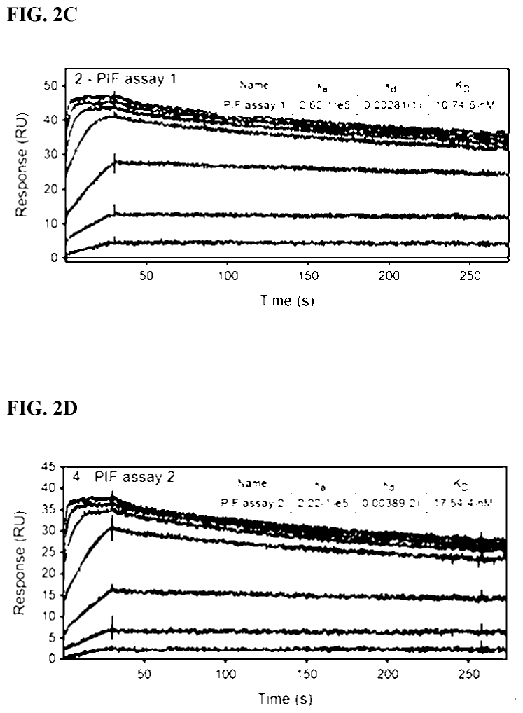

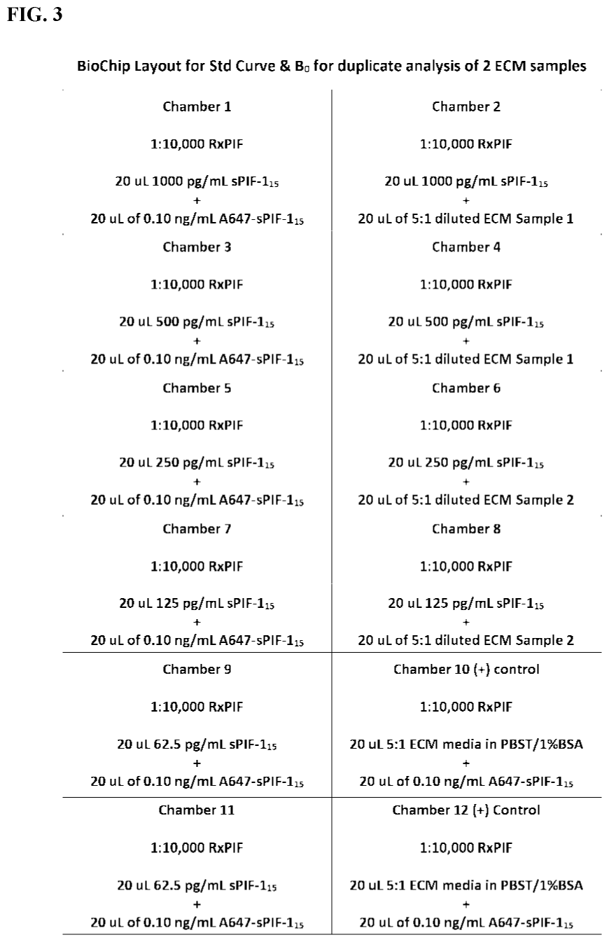

In the work described here, purified rabbit monoclonal antibody Clone #30 was used as the ligand polypeptide and PIF in solution was used as the analyte to evaluate their binding affinity. A schematic of this assay is presented in FIG. 1. To immobilize rabbit monoclonal antibody Clone #30 onto a chip, a sheep polyclonal anti-rabbit antibody was used. The sheep anti-rabbit antibody was amine coupled to a CM5 sensor chip using standard NHS/EDC activation. The sample Clone #30 was diluted 1/10 and captured onto one of the sheep anti-rabbit antibody coated flow cells up to about 2000 RU. PIF and scrambled PIF (SCR; containing scrambled amino acid sequence of PIF) peptides were tested in a 2-fold concentration series up to 10 μM. Each peptide was tested in duplicate. As shown in FIGS. 2A and 2B, no binding was observed for SCR. On the other hand, PIF showed a significant and concentration dependent binding response (FIGS. 2C and 2D). The PIF data globally fit to a 1:1 interaction model as shown by the gray lines in the figures. Under these conditions, the K_{D} for the PIF/Clone #30 interaction was about 15 nM.

### Example 4: Binding Specificity Analysis of Rabbit Anti-PIF Monoclonal Antibodies

The SPR technique described in Example 3 will be used to study the binding specificity of other rabbit anti-PIF monoclonal antibody candidates. A sheep polyclonal anti-rabbit antibody will be amine coupled to a chip such as CM5 sensor chip using standard NHS/EDC activation to capture a 1/10-diluted candidate rabbit anti-PIF monoclonal antibody up to about 2000 RU. PIF and scrambled PIF (SCR) peptides will be tested in a 2-fold concentration series up to about 10 μM. Each peptide will be tested at least duplicate. If needed, Clone #30 will be used to run a comparative analysis.

### Example 5: Evaluation of Competitive PIF Chemiluminescent ELISA on Embryo Culture Media Samples

The PIF chemiluminescent ELISA is based on a competitive binding principle. A labeled synthetic PIF (sPIF) is used as a tracer to compete with a non-labeled PIF present in a sample or standard using a constant and limited amount of anti-PIF antibody. A decreasing amount of tracer binds to the antibody as the amount of PIF in the ECM sample or standard is increased. Separation of the antibody-PIF complexes from free PIF is achieved by capture of the anti-PIF-tracer complex using a goat anti-rabbit capture antibody coated to the microplate.

A PIF chemiluminescent ELISA was performed on randomly selected 60 single embryo culture media samples from 26 patients. Fifty-nine out of 60 samples were in G-1 ™ Plus medium and one was in G-2 ™ Plus medium of the same patient. G-1 ™ Plus medium is to culture embryo from pronuclei to day 2 or 3, while G-2 ™ Plus medium is to culture embryo from day 3 to blastocyst stage. The negative control used in this evaluation was the blank G-1 ™ Plus medium. The detected PIF values of these samples are summarized in Table 4 below.

TABLE 4
Detected PIF values from 60 single embryo culture media
samples using competitive PIF chemiluminescent ELISA.

| | | | PIF Con- |
|---|---|---|---|
| Pa- | Sample | | centration |
| tient | No. | Sample ID | (ng/ml) |
| 1 | 1 | PT NR 102 PIF EMB 32218 G1- EMB 5 | 5.4 |
| | 2 | PT NR 102 PIF EMB 32218 G1 -EMB 21 | Not Detected |
| | 4 | PT 102 PIF EMB 32218 G1- EMB 11 | Not Detected |
| | 6 | PT NR 102 PIF EMB 32218 G1-EMB 13 | 6.4 |
| | 7 | PTNR 102 PIF EMB 32218 G1-EMB 16 | Not Detected |
| | 8 | PT NR 102 PIF EMB 32218 G1-EMB 18 | 4.6 |
| | 25 | PTNR-102-PIF EMB-32218-G1-EMB9 | 0.1 |
| | 28 | PTNR-102-PIF-EMB-32218-G1-EMB17 | 4.5 |
| 2 | 3 | PT NR 100 PIF EMB 32530 G1 -EMB2 | 1.9 |
| | 5 | PT NR 100 PIF EMB 32530G1- EMB-4 | 10.4 |
| | 27 | PTNR-100-PIF EMB-32530-G1-EMB5 | 5.4 |
| 3 | 9 | PTNR 89 PIF EMB 32152 G-EMB7 | 9.7 |
| | 29 | PT-NR-89-PIF-EMB-32152-G1-EMB3 | 15.4 |
| | 32 | PTNR-89-PIF-EMB-32152-G1-EMB8 | 15.2 |
| | 33 | PTNR-89-PIF-EMB-32152-G1-EMB9 | 6.5 |
| | 34 | PTNR-89-PIF-EMB-32152-G1-EMB11 | 2.1 |
| | 35 | PT-NR-89-PIF-EMB-32152-G1-EMB1 | 7.9 |
| 4 | 10 | 164 PIF EMB 31057-G1-7 | 5.2 |
| | 11 | 164 PIF EMB 31057-G1-9 | 8.3 |
| 5 | 12 | PTNR 93 PIF-EMB-32335-G1-EMB2 | 5.6 |
| 6 | 13 | PTNR-56-PIF EMB 30033-G1-EMB4 | 7.4 |
| 7 | 14 | 02078-2528-EMB-PIF-28586-G1-EMB2 | 9.3 |
| | 15 | 020781-2528-EMBPIF-28586-G1-EMB4 | 7.4 |
| 8 | 16 | PTNR61-PIFEMB-30129-G1-EMB1 | 3.2 |
| 9 | 19 | 280584-1624-EMB-PIF-28519G1-EMB8 | 7.0 |
| | 47 | 280584-1624-EMB-PIF-28519-G1-EMB3 | 17.7 |
| | 61 | 280584-1624-EMB-PIF-28519-G1-EMB4 | Not Detected |
| | 17 | 280584-1624 EMB PIF-28519G1-EMB6 | 1.4 |
| 10 | 18 | PTNR-56PIF-EMB-30033-G1-EMB1 | Not Detected |
| 11 | 22 | PTNR-91PIF-EMB-32287 G1-EMB1 | 19.9 |
| | 23 | PT NR91PIF-EMB-32287-G1-EMB2 | 14.6 |
| | 26 | PTNR-91-PIFEMB-32287-G1-EMB4 | 1.1 |
| 12 | 24 | PTNR-109-PIF-EMB-32756-G1-EMB3 | 9.2 |
| 13 | 30 | PTNR-86-PIF-EMB-31650-G1-EMB11 | 10.9 |
| 14 | 31 | PTNR-101-PIF-EMB-31932-G1-EMB1 | 7.1 |
| 15 | 36 | PTNR-98-PIF-EMB-31941-G1-EMB1 | 5.9 |
| 16 | 37 | PTNR-106-PIF-EMB-23727-G1-EMB1 | 10.2 |
| | 40 | PTNR-106-PIF-EMB-23727-G1-EMB15 | 9.2 |
| 17 | 38 | PTNR-92-PIF-EMB-32248-G1-EMB8 | 17.9 |
| | 49 | PTNR-92-PIF-EMB-32248-G1-EMB4 | 6.6 |
| | 50 | PTNR-92-PIF-EMB-32248-G1-EMB3 | 7.0 |
| | 42 | PTNR-92-PIF-EMB-32248-G1-EMB6 | 7.6 |
| | 43 | PTNR-92-PIF-EMB-32248-G1-EMB5 | Not Detected |
| | 51 | PTNR-92-PIF-EMB-32248-G1-EMB2 | Not Detected |
| 18 | 39 | PTNR-120-PIF-EMB-33109-G1-EMB2 | 16.6 |
| 19 | 41 | PTNR-111-PIF-EMB-32659-G1-EMB15 | 12.1 |
| | 44 | PTNR-111-PIFEMB-32659-G1-EMB14 | 12.6 |
| | 52 | PTNR-111-PIF-EMB-32659-G1-EMB8 | 8.6 |
| 20 | 45 | 020781-2528EMB-PIF-285586-G1-EMB3 | 15.4 |
| 21 | 46 | 250786-1582-PIF-EMB-28504-G1-EMB5 | 12.4 |
| 22 | 48 | PTNR-87-PIF-EMB-31582-G2-EMB1 | 12.0 |
| | 58 | PTNR-87-PIF-EMB-31582-G1-EMB3 | 7.8 |
| 23 | 53 | PTNR-82-PIF-EMB-31118-G1-EMB2 | 5.7 |
| 24 | 54 | PTNR-X-EMBPIF-30677-G1-EMB8 | 8.5 |
| | 56 | PTNR-X-EMB-PIF-30677-G1-EMB10 | 4.4 |
| | 57 | PTNR-X-EMB-PIF-30677-G1-EMB11 | 3.8 |
| | 59 | PTNR-X-EMB-PIF-30677-G1-EMB3 | 0.7 |
| | 60 | PTNR-X-EMB-PIF-30677-G1-EMB4 | 3.5 |
| 25 | 55 | PTNR-PIF-EMB-30426-G1-EMB9 | 11.0 |
| 26 | 62 | 230578-2656-EMB-PIF-28581-G1-EMB6 | 5.3 |

Among all the samples tested, PIF was detected in 53 out of 60 samples. The highest and lowest amounts of PIF were 19.9 and 0.1 ng/ml, respectively, with a mean amount of 8.2 ng/ml. A breakdown of this evaluation is as follows:

| | |
|---|---|
| Total Patients (n) | 26 |
| Total Samples (n) | 60 |
| PIF detected (n) | 53 |
| Not detected (n) | 7 |
| Median PIF (ng/ml) | 7.4 |
| Mean PIF (ng/ml) | 8.2 |
| High PIF (ng/ml) | 19.9 |
| Low PIF (ng/ml) | 0.1 |
| % Detected | 88.3 |
| % Not Detcetd | 11.7 |

### Example 6: Microarray-Based Immunoassay for PIF Detection in Day 5 and Day 6 Spent Embryo Culture Medium (ECM) Samples

The microarray-based immunoassay for PIF detection is essentially a micro-ELISA which is based on a similar competitive ELISA principle described in Example 5. Here, a fluorescently labeled PIF is used as a tracer to compete with a non-labeled PIF present in a sample or standard using a constant and limited amount of anti-PIF antibody, such as rabbit anti-PIF monoclonal antibody. A decreasing amount of tracer binds to the antibody as the amount of PIF in the sample or standard is increased. Separation of the antibody-PIF complexes from free PIF is achieved by capture of the anti-PIF-tracer complex using a species-specific capture antibody bound to the microarray surface.

Materials and Methods

A Barnstead/Thermoline Waxer heater was fitted with a 12-chamber mold and heated to 130° C., and high purity paraffin wax was added until the reservoir was filled with the melted wax. The biochips were then waxed and stored in a dessicator at <20% RH (Relative Humidity) until ready for use. Each chamber in this 12-chamber format of biochips has a capacity of 40 μL.

A 1 mg/mL substrate solution of a goat anti-Rabbit (GxR) polyclonal antibody (Thermo-Fisher Scientific) was prepared by dissolving the substrate in a solution of 40% glycerol/PBS (Phosphate-Buffered Saline) for spotting. A block of 20 spots of the GxR substrate solution was printed into each chamber using a Genetix QArray-2 microarray printer (Molecular Devices) using 150 μm capillary pins and the printed array stored in <20% RH for 4-7 days prior to use.

The GxR spotted biochip was carefully dipped horizontally face up in PBS+0.5% Tween (PBST) and then blocked in a freshly prepared 1% BSA in PBST (B-PBST) for 1-2 hours at room temperature using a Coplan jar and then rinsed with PBST and air dried. Using a B-PBST solution, a 1:10,000 dilution of a 0.32 mg/mL solution of Rabbit anti-PIF-1₁₅ (RxPIF-1₁₅) monoclonal antibody (Clone #30 described in Example 1; ExonBio/BioIncept) was prepared and μL was pipetted into each chamber and the biochip was incubated for 1-2 hours at room temperature. The PIF-1₁₅ peptide is the synthetic, full-length PIF peptide (sPIF-1₁₅) described previously. Using a vacuum aspirator, the RxPIF-1₁₅ solution was carefully removed from the chambers and the biochip was washed with PBST and air dried.

A 1000 μg/mL solution of the sPIF-1₁₅ peptide (BioSynthesis) was prepared in B-PBST and 5 serial dilutions of a factor of 2 were generated from this solution for a total of 6 peptide standards: 1000 pg/mL, 500 pg/mL, 250 pg/mL, 125 pg/mL, 62.5 pg/mL, 31.25 pg/mL. A 100 pg/mL solution of N-terminus labeled Alexafluor 647-sPIF-1₁₅ peptide (A647-sPIF) (BioSynthesis) was prepared and an equal volume of this solution was added to each of the standards prepared and thoroughly mixed. 40 μL of each mixture prepared was carefully pipetted into 6 separate chambers (one standard per chamber), leaving 6 chambers for the analysis of samples and controls. 10:1 diluted ECM samples for duplicate analysis were prepared by dissolving 8 μL of sample in 32 μL of B-PBST and then adding 40 μL of the 100 pg/mL A647-sPIF solution prepared and thoroughly mixed. 40 μL of each ECM sample containing the A647-sPIF solution was added to a separate chamber. Negative control (B₀) was prepared by dissolving 8 μL of the ECM media used to culture the embryos in 32 μL of B-PBST and then adding 40 of the 100 pg/mL A647-sPIF solution. The biochip was then incubated for 1-2 hours in the dark. Using a vacuum aspirator, the solution in the chambers was carefully removed from the chambers and the biochip was washed with PBST and briefly rinsed with distilled water and air dried.

A representative biochip layout for standard curve and B₀ value for duplicate analysis of 2 ECM samples is provided in FIG. 3. Because this is a competitive assay, it is important to note that the concentrations and ratios of the different reagents used in this assay reflect the expected amount of the peptide in the samples being tested. If the samples have higher levels of the peptide antigen, then higher levels of the labeled and unlabeled peptide used in the assay will need to be adjusted accordingly. The example provided herein represents the levels of labeled and unlabeled peptide required to measure the peptide in ECM samples. It is also not important which chamber contain which solution used in the analysis.

The assayed biochips were analyzed with a fluorescence microarray reader and analysis software (Innopsys SA) to generate the data files containing the signal intensities for each spot in each chamber. The 20 spots were then averaged and tabulated for standard curve analysis and generation of the curve fit equation to calculate of the peptide levels in the ECM sample. All Data were normalized to the B₀ RFU (Relative Fluorescence Unit) values obtained. 4PL and log curve fit equations were generated and the results for the ECM samples were tabulated.

Other fluorescence readers and analysis software known by one skilled in the art can be used to generate the signal intensities for spot analysis.

Results

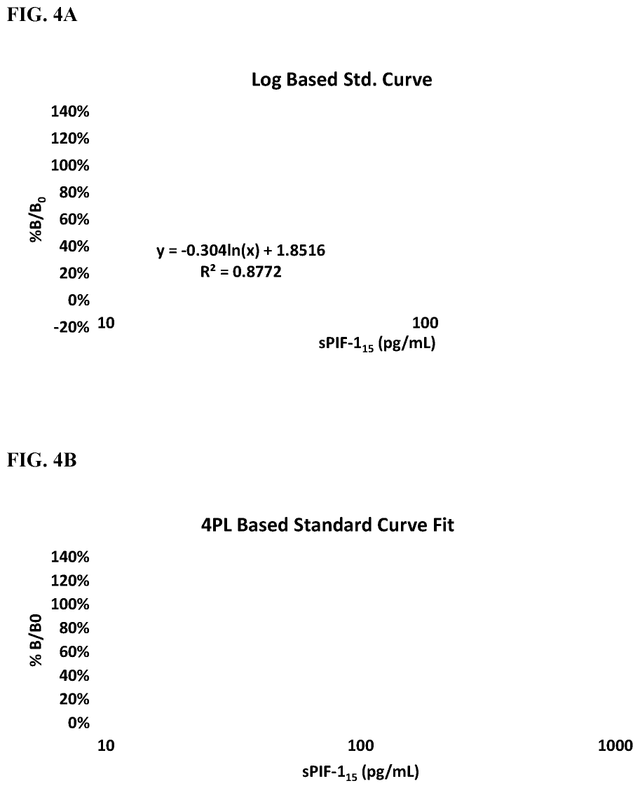

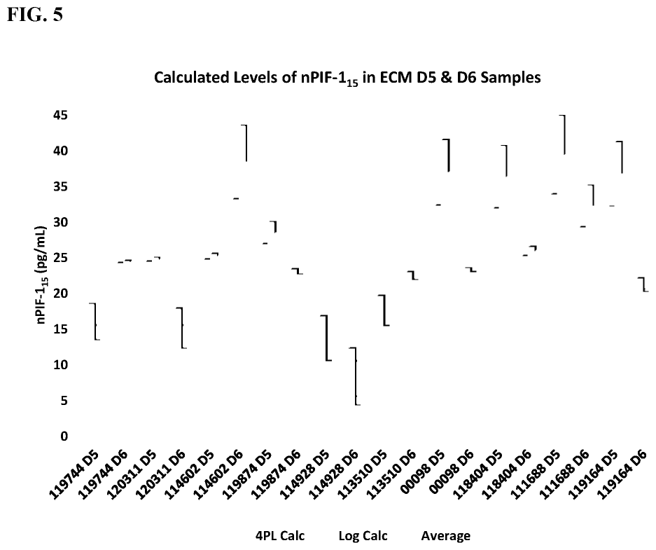

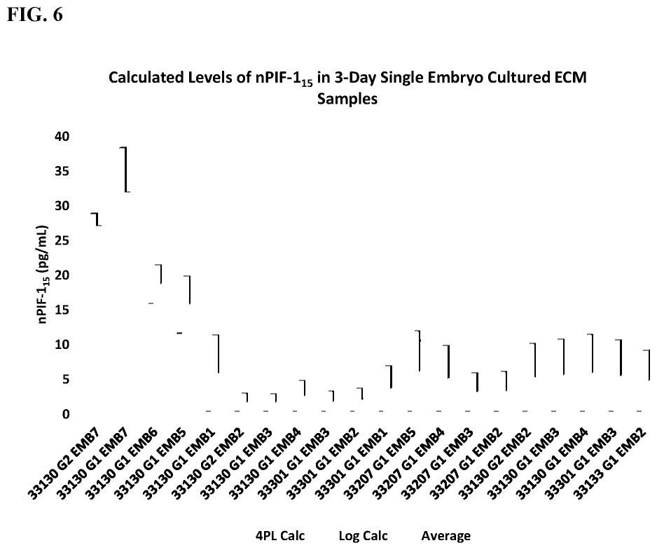

Using the materials and methods described above, 20 ECM samples obtained from spent embryo culture medium containing multiple embryos at 5 and 6 days post culturing (D5 samples and D6 samples) as well as controls at 5 and 6 days (no embryo present) were analyzed for nPIF-1₁₅ levels. Several 12-chamber biochips were used for this analysis with a standard curve and Absolute controls run on each biochip. The Log based curve fit and the 4PL curve fit were generated. The Log based curve fit calculates a best fit to indicate the linearity of the data, while the 4PL curve fit is calculated (MyCurveFit.com) with best fit linearity trendline identical to the Log curve fit. The average mean RFU value for the Absolute control was used for B₀ to calculate % B/B₀ for the data set. All data were then normalized to the Absolute Control samples. The standard curves obtained from the Log and 4PL based curve fit calculations are provided in FIG. 4A and FIG. 4B, respectively. Samples were run in duplicate on separate biochips and the results shown are inter-slide averages. The data obtained from the controls and the ECM samples are provided in Tables 5, 6 and 7.

TABLE 5
Calculated data of the positive controls (ABS: Absolute Control).

| Standard | nPIF-1₁₅ | Average | | Standard | |
|---|---|---|---|---|---|
| Curve | (pg/mL) | Mean | % B/B₀ | Deviation | CV's |
| Chamber 1 | 500 | 285 | 3% | 38 | 13% |
| Chamber 3 | 250 | 1694 | 19% | 335 | 20% |
| Chamber 5 | 125 | 3849 | 43% | 804 | 21% |
| Chamber 7 | 62.5 | 3353 | 38% | 683 | 20% |
| Chamber 9 | 31.25 | 6170 | 69% | 991 | 16% |
| Chamber 11 | 15.625 | 10866 | 122% | 2011 | 19% |
| Chamber 12 | ABS | 8929 | | | |

TABLE 6
Calculated data for the negative controls.

nPIF-1₁₅ (pg/mL)

| Sample ID | D5 Controls | D6 controls |
|---|---|---|
| 121515 | 5.4 | 22.9 |
| 120559 | 2.1 | 2.8 |
| 116050 | 1.0 | 2.8 |
| 121990 | 0.9 | 12.1 |
| 111374 | 3.3 | 7.3 |

TABLE 7
Calculated data of the ECM samples.

| % CV | Standard Deviation | Sample I.D. | Average RFU's | % B/B₀ |
|---|---|---|---|---|
| 13% | 1233 | 119744 D 6 | 9530 | 107% |
| 1% | 78 | 119744 D 5 | 7877 | 88% |
| 2% | 172 | 120311 D 6 | 7829 | 88% |
| 14% | 1327 | 120311 D 5 | 9782 | 110% |
| 7% | 532 | 114602 D 6 | 7769 | 87% |
| 52% | 3293 | 114602 D 5 | 6310 | 71% |
| 6% | 419 | 119874 D 6 | 7329 | 82% |
| 12% | 942 | 119874 D 5 | 8091 | 91% |
| 16% | 1625 | 114928 D 6 | 10200 | 114% |
| 9% | 1210 | 114928 D 5 | 12736 | 143% |
| 1% | 83 | 113510 D 6 | 9138 | 102% |
| 8% | 694 | 113510 D 5 | 8187 | 92% |
| 1% | 49 | 00098 D 6 | 6438 | 72% |
| 18% | 1465 | 00098 D 5 | 8057 | 90% |
| 7% | 481 | 118404 D 6 | 6496 | 73% |
| 12% | 916 | 118404 D 5 | 7666 | 86% |
| 34% | 2111 | 111688 D 6 | 6227 | 70% |
| 26% | 1771 | 111688 D 5 | 6899 | 77% |
| 20% | 1314 | 119164 D 6 | 6459 | 72% |
| 18% | 1475 | 119164 D 5 | 8408 | 94% |

After normalization, the calculated levels of nPIF-1₁₅ in each ECM sample analyzed are summarized in Table 8 below with a bar chart presented in FIG. 5. The agreement of the Log fit of the raw RFU data and the 4PL curve fitted data indicates that the data set is internally consistent, and the results are indicative of the basic reliability of the data and assay performance. The bar chart presented in FIG. 5 shows the comparison data for the Log based and 4PL based calculations of the mean RFU values for the 20 samples analyzed. The light-gray bars are the average of the 2 calculation models. The LOD for the measured values of the peptide in the assay is 10-20 pg/mL highlighting that the embryo containing samples are clearly showing elevated levels of the peptide compared to the controls.

TABLE 8
The calculated levels of nPIF-1₁₅ in each ECM sample analyzed.

| | 4PL | | Log | |
|---|---|---|---|---|
| | Calculation | | Calculation | |
| | nPIF-1₁₅ | | nPIF-1₁₅ | |
| Sample ID | (pg/mL) | Average | (pg/mL) | Sample ID |
| 119744 D 5 | 18 | 16 | 13 | 119744 D 5 |
| 119744 D 6 | 24 | 24 | 24 | 119744 D 6 |
| 120311 D 5 | 24 | 24 | 25 | 120311 D 5 |
| 120311 D 6 | 18 | 15 | 12 | 120311 D 6 |
| 114602 D 5 | 24 | 25 | 25 | 114602 D 5 |
| 114602 D 6 | 33 | 38 | 43 | 114602 D 6 |
| 119874 D 5 | 27 | 28 | 30 | 119874 D 5 |
| 119874 D 6 | 23 | 23 | 22 | 119874 D 6 |
| 114928 D 5 | 17 | 13 | 10 | 114928 D 5 |
| 114928 D 6 | 12 | 8 | 4 | 114928 D 6 |
| 113510 D 5 | 19 | 17 | 15 | 113510 D 5 |
| 113510 D 6 | 23 | 22 | 22 | 113510 D 6 |
| 00098 D 5 | 32 | 37 | 41 | 00098 D 5 |
| 00098 D 6 | 23 | 23 | 23 | 00098 D 6 |
| 118404 D 5 | 32 | 36 | 40 | 118404 D 5 |
| 118404 D 6 | 25 | 26 | 26 | 118404 D 6 |
| 111688 D 5 | 34 | 39 | 45 | 111688 D 5 |
| 111688 D 6 | 29 | 32 | 35 | 111688 D 6 |
| 119164 D 5 | 32 | 36 | 41 | 119164 D 5 |
| 119164 D 6 | 22 | 21 | 20 | 119164 D 6 |

### Example 7: Microarray-Based Immunoassay for PIF Detection in Day 3 Spent Single Embryo Culture Medium (ECM) Samples

To test whether nPIF-1₁₅ peptide can be detected in single embryo culture media, a series of ECM samples prepared by culturing single embryos and harvested after 3 days were obtained and analyzed using the method described in Example 6. Using the 20 pg/mL cut-off value, it is apparent that the peptide can be detected as early as 3 days in a single embryo culture, although the sensitivity is not as high. The calculated levels of nPIF-1₁₅ in each ECM sample analyzed are summarized in Table 9 below with a bar chart presented in FIG. 6.

TABLE 9
The calculated levels of nPIF-1₁₅ in each
single-embryo ECM sample analyzed.

| | 4PL Calc | | Log Calc |
|---|---|---|---|
| Sample I.D. | nPIF-1₁₅ (pg/mL) | Average | nPIF-1₁₅ (pg/mL) |
| 33130 G2 EMB7 | 29 | 28 | 27 |
| 33130 G1 EMB7 | 38 | 35 | 32 |
| 33130 G1 EMB6 | 16 | 18 | 21 |
| 33130 G1 EMB5 | 11 | 15 | 20 |
| 33130 G1 EMB1 | 0 | 6 | 11 |
| 33130 G2 EMB2 | 0 | 1 | 3 |
| 33130 G1 EMB3 | 0 | 1 | 3 |
| 33130 G1 EMB4 | 0 | 2 | 4 |
| 33301 G1 EMB3 | 0 | 1 | 3 |
| 33301 G1 EMB2 | 0 | 2 | 3 |
| 33301 G1 EMB1 | 0 | 3 | 7 |
| 33207 G1 EMB5 | 0 | 6 | 12 |
| 33207 G1 EMB4 | 0 | 5 | 10 |
| 33207 G1 EMB3 | 0 | 3 | 6 |
| 33207 G1 EMB2 | 0 | 3 | 6 |
| 33130 G2 EMB2 | 0 | 5 | 10 |
| 33130 G1 EMB3 | 0 | 5 | 11 |
| 33130 G1 EMB4 | 0 | 6 | 11 |
| 33301 G1 EMB3 | 0 | 5 | 10 |
| 33133 G1 EMB2 | 0 | 4 | 9 |

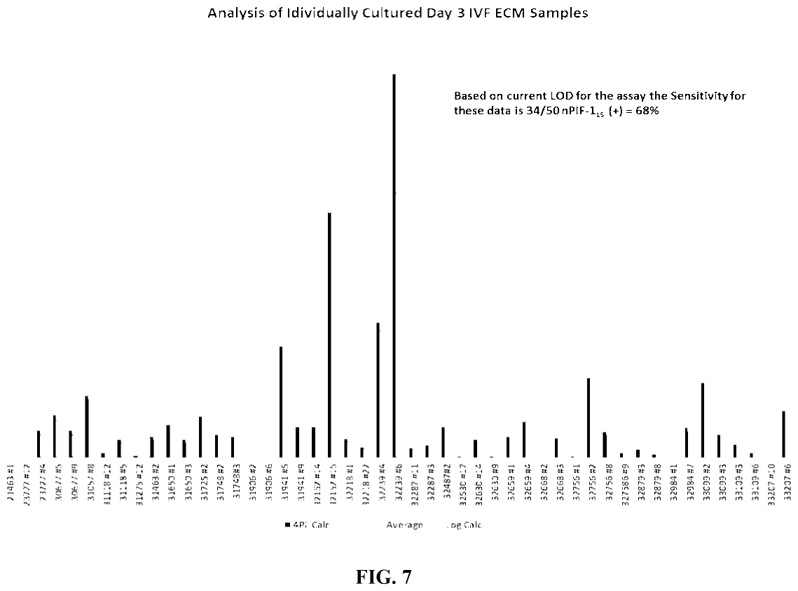

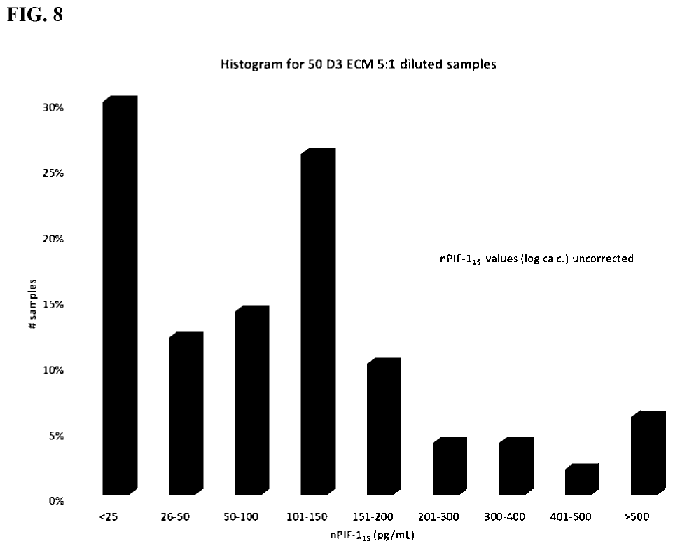

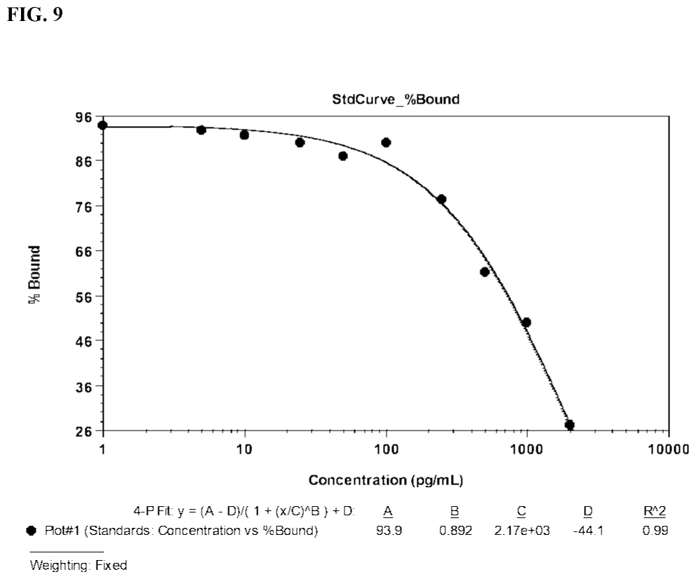

Another 50 spent ECM samples harvested at Day 3 were obtained and analyzed using the method described in Example 6. The calculated levels of nPIF-1₁₅ in each ECM sample analyzed are summarized in FIG. 7. Thirty-four out of the 50 samples (about 65%) tested showed measurable PIF at Day 3 with a limit of detection of 20 pg/mL. The histogram data for these 50 samples indicating the number of samples in each measured level of nPIF-1₁₅ is provided in FIG. 8. These data indicate that the majority of Day 3 ECM samples will be below 500 pg/mL which is where the assay range was optimizing to.

Based on these data, it appears that 5:1 dilution should be the maximum recommended dilution for the microarray assay because higher dilutions will likely cause a significant drop in the number of samples with measurable levels of nPIF-15. About 85% of the samples tested at the dilution had nPIF-1₁₅ measured values of <200 pg/mL (not dilution corrected). This means that the dilution corrected values for nPIF-1₁₅ concentrations in the ECM is <1000 pg/mL (e.g., <1 ng/mL) for the vast majority of the samples in the microarray assay. This is far lower than what has been reported in the past for Day 3 single embryo cultures (dilution corrected), such as the data reported in Example 5 using the competitive PIF chemiluminescent ELISA. assay.

To calculate the concentration of nPIF-1₁₅ in the ECM samples for the competitive microarray assay, the Log based linear curve fit is considered to be more accurate formula than the 4PL curve fit. It is a linear curve fit equation which does not skew the high or low end of the working range of the assay defined by the Standard Curve. The 4PL curve fit, on the other hand, can skew the high and low range in order to provide a best curve fit, but values close to these high and low limits can have higher errors associated with them. Both curve fit functions can be used to check the quality of the data when reporting data, but the Log based curve fit results should be used when reporting actual numbers.

Analysis of the Standard Curves showed CV's<20% for the standards, and for Blank (B₀) values for the G1+ Medium, a CV of <10% indicates good assay performance and inter-slide variability. The working range of 500 pg/mL to 10 pg/mL is the linear range for the microarray assay standard of operation for ECM analysis. No special “reagents” are needed to provide CV's<20%. Pipetting accuracy is the critical component to getting reproducible results because of the low concentrations required for the linear standard curve range to measure nPIF-1₁₅ in ECM.

Going forward, the “error bars” for the reported value of nPIF-1₁₅ in the ECM sample with the assay will be determined. Whether the spot density (Print spacing) on the microarray has an impact on improving the assay robustness will also be tested. The stability of the solutions used for generating standard curve as well as diluted, labeled A647-sPIF-1₁₅ solutions for routine assay operation will need to be established, which includes determination of whether the solutions can be prepared and used multiple times, what is the best storage conditions, and how long do the solutions last. Other format of the assay and the ability to automate the assay will also be investigated in order to handle much higher sample throughput.

### Example 8: Competitive ELISA Assay for PIF Detection in Day 3 Spent Single Embryo Culture Medium (ECM) Samples

The ability of the rabbit monoclonal antibody Clone #30 described in Example 1 to detect nPIF-1₁₅ peptide in spent embryo culture media using a competitive ELISA assay was tested. A series of ECM samples prepared by culturing single embryos and harvested after 3 days were obtained and analyzed. The rabbit monoclonal antibody Clone #30 was immobilized onto a BIC coated plate via a goat anti-rabbit polyclonal antibody at a concentration of 2 μg/mL. A biotin labeled PIF is used as a tracer to compete with a non-labeled PIF present in a sample or control.

Five sPIF-1₁₅ peptide solutions at different concentration were prepared as positive controls: 10 pg/mL, 1 pg/mL, 250 pg/mL, 25 pg/mL and 500 pg/mL. Ten standard samples with sPIF-1₁₅ peptide at different concentration were prepared to generate a standard curve: 1 pg/mL, 5 pg/mL, 10 pg/mL, 25 pg/mL, 50 pg/mL, 100 pg/mL, 250 pg/mL, 500 pg/mL, 1000 pg/mL, and 2000 pg/mL. The data obtained from the controls, standard samples, and the ECM samples are provided in Tables 10, 11 and 12 with the generated standard curve provided in FIG. 9.

TABLE 10
Calculated data of the positive controls.

| | | | | Result | | |
|---|---|---|---|---|---|---|
| Control | Values | MeanValues | AvgNetRLU | (pg/mL) | CV % | % Bound |
| 10 pg/mL | 27889503.97 | 27449578.22 | 22251687.58 | 2.386 | 2.3 | 93.611 |
| | 27009652.47 | | | | | |
| 1 pg/mL | 28624063.54 | 28153510.05 | 22955619.4 | Range? | 2.4 | 96.572 |
| | 27682956.55 | | | | | |
| 250 pg/mL | 24784711.52 | 24540448.95 | 19342558.3 | 164.229 | 1.4 | 81.372 |
| | 24296186.37 | | | | | |
| 25 pg/mL | 27173675.42 | 26841579.57 | 21643688.92 | 28.945 | 1.7 | 91.053 |
| | 26509483.72 | | | | | |
| 500 pg/mL | 22266655.48 | 21803442.52 | 16605551.88 | 379.412 | 3 | 69.858 |
| | 21340229.56 | | | | | |

TABLE 11
Calculated data of the standard samples.

| Sample | Conc. | Values | MeanValue | Std. Dev. | CV % | AvgNetRLU | % Bound |
|---|---|---|---|---|---|---|---|
| St01 | 2000 | 11871448 | 11612504 | 366202 | 3.2 | 6414614.00 | 27 |
| | | 11353561 | | | | | |
| St02 | 1000 | 17492271 | 17044499 | 633246 | 3.7 | 11846608.00 | 49.8 |
| | | 16596726 | | | | | |
| St03 | 500 | 20321161 | 19729615 | 836572 | 4.2 | 14531725.00 | 61.1 |
| | | 19138070 | | | | | |
| St04 | 250 | 23706412 | 23564158 | 201178 | 0.9 | 18366267.00 | 77.3 |
| | | 23421904 | | | | | |
| St05 | 100 | 26909922 | 26570739 | 479678 | 1.8 | 21372849.00 | 89.9 |
| | | 26231556 | | | | | |
| St06 | 50 | 26430510 | 25876932 | 782877 | 3 | 20679042.00 | 87 |
| | | 25323355 | | | | | |
| St07 | 25 | 26622376 | 26587445 | 49400 | 0.2 | 21389555.00 | 90 |
| | | 26552514 | | | | | |
| St08 | 10 | 27223287 | 26991934 | 327183 | 1.2 | 21794043.00 | 91.7 |
| | | 26760581 | | | | | |
| St09 | 5 | 27514884 | 27255434 | 366918 | 1.3 | 22057543.00 | 92.8 |
| | | 26995984 | | | | | |
| St10 | 1 | 27697131 | 27476409 | 312148 | 1.1 | 22278518.00 | 93.7 |
| | | 27255687 | | | | | |
| St11 | 0 | 28629632 | 28013787 | 870937 | 3.1 | 22815896.00 | 96 |
| | | 27397941 | | | | | |

TABLE 12
Calculated data of the ECM samples.

| | | | | Result | Dil. | Final Conc | | % |
|---|---|---|---|---|---|---|---|---|
| Sample | Values | MeanValues | AvgNetRLU | (pg/mL) | Factor | (pg/mL) | CV % | Bound |
| 28709 | 26807155 | 26807155 | 21609264.35 | 30.622 | 8.3 | 254.16 | 0 | 90.908 |
| EMB 10 | | | | | | | | |
| 28709 | 26725143.52 | 26725143.52 | 21527252.88 | 34.67 | 6.7 | 232.29 | 0 | 90.563 |
| EMB 18 | | | | | | | | |
| 28709 | 27621701.07 | 27621701.07 | 22423810.42 | Range? | 6.7 | #VALUE! | 0 | 94.335 |
| EMB 2 | | | | | | | | |
| 28709 | 26836010.89 | 26836010.89 | 21638120.24 | 29.215 | 6.7 | 195.74 | 0 | 91.03 |
| EMB 23 | | | | | | | | |
| 28709 | 27105838.76 | 27105838.76 | 21907948.12 | 16.576 | 6.7 | 111.06 | 0 | 92.165 |
| EMB 7 | | | | | | | | |
| 28741 11 | 27850016.97 | 27850016.97 | 22652126.32 | Range? | 6.7 | #VALUE! | 0 | 95.295 |
| 28741 | 27603476.3 | 27603476.3 | 22405585.65 | Range? | 6.7 | #VALUE! | 0 | 94.258 |
| EMB 2 | | | | | | | | |
| 28890 | 26224974.77 | 26224974.77 | 21027084.13 | 60.829 | 6.7 | 407.55 | 0 | 88.459 |
| EMB 2 | | | | | | | | |
| 30265 | 27542220.81 | 27542220.81 | 22344330.17 | Range? | 6.7 | #VALUE! | 0 | 94.001 |
| EMB 2 | | | | | | | | |
| 30282 | 26865879.26 | 26865879.26 | 21667988.62 | 27.77 | 6.7 | 186.06 | 0 | 91.155 |
| EMB 12 | | | | | | | | |
| 30282 | 26769186.72 | 26769186.72 | 21571296.08 | 32.487 | 6.7 | 217.66 | 0 | 90.748 |
| EMB 2 | | | | | | | | |
| 30282 | 27600945.08 | 27600945.08 | 22403054.43 | Range? | 6.7 | #VALUE! | 0 | 94.248 |
| EMB 6 | | | | | | | | |
| 30282 G1 | 27584239.04 | 27584239.04 | 22386348.39 | Range? | 6.7 | #VALUE! | 0 | 94.177 |
| EMB 5 | | | | | | | | |
| 30282 G2 | 26723118.55 | 26723118.55 | 21525227.9 | 34.771 | 6.7 | 232.97 | 0 | 90.555 |
| EMB 5 | | | | | | | | |
| 30298 | 26901316.32 | 26901316.32 | 21703425.68 | 26.069 | 6.7 | 174.66 | 0 | 91.304 |
| EMB 3 | | | | | | | | |
| 30298 | 26993452.67 | 26993452.67 | 21795562.02 | 21.721 | 8.3 | 180.28 | 0 | 91.692 |
| EMB 4 | | | | | | | | |
| 30298 | 27616638.63 | 27616638.63 | 22418747.99 | Range? | 6.7 | #VALUE! | 0 | 94.314 |
| EMB 8 | | | | | | | | |
| 30308 | 27382754.05 | 27382754.05 | 22184863.41 | 4.881 | 6.7 | 32.70 | 0 | 93.33 |
| EMB 13 | | | | | | | | |
| 30308 | 26800573.83 | 26800573.83 | 21602683.18 | 30.944 | 6.7 | 207.32 | 0 | 90.881 |
| EMB 14 | | | | | | | | |
| 30308 | 26701350.07 | 26701350.07 | 21503459.42 | 35.858 | 8.3 | 297.62 | 0 | 90.463 |
| EMB 8 | | | | | | | | |

### Example 9: Standard Operating Procedure for Detection of PIF in Embryo Culture

One of the most, if not the most, difficult clinical question facing the IVF medical community is which embryo to select for transfer. Evaluation of embryo morphology, rate of development and preimplantation genetic testing results all play important roles in selecting the most likely embryo to implant. Even with the use of these parameters, embryo selection is imprecise. Embryo implantation rates for clinics across the United States remain at less than 50% for young patients (<35 years old). For older patients (>40 years old), less than 20% of embryos transferred implant and the live birth rate per IVF cycle for this age group is less than 10%.

In an effort to better the select the embryo for transfer and improve implantation rates, scientists have been looking at the contents of spent culture media. The hypothesis has been that there is a peptide secreted by the developing embryo that can be used as a predictive marker of embryo viability to implant. We present the hypothesis that this peptide marker can be PIF. PIF is an early biomarker of pregnancy. Early data has shown that not all embryos in culture produce PIF and that embryos that do not produce PIF do not implant and do not produce Human Chorionic Gonadotropin (HCG) after transfer. It is our hypothesis that PIF may be a significant non-invasive peptide marker that will allow the IVF medical community to avoid choosing a non-viable embryo for transfer.

Hypothesis

Preliminary studies indicate that a proportion of in vitro derived human embryos may not produce PIF. It is hypothesized that embryos which do not produce PIF are unable to implant and establish a pregnancy.

Objective

The objective of this project is to collect spent culture media from single embryo culture to determine the relative PIF levels for developing individual embryos. The concentration or absence of PIF will then be evaluated with each aligned blastocyst embryo and the embryo's actual implantation or lack thereof. This could be the first non-invasive method to evaluate the ability of a blastocyst embryo to implant prior to transfer.

Inclusion Criteria
Patient age: all ages included (including egg donors)
PGT-A or untested embryos
Cycles with normally fertilized oocytes

Exclusion Criteria
PGT-M & PGT-SR cases
Patients with uterine factor infertility
Gestational carrier cycles
Endometrial lining at time of fresh or frozen embryo transfer<7.5 mm (data for endometrial lining of all embryo transfers will be collected, but those <7.5 mm to be excluded from analysis)

Materials Provided by IVF Laboratory:
Embryo culture dishes
Embryo culture media
Pipettors
Pipet tips

Embryo Culture
1. Oocyte retrieval, insemination (IVF or ICSI) and embryo culture are to be performed by the IVF Laboratory's clinical routine.
   a. For IVF laboratories culturing individual embryos in 25 μL drops and refreshing culture media on Day 3, spent culture media will be collected Day 3 & Day 5, 6 or 7:
      i. On Day 3, after the embryos have been moved to fresh culture drops, carefully withdraw 20 μL of the volume of the culture media drop per the sample collection instructions below.
      ii. When culture is completed on Day 5, 6 or 7, and all progressing embryos have been transferred or frozen, collect 20 μL of the volume of the culture media drop per the sample collection instructions below.
   b. For IVF laboratories which are single embryo culturing continuously in 25 only Day 5, 6 or 7 spent culture media will be collected:
      i. When culture is completed on Day 5, 6 or 7, and all progressing embryos have been transferred or frozen, collect 20 μL of the volume of the culture media drop per the sample collection instructions below.

Spent Media Collection

Provided Materials
0.5 ml micro packaging vials
Coded tube labels
5×5 cryovial racking box with lid
Biopsy worksheet
Styrofoam cooler and shipping box

Labeling Procedure
1. Ensure that materials and forms for only one patient are in the working space.
2. Place the provided bolded label on the biopsy worksheet and one on the cryovial box.
3. The appropriately numbered provided labels will be used for each sample and one negative control. The labels should be placed on the tubes before media drop collection.
   a. Place the label around the tube, as close to the top of the tube as possible.
   b. Check to ensure the labels on the tubes match the label placed on the media collection worksheet.
4. Label the top of each tube with the corresponding embryo number. 6 vs 9 should be clarified with an underline. Label a single negative control sample with the letters “NC.”

Sample Preparation and Collection
1. Wear powder free gloves for tube handling, labeling and media loading.
2. Sample preparation and collection should be performed in a working laminar flow hood.
3. Pipet tips should be changed between samples.
4. Using an appropriate pipettor (Gilson, Rainin, Eppendorf P20, P100 or P200), aspirate μL of spent culture media into the pre-labelled microcentrifuge tube. Note: In order to cleanly collect the 20 μL of spent culture media without contaminating with the oil overlay, hold the pipettor relatively vertically in the center of the drop when aspirating the media.
5. Complete the “Media Drop Collection Worksheet” including the embryo numbers, stage and morphology. Blastocyst morphology grading is to be recorded according to Gardner's criteria.

Sample Packaging & Transport
1. After all media samples are collected, close and securely tape the rack to ensure it will not open during transit.
2. Place the taped rack inside the provided plastic bag and seal tightly.
3. Freeze the rack in a freezer until shipping. Note: A −80° C. freezer is preferred but −20° C. is acceptable for short term storage of spent culture media samples up to 2 weeks.
4. Ship the rack with frozen gel packs to the testing laboratory in the provided boxes. Sample pickup can be scheduled by emailing.

Outcome Data Collection
1. Pregnancy outcome data for embryos transferred fresh or in subsequent frozen embryo transfer cycles will be collected on a web-based portal. Participating clinics may submit their pregnancy outcome data either directly into the web-based portal or submit by paper form to the BioIncept Scientific Liaison who will collate the outcome data.
2. Data to be collected include:
   a. At least 2 sequential βhCG levels.
   b. Ultrasound at 6-8 week documenting intrauterine gestational sac and fetal cardiac activity.
   c. Live birth outcome including birth date, weight and gender will be collected.

Endpoints
1. The primary endpoint is to confirm that embryos that do not produce PIF do not implant. Implantation will be initially confirmed by βHCG production. Implantation will be further confirmed by an appropriate rise in βHCG levels and visualization of a gestational sac and fetal heart rate on ultrasound at 6-8 weeks estimated gestational age.
2. The secondary endpoints will be to correlate levels of PIF at the blastocyst stage with pregnancy outcome (implantation, development of fetal cardiac activity and birth). An additional secondary endpoint is to correlate PIF production with embryo blastocyst morphology. We are especially interested in whether embryo morphology correlates with PIF production.

### REFERENCES

1. Roussev R. G., et al., “Embryonic origin of preimplantation factor (PIF): biological activity and partial characterization,” Molecular Human Reproduction, 1996, 2(11): 883-887.
2. Barnea E. R., et al., “Insight into preimplantation factor (PIF*) mechanism for embryo protection and development: target oxidative stress and protein misfolding (PDI and HSP) through essential RIKP [Corrected] binding site,” PLoS One, 2014, 1; 9(7):e100263.
3. Ramu S., et al., “Preimplantation factor (PIF) detection in maternal circulation in early pregnancy correlates with live birth (bovine model),” Reproductive Biology and Endocrinology, 2013, 11:105.
4. Stamatkin C. W., et al., “PreImplantation factor (PIF) correlates with early mammalian embryo development-bovine and murine models,” Reproductive Biology and Endocrinology, 2011, 9(1):63.
5. Barnea E. R., “Insight into early pregnancy events: The emerging role of the embryo,” Am. J. Reprod. Immunol., 2004, 51(5): 319-322.
6. Mueller M., et al., “PreImplantation factor promotes neuroprotection by targeting microRNA let-7,” PNAS, 2014, 111(38): 13882-13887.
7. Coulam, C. B., et al., “Preimplantation factor (PIF) predicts subsequent pregnancy loss,” Am. J. Reprod. Immunol., 1995, 34(2):88-92.
8. Roussev, et al., “A novel bioassay for detection of preimplantation factor (PIF),” Am. J. Reprod. Immunol., 1995, 33(1):68-73.
9. Rosario G. X., et a., “Morphological events in the primate endometrium in the presence of a preimplantation embryo, detected by the serum preimplantation factor bioassay,” Hum. Reprod., 2005, 20(1): 61-71.
10. Moindjie H., et al., “Preimplantation factor (PIF) promotes human trophoblast invasion,” Biol. Reprod., 2014, 91(5): 118.
11. Duzyj C. M., et al., “Preimplantation factor promotes first trimester trophoblast invasion,” Am. J. Obstet. Gynecol., 2010, 203(4): 402.e1-4.
12. Barnea E. R., et al., “PreImplantation Factor (PIF*) endogenously prevents preeclampsia: Promotes trophoblast invasion and reduces oxidative stress,” J. Reprod. Immunol., 2016, 114: 58-64.
13. Stamatkin C. W., et al., “Preimplantation factor negates embryo toxicity and promotes embryo development in culture,” Reprod. Biomed. Online, 2011, 23(4): 517-24.

1. A monoclonal antibody, or an antigen-binding fragment thereof, comprising a variable region comprising:
a) a complementarity-determining region (CDR) comprising at least about 70% sequence identity to one or a combination of: amino acid sequence of SEQ ID NO: 3, SEQ ID NO: 4 or SEQ ID NO: 5; or
b) a CDR comprising at least about 70% sequence identity to the amino acid sequence of SEQ ID NO: 8, SEQ ID NO: 9 or SEQ ID NO: 10.

2. The monoclonal antibody of claim 1, comprising:
a) a heavy chain variable region comprising a CDR comprising at least about 70% sequence identity to the amino acid sequence of SEQ ID NO: 3, SEQ ID NO: 4 or SEQ ID NO: 5; and
b) a light chain variable region comprising a CDR comprising at least about 70% sequence identity to the amino acid sequence of SEQ ID NO: 8, SEQ ID NO: 9 or SEQ ID NO: 10.

3. The monoclonal antibody of claim 1, comprising:
a1) a heavy chain variable region comprising a first CDR comprising the amino acid sequence of SEQ ID NO: 3 and a second CDR comprising the amino acid sequence of SEQ ID NO: 4;
a2) a heavy chain variable region comprising a first CDR comprising the amino acid sequence of SEQ ID NO: 3 and a second CDR comprising the amino acid sequence of SEQ ID NO: 5; or
a3) a heavy chain variable region comprising a first CDR comprising the amino acid sequence of SEQ ID NO: 4 and a second CDR comprising the amino acid sequence of SEQ ID NO: 5.

4. The monoclonal antibody of claim 1, comprising:
b1) a light chain variable region comprising a first CDR comprising the amino acid sequence of SEQ ID NO: 8 and a second CDR comprising the amino acid sequence of SEQ ID NO: 9;
b2) a light chain variable region comprising a first CDR comprising the amino acid sequence of SEQ ID NO: 8 and a second CDR comprising the amino acid sequence of SEQ ID NO: 10; or
b3) a light chain variable region comprising a first CDR comprising the amino acid sequence of SEQ ID NO: 9 and a second CDR comprising the amino acid sequence of SEQ ID NO: 10.

5. The monoclonal antibody of claim 1, comprising a heavy chain variable region comprising a first CDR comprising the amino acid sequence of SEQ ID NO: 3, a second CDR comprising the amino acid sequence of SEQ ID NO: 4, and a third CDR comprising the amino acid sequence of SEQ ID NO: 5.

6. The monoclonal antibody of claim 1, comprising a light chain variable region comprising a first CDR comprising the amino acid sequence of SEQ ID NO: 8, a second CDR comprising the amino acid sequence of SEQ ID NO: 9, and a third CDR comprising the amino acid sequence of SEQ ID NO: 10.

7. The monoclonal antibody of claim 1, comprising a heavy chain variable region comprising at least about 70% sequence identity to SEQ ID NO: 2.

8. The monoclonal antibody of claim 1, comprising a light chain variable region comprising at least about 70% sequence identity to SEQ ID NO: 7.

9. The monoclonal antibody of claim 1, comprising a heavy chain variable region comprising at least about 90% sequence identity to SEQ ID NO: 2, and a light chain variable region comprising at least about 90% sequence identity to SEQ ID NO: 7.

10. The monoclonal antibody of claim 1, comprising a heavy chain variable region comprising SEQ ID NO: 2, and a light chain variable region comprising SEQ ID NO: 7.

11. The monoclonal antibody of claim 1, further comprising a heavy chain constant region and a light chain constant region.

12. (canceled)

13. The monoclonal antibody of claim 1, wherein the monoclonal antibody is a rabbit monoclonal antibody, wherein the monoclonal antibody is capable of binding to a preimplantation factor (PIF), or an immunogenic fragment or epitope thereof, and wherein the monoclonal antibody is specific to the PIF comprising the amino acid sequence of SEQ ID NO: 11.

14. (canceled)

15. (canceled)

16. A system comprising:
a) one or a plurality of monoclonal antibodies of claim 1;
b) a solid support onto which the one or plurality of monoclonal antibodies are immobilized; and
c) at least one substrate.

17. The system of claim 16, further comprising a fluorimeter or spectrophotometer.

18. The system of claim 16, wherein the at least one substrate is capable of binding to the one or plurality of monoclonal antibodies; wherein the at least one substrate is fluorogenic, fluorescent, or chemiluminescent fluorescently labeled; and wherein the at least one substrate is a preimplantation factor (PIF), or an immunogenic fragment or epitope thereof.

19-76. (canceled)


SEQUENCE LISTING (55 sequences)

SEQ ID NO: 1 - DNA, 825 bp, Homo sapiens
atggagactgggctgcgctggcttctcctggtcgctgtgctcaaaggtgtccagtgtcggtcgctggaggagtccgggggtcgcctggtcacgcctgggacacccctgacactcacctgcacagtctctggaatcgacctcagcacctattcaatgagttgggtccgccaggctccaggggagggcctggaatggatcgccaccattaatactggtggtagcgcatactacgcgaactgggcgaaaggccgattcaccatctccaaaacctcgaccacggtgactctgaaaatgaccagtctgacaaccgaggacacggccacctatttctgtgccagaacgattagtgctgataatttgtggggccacggcaccctggtcaccgtctcctcagggcaacctaaggctccatcagtcttcccactggccccctgctgcggggacacacccagctccacggtcaccctgggttgtcttgtgaagggatacctcccggaacccgtgaccgtgacctggaactccggcaccctgaccaatggagtgcggaccttcccgagcgtcaggcagtcctccgggttgtacagcttgtctagcgtggtgtccgtgacgtcgtcaagccagcctgtgacttgcaatgtggcccatccggccaccaacaccaaggtcgacaagaccgtggcgccttccacctgttccaagcccacttgcccgccgcctgagctcctgggaggaccgtccgtgttcatcttccctccaaaacccaaggataccctgatgattagccgcactcccgaagtcacttgcgtggtcgtggacgtgtcgcag

SEQ ID NO: 2 - protein, 275 aa, Homo sapiens
METGLRWLLLVAVLKGVQCRSLEESGGRLVTPGTPLTLTCTVSGIDLSTYSMSWVRQAPGEGLEWIATINTGGSAYYANWAKGRFTISKTSTTVTLKMTSLTTEDTATYFCARTISADNLWGHGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQ

SEQ ID NO: 3 - protein, 8 aa, Homo sapiens
GIDLSTYS

SEQ ID NO: 4 - protein, 7 aa, Homo sapiens
INTGGSA

SEQ ID NO: 5 - protein, 9 aa, Homo sapiens
ARTISADNL

SEQ ID NO: 6 - DNA, 708 bp, Homo sapiens
atggacacgagggcccccactcagctgctggggctcctgctgctctggctcccaggtgccacatttgcccaagtgctgacccagactccatcctccgtgtctgcagctgtgggaagcacagtcaccatcagttgccagtccagtcagagtgtttatagtgactacttatcttggtatcagcagaaaccaggacagcctcccaagcgcctgatatatgctgcatttactctggcatctggtgtcccatcgcggttcaaaggcagtggatctgagaaacagttcactctcaccatcagcgacctggagtgtgacgatgctgccacttactactgtgcagccatttatagtaatgatatttgggctttcggcggagggaccgaggtggtcgtcaaaggtgatccagttgcacctactgtcctcatcttcccaccatcagcggaccaagtggccactggcaccgtgaccattgtctgcgtggctaacaagtacttcccggacgtgaccgtgacctgggaagtcgacggaaccactcagaccactggtatcgagaacagcaagacgccccagaactccgccgattgtacttataacctgtcctccacactgaccctcacctcgacccagtacaattcccacaaggagtacacttgcaaagtcacccagggaaccacttcagtggtgcagagcttcaaccggggggattgctga

SEQ ID NO: 7 - protein, 235 aa, Homo sapiens
MDTRAPTQLLGLLLLWLPGATFAQVLTQTPSSVSAAVGSTVTISCQSSQSVYSDYLSWYQQKPGQPPKRLIYAAFTLASGVPSRFKGSGSEKQFTLTISDLECDDAATYYCAAIYSNDIWAFGGGTEVVVKGDPVAPTVLIFPPSADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 8 - protein, 7 aa, Homo sapiens
QSVYSDY

SEQ ID NO: 9 - protein, 3 aa, Homo sapiens
AAF

SEQ ID NO: 10 - protein, 10 aa, Homo sapiens
AAIYSNDIWA

SEQ ID NO: 11 - protein, 15 aa, Homo sapiens
MVRIKPGSANKPSDD

SEQ ID NO: 12 - protein, 10 aa, Homo sapiens
SGIDLSTYSN

SEQ ID NO: 13 - protein, 8 aa, Homo sapiens
VSGIDLST

SEQ ID NO: 14 - protein, 8 aa, Homo sapiens
IDLSTYSM

SEQ ID NO: 15 - protein, 7 aa, Homo sapiens
STYSMSW

SEQ ID NO: 16 - protein, 12 aa, Homo sapiens
VSGIDLSTYSMS

SEQ ID NO: 17 - protein, 9 aa, Homo sapiens
IDLSTYSMS

SEQ ID NO: 18 - protein, 10 aa, Homo sapiens
GIDLSTYSMS

SEQ ID NO: 19 - protein, 10 aa, Homo sapiens
TINTGGSAYY

SEQ ID NO: 20 - protein, 9 aa, Homo sapiens
NTGGSAYYA

SEQ ID NO: 21 - protein, 7 aa, Homo sapiens
TGGSAYY

SEQ ID NO: 22 - protein, 13 aa, Homo sapiens
IATINTGGSAYYA

SEQ ID NO: 23 - protein, 6 aa, Homo sapiens
NTGGSA

SEQ ID NO: 24 - protein, 11 aa, Homo sapiens
TINTGGSAYYA

SEQ ID NO: 25 - protein, 9 aa, Homo sapiens
INTGGSAYY

SEQ ID NO: 26 - protein, 11 aa, Homo sapiens
CARTISADNLW

SEQ ID NO: 27 - protein, 10 aa, Homo sapiens
ATYFCARTIS

SEQ ID NO: 28 - protein, 9 aa, Homo sapiens
ADNLWGHGT

SEQ ID NO: 29 - protein, 8 aa, Homo sapiens
RTISADNL

SEQ ID NO: 30 - protein, 11 aa, Homo sapiens
TISADNLWGHG

SEQ ID NO: 31 - protein, 8 aa, Homo sapiens
ARTISADN

SEQ ID NO: 32 - protein, 13 aa, Homo sapiens
ARTISADNLWGHG

SEQ ID NO: 33 - protein, 9 aa, Homo sapiens
SSQSVYSDY

SEQ ID NO: 34 - protein, 11 aa, Homo sapiens
SQSVYSDYLSW

SEQ ID NO: 35 - protein, 9 aa, Homo sapiens
QSVYSDYLS

SEQ ID NO: 36 - protein, 8 aa, Homo sapiens
SVYSDYLS

SEQ ID NO: 37 - protein, 9 aa, Homo sapiens
VYSDYLSWY

SEQ ID NO: 38 - protein, 9 aa, Homo sapiens
YSDYLSWYQ

SEQ ID NO: 39 - protein, 9 aa, Homo sapiens
SDYLSWYQQ

SEQ ID NO: 40 - protein, 9 aa, Homo sapiens
LIYAAFTLA

SEQ ID NO: 41 - protein, 7 aa, Homo sapiens
KRLIYAA

SEQ ID NO: 42 - protein, 10 aa, Homo sapiens
IYAAFTLASG

SEQ ID NO: 43 - protein, 8 aa, Homo sapiens
AFTLAASG

SEQ ID NO: 44 - protein, 8 aa, Homo sapiens
LIYAAFTL

SEQ ID NO: 45 - protein, 9 aa, Homo sapiens
YAAFTLASG

SEQ ID NO: 46 - protein, 9 aa, Homo sapiens
AAFTLASGV

SEQ ID NO: 47 - protein, 5 aa, Homo sapiens
YAAFT

SEQ ID NO: 48 - protein, 7 aa, Homo sapiens
IYAAFTL

SEQ ID NO: 49 - protein, 16 aa, Homo sapiens
AATYYCAAIYSNDIWA

SEQ ID NO: 50 - protein, 16 aa, Homo sapiens
ATYYCAAIYSNDIWAF

SEQ ID NO: 51 - protein, 16 aa, Homo sapiens
TYYCAAIYSNDIWAFG

SEQ ID NO: 52 - protein, 16 aa, Homo sapiens
YYCAAIYSNDIWAFGG

SEQ ID NO: 53 - protein, 16 aa, Homo sapiens
YCAAIYSNDIWAFGGG

SEQ ID NO: 54 - protein, 16 aa, Homo sapiens
CAAIYSNDIWAFGGGT

SEQ ID NO: 55 - protein, 16 aa, Homo sapiens
AAIYSNDIWAFGGGTE